(19) United States
(12) Patent Application Publication
(10) Pub. No.: US 2025/0288380 A1
(43) Pub. Date: Sep. 18, 2025

(54) TRANSMISSION ASSEMBLY FOR DRIVING INSTRUMENT INSERTION, AND RELATED DEVICES, SYSTEMS AND METHODS

(71) Applicant: INTUITIVE SURGICAL OPERATIONS, INC., Sunnyvale, CA (US)

(72) Inventors: William A. Burbank, Sandy Hook, CT (US); Paul A. Millman, San Jose, CA (US)

(73) Assignee: INTUITIVE SURGICAL OPERATIONS, INC., Sunnyvale, CA (US)

(21) Appl. No.: 19/080,220

(22) Filed: Mar. 14, 2025

Related U.S. Application Data
(60) Provisional application No. 63/565,678, filed on Mar. 15, 2024.

(51) Int. Cl. A61B 34/00; A61B 34/35

(52) U.S. Cl. CPC A61B 34/71; A61B 34/35

(57) ABSTRACT

A medical instrument comprises an instrument shaft, a movable component coupled to the instrument shaft, a pushable actuation element coupled to the moveable component and translatable relative to the instrument shaft, a first set of pullable actuation elements coupled to the instrument shaft, a second set of pullable actuation elements coupled to the pushable actuation element, and a transmission assembly movably coupled to the instrument shaft. Translation of the pushable actuation element drives a degree of freedom of motion of the movable component. The transmission assembly comprises a first rotatable drive member configured to actuate the first set of pullable actuation elements to drive translation of the instrument shaft relative to the transmission assembly, and one or more capstans configured to actuate the second set of pullable actuation elements to drive translation of the pushable actuation element relative to the instrument shaft.

### CROSS-REFERENCE TO RELATED APPLICATIONS

This application claims priority to U.S. Provisional Application No. 63/565,678, filed Mar. 15, 2024, which is incorporated herein by reference in its entirety.

### FIELD

Aspects of this disclosure relate generally to instruments for use with computer-assisted teleoperated manipulator systems, and related devices, systems, and methods. More specifically, aspects of the disclosure relate to instrument transmission assembly architectures that provide a drive for instrument insertion.

### INTRODUCTION

Some remotely-controlled instruments comprise a shaft, an end effector coupled to the shaft, and a transmission assembly coupled to the shaft. The end effector generally comprises one or more functional elements, such as, for example, a jaw mechanism, a stapler, a cutting implement, a camera, an electrode, a sensor, etc., to perform one or more functions of the instrument, such as cutting, sealing, grasping, imaging, etc. The transmission assembly may comprise one or more drive members configured to receive driving forces, torques, or other inputs and transmit the received driving forces, torques, or other inputs to other portions of the instrument, such as the end effector and/or other structures along portions of the shaft (e.g., joints and or wrist mechanisms), to drive degrees of freedom of motion and/or functions of the instrument. The drive members may transmit received driving forces or torques to other portions of the instrument via actuation elements running from the transmission assembly along the instrument shaft. Such actuation elements may be in the form of pulling (tension) members such as cables, wires, filaments or the like that are flexible in all directions and generally transmit force by pulling on the actuation element to place it in tension (sometimes referred to as pull-pull actuation elements), more rigid members such as rods, sheet metal strips, or the like that can transmit force by pushing or by pulling on the actuation element (sometimes referred to as push-pull actuation elements), flexible push members such as push coils that can transmit force by pushing while flexing laterally, rotatable members such as lead screws that can transmit rotational force, and so on. In some cases, the drive members may convert received driving forces or torques from one form to another, such as, for example, converting received torques into a linear forces.

The transmission assembly of such instruments can be configured to be coupled to computer-assisted teleoperated manipulator systems to receive the drive inputs, such as via servo-motor and drive disk interfaces, and/or may be configured to receive input manually, such as via a user operating a wheel, button, trigger, or other mechanism at the force transmission assembly to provide the desired input.

Some remotely-controlled instruments have a degree of freedom of motion comprising linear translation of the instrument shaft relative to a workspace (e.g., a patient's body) along a longitudinal axis of the shaft. Such translation of the instrument shaft, and hence the end effector coupled thereto, may allow for insertion of the end effector into (or advancement further into) the workspace and withdrawal of the end effector from the workspace. This degree of freedom of motion may be referred to herein as an insertion degree of freedom, and the direction along which the translation occurs may be referred to herein as an insertion axis.

Some instruments, and manipulator systems, provide for the insertion degree of freedom of motion by translating the entire instrument, including the transmission assembly, along the insertion axis. For example, for a teleoperated manipulator system, an instrument holder to which the instrument is coupled, may be translatable along a link of a manipulator of the manipulator system. Likewise, for a manually-operated instrument, insertion may occur via movement of the entire instrument including the transmission assembly, via a user generally holding the instrument at least in part by the transmission assembly housing.

However, other instruments and manipulator systems provide for the insertion degree of freedom of the instrument shaft and end effector via drive input from the transmission assembly itself, thereby causing relative translation between the transmission assembly of the instrument and the shaft of the instrument. In such systems, the relative translation between the instrument shaft and the transmission assembly causes translation of the instrument shaft relative to the workspace because the transmission assembly is held translationally stationary relative to the workspace by the manipulator to which the instrument is coupled.

The ability of the transmission assembly and instrument shaft to translate relative to each other results in a relatively complex architecture of drive and force transmission structures that make up the transmission assembly. There exists a need to provide instruments with transmission assemblies that drive the insertion degree of freedom movement of the instrument while still being relatively simple and compact in overall architecture to provide the drive forces associated with the other functionality of the instrument, and/or to otherwise improve performance of instruments with such transmission assembly architectures.

### SUMMARY

Various embodiments of the present disclosure may solve one or more of the above-mentioned problems and/or may demonstrate one or more of the above-mentioned desirable features. Other features and/or advantages may become apparent from the description that follows.

In accordance with at least one embodiment of the present disclosure a medical instrument comprises an instrument shaft, a movable component coupled to the instrument shaft, a pushable actuation element coupled to the moveable component and translatable relative to the instrument shaft, a first set of pullable actuation elements coupled to the instrument shaft, a second set of pullable actuation elements coupled to the pushable actuation element, and a transmission assembly movably coupled to the instrument shaft. Translation of the pushable actuation element drives a degree of freedom of motion of the movable component. The transmission assembly comprises a first rotatable drive member configured to actuate the first set of pullable actuation elements to drive translation of the instrument shaft relative to the transmission assembly, and one or more capstans configured to actuate the second set of pullable actuation elements to drive translation of the pushable actuation element relative to the instrument shaft.

In accordance with at least one embodiment of the present disclosure, a medical instrument comprises an instrument shaft, a movable component coupled to the instrument shaft, a pushable actuation element coupled to the moveable component and translatable relative to the instrument shaft, a first set of pullable actuation elements coupled to the instrument shaft, a second set of pullable actuation elements comprising a first pullable actuation element and a second pullable actuation element, and a transmission assembly movably coupled to the instrument shaft. The pushable actuation element is configured to drive a degree of freedom of motion of the movable component in response to translation of the pushable actuation element relative to the instrument shaft. The transmission assembly comprises a first drive member configured to actuate the first set of a pullable actuation elements to drive translation of the instrument shaft relative to the transmission assembly, a second drive member comprising a first capstan coupled to the first pullable actuation element, and a third drive member comprising a second capstan coupled to the second pullable actuation element. The first and second pullable actuation elements are coupled to the pushable actuation element such that simultaneous actuation of the first and second pullable actuation elements in opposite directions by the first and second capstans drives translation of the pushable actuation element relative to the instrument shaft.

In accordance with at least one embodiment of the present disclosure a medical instrument comprises an instrument shaft, a movable component coupled to the instrument shaft, a pushable actuation element coupled to the moveable component and translatable relative to the instrument shaft, a first distal pulley coupled to a distal portion of the pushable actuation element, a first proximal pulley coupled to a proximal portion of the pushable actuation element, a second distal pulley coupled to a distal portion of the instrument shaft, a second proximal pulley coupled to a proximal portion of the instrument shaft, a first pullable actuation element routed between the first distal pulley and the second proximal pulley, a second pullable actuation element routed between the second distal pulley and the first proximal pulley, and a transmission assembly movably coupled to the instrument shaft. The pushable actuation element is configured to drive a degree of freedom of motion of the movable component in response to translation of the pushable actuation element relative to the instrument shaft. The transmission assembly comprises a first drive member configured to drive translation of the instrument shaft relative to the transmission assembly, and a second drive member comprising one or more capstans and a drive input configured to drive rotation of the one or more capstans to actuate the first pullable actuation element and the second pullable actuation element to drive translation of the pushable actuation element.

In accordance with at least one embodiment of the present disclosure a method of operating a medical instrument is provided. The medical instrument comprises an instrument shaft, a force transmission assembly coupled to a proximal end portion of the instrument shaft and an end effector coupled to a distal end portion of the instrument shaft. The method comprises translating the instrument shaft relative of the force transmission assembly, and translating a pushable actuation element relative to the instrument shaft to drive a degree of freedom of motion of a movable component of the end effector. While translating the instrument shaft relative to the force transmission assembly, the pushable actuation element remains stationary relative to the instrument shaft.

### BRIEF DESCRIPTION OF THE DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more embodiments of the present teachings and together with the description explain certain principles and operation. In the drawings:

FIG. 1A is a schematic diagram of an embodiment of an instrument;

FIG. 1B is a schematic diagram of a drive member of the instrument of FIG. 1A;

FIG. 1C is a schematic diagram of another drive member of the instrument of FIG. 1A;

FIG. 1D is a schematic diagram of yet another drive member of the instrument of FIG. 1A;

FIG. 1E is a schematic diagram of portions of the instrument of FIG. 1A;

FIG. 2 is a perspective view of another embodiment of an instrument;

FIG. 3 is a perspective view of a transmission assembly housing and a portion of the shaft of the instrument of FIG. 2;

FIG. 4 is a perspective view of an embodiment of a transmission assembly of the instrument of FIG. 2 with a cover portion removed;

FIG. 5 is a plan, partial cross-sectional view of the transmission assembly of FIG. 4 with a cover portion removed and the section taken along the line 5-5;

FIG. 6 is a schematic diagram of portions of the instrument of FIG. 2;

FIG. 7A is a plan view of an embodiment of an actuation transfer mechanism of the instrument of FIG. 2 in a first state;

FIG. 7B is a plan view of the actuation transfer mechanism of FIG. 7A in a second state;

FIG. 7C is a side view of the actuation transfer mechanism of FIG. 7A in the first state;

FIG. 7D is a side view of the actuation transfer mechanism of FIG. 7A in the second state;

FIG. 8A is a schematic diagram of portions of the instrument of FIG. 2 illustrating how a pushable actuation element is actuated in a distal direction;

FIG. 8B is a schematic diagram of portions of the instrument of FIG. 2 illustrating how a pushable actuation element is actuated in a proximal direction;

FIG. 9A is an enlarged perspective view of a portion of the instrument of FIG. 2 with a shaft housing and end cap removed;

FIG. 9B is an enlarged perspective view of the portion of the instrument of FIG. 9A with a shaft housing and end cap made transparent and with a pushable actuation element in a most proximal position;

FIG. 9C is an enlarged perspective view of the portion of the instrument of FIG. 9B with the pushable actuation element in a most distal position and a transmission assembly in a most proximal position with a housing of the transmission assembly omitted;

FIG. 9D is an enlarged perspective view of another portion of the instrument of FIG. 2 with the pushable actuation element in a most proximal position;

FIG. 9E is an enlarged perspective view of the portion of the instrument of FIG. 9D with the pushable actuation element in a most distal position;

FIG. 9F is an enlarged perspective view of the portion of the instrument of FIG. 9E with the pushable actuation element omitted and with the transmission assembly in a most distal position with a housing of the transmission assembly omitted;

FIG. 10A is an enlarged side view of the portion of the instrument of FIG. 9D from a first perspective with the pushable actuation element in a most proximal position;

FIG. 10B is an enlarged side view of the portion of the instrument of FIG. 10A from the first perspective with the pushable actuation element in a most distal position;

FIG. 10C is an enlarged side view of the portion of the instrument of FIG. 9D from a second perspective, rotated 90 degrees relative to the first perspective, with the pushable actuation element in the most proximal position;

FIG. 10D is an enlarged side view of the portion of the instrument of FIG. 10C from the second perspective with the pushable actuation element in the most distal position;

FIG. 11 is a perspective view of an embodiment of an end effector;

FIG. 12A is a cross-section of the end effector of FIG. 11 in an open state, with the section taken along the line 12-12 in FIG. 11;

FIG. 12B is a cross-section of the end effector of FIG. 11 in a closed state, with the section taken along the line 12-12 in FIG. 11;

FIG. 13 is a section view of an embodiment of a transmission assembly, with the section taken along a longitudinal centerline of an instrument shaft;

FIG. 14 is a plan view of an embodiment of a transmission assembly, with instrument shaft omitted;

FIG. 15 is a section of an embodiment of a transmission assembly, with the section taken along the line 15-15 in FIG. 14;

FIG. 16A is a schematic diagram of portions of the transmission assembly of FIG. 14 illustrating how a pushable actuation element is actuated in a distal direction;

FIG. 16B is a schematic diagram of portions of the transmission assembly of FIG. 14 illustrating how a pushable actuation element is actuated in a proximal direction;

FIG. 17 is a plan view of an embodiment of a transmission assembly, with instrument shaft omitted;

FIG. 18 is a section view of an embodiment of a transmission assembly, with the section taken along the line 18-18 in FIG. 17;

FIG. 19A is a schematic diagram of portions of the transmission assembly of FIG. 17 illustrating how a pushable actuation element is actuated in a distal direction;

FIG. 19B is a schematic diagram of portions of the transmission assembly of FIG. 17 illustrating how a pushable actuation element is actuated in a proximal direction;

FIG. 20 is a section view of an embodiment of a transmission assembly, with the section taken along a longitudinal centerline of an instrument shaft;

FIG. 21A is a schematic diagram of portions of the transmission assembly of FIG. 20 illustrating how a pushable actuation element is actuated in a distal direction;

FIG. 21B is a schematic diagram of portions of the transmission assembly of FIG. 20 illustrating how a pushable actuation element is actuated in a proximal direction;

FIG. 22A is a schematic diagram of portions of another embodiment of a transmission assembly illustrating how a pushable actuation element is actuated in a distal direction;

FIG. 22B is a schematic diagram of portions of the transmission assembly of FIG. 22A illustrating how a pushable actuation element is actuated in a proximal direction;

FIG. 23A is schematic diagram illustrating a portion of another embodiment of a transmission assembly;

FIG. 23B is schematic diagram illustrating a portion of yet another embodiment of a transmission assembly;

FIG. 24 is a schematic block diagram of an embodiment of a teleoperable manipulator system; and

FIG. 25 is a partial, perspective view of an embodiment of an instrument manipulator.

### DETAILED DESCRIPTION

In the description below, reference is made to “paying out” or “drawing in” pullable actuation elements, such as cables, wires, filaments, belts, etc. Paying out an actuation element refers to increasing the length of a segment of the actuation element that extends between a take-off point on a drive member to which the actuation element is coupled and a coupling point at which the actuation element is operably coupled to a moveable component whose motions are driven by the actuation element. Conversely, drawing in the actuation element refers to decreasing the length of the segment of the actuation element that extends between the take-off point of the drive member and the coupling point of the driven moveable component. By extension, “paid-out length” refers to the current length of the above-described segment of the actuation element that extends between the take-off point and the coupling point. Furthermore, in the description below, reference is made to the “path length” for an actuation element. The path length refers to the length of a hypothetical path that the above-described segment would be expected to traverse as it extends between the take-off point and the coupling point, assuming that the segment has no slack. The path length does not necessarily always equal the paid-out length, as some slack may be present resulting in the paid-out length exceeding the path length. References to “actuating” an actuation element refer to causing the actuation element to be paid out or drawn in. References to “actuating” a drive member refer to causing the drive member to move (e.g., rotate) so as to perform some action, such as actuating an actuation element coupled thereto. The above-described terms and definitions are explained in further detail below, in and following the description of the various embodiments.

As noted above, in instruments for which an insertion degree of freedom occurs by a drive input at a transmission assembly of the instrument causing relative translation between the transmission assembly and the instrument shaft, the transmission assembly can result in a complex architecture. Some aspects of one embodiment of such a transmission assembly are described briefly below to illustrate some of the challenges that may arise in such instruments. In an embodiment of a transmission assembly, one of the drive members, also referred to hereinafter as the insertion drive member, drives translation of the instrument shaft relative to the transmission assembly to achieve the insertion degree of freedom motion of the instrument shaft and end effector. Various other drive members of such a transmission assembly, also referred to hereinafter as non-insertion drive members, can each be coupled to at least one respectively corresponding actuation element in the form of pull-pull actuation elements that are actuated (drawn in or paid out) to drive a degree of freedom of motion (or other function) of the instrument. The insertion drive member may comprise a drum to which multiple pull-pull actuation elements are coupled. The actuation elements coupled to the drum include one actuation element coupled to a proximal portion of the instrument shaft to drive relative translation of the shaft and transmission assembly in one direction, and also at least some of the actuation elements that are coupled to the non-insertion drive members (in some cases, all of the actuation elements that are coupled to the non-insertion drive members). The insertion drive member and the non-insertion drive members are configured such that rotation of the insertion drive member actuates (pays out or draws in) all of the actuation elements coupled thereto independently of rotation of the non-insertion drive members, thereby causing relative translation between the shaft and transmission assembly but without consequent actuation of the movable components (e.g., end effector, wrist, etc.) coupled to the non-insertion drive members. Individual actuation elements coupled to the non-insertion drive members can also be actuated independently of the insertion drive member to cause actuation of a movable component by rotation of a corresponding one of the non-insertion drive members. In such a transmission assembly, there can be numerous actuation element segments extending between the insertion drive member and the non-insertion drive members as well as segments extending between the various drive members and the instrument shaft. It can be challenging to arrange the drive members and the actuation elements of the transmission assembly in an orderly manner that allows the cables and drive members to function as intended without tangling or otherwise interfering with one another. This can be particularly challenging when trying to design a more compact transmission assembly or when trying to include a large number of drive members or other components in the transmission assembly, as generally speaking the smaller the transmission assembly is, or the more drive members or other components are included therein, the more likely it is that an interference may occur with the actuation elements routed through the transmission assembly.

Another challenge for instruments in which the transmission assembly drives the insertion degree of freedom pertains to how to arrange and drive pushable actuation elements, such as rods, tubes, push-coils, and the like. Because the transmission assembly moves relative to the shaft, conventional means of coupling drive inputs directly to these more pushable actuation elements may not be usable, as such coupling may result in the pushable actuation elements moving with the transmission assembly relative to the shaft when the transmission assembly is moved along the insertion degree of freedom of motion, causing inadvertent actuation of the movable components coupled to these pushable actuation element. Thus, in some conventional instruments in which the transmission assembly drives the insertion degree of freedom, pushable actuation elements may not be usable, limiting the options for functionalities of the instrument.

Moreover, some types of end effectors, such as some staplers for example, may be configured to be driven by relatively large driving forces supplied by a pushable actuation element, such as driving forces on the order or 50 pounds, 100 pounds, or even greater driving forces. Moreover, in some types of end effectors, such as some staplers for example, a pushable actuation element driving a degree of freedom of the end effector may need to translate a relatively long distance, such as distances on the order of an inch, two inches, or even longer distances. However, it may be difficult to provide a relatively compact transmission assembly that can translate relative to the instrument shaft while also being able to adequately supply such large driving forces and/or such long driving distances to the pushable actuation element. When both high driving forces and long driving distances are needed, the challenge may be exacerbated. For example, some drive members that may be capable of providing high driving force to a pushable actuation element may not be able to provide a long driving distance, and conversely some drive members that may be capable of providing a long driving distance may not be capable of providing a high driving force. Moreover, drive members that may be capable of providing high driving forces and/or high driving distances may not be compatible with a transmission assembly that translates relative to a driving shaft.

Another challenge pertains to slack that can arise in actuation elements when tension is not actively applied to the actuation element, such as, for example, may occur when the instrument is not mounted to a manipulator in the case of a teleoperated instrument. If sufficient slack is developed in an actuation element, the actuation element can derail from a handling component, such as a pulley or the drive members. Slack can occur as a result of movable components controlled by the actuation elements being moved while the drive members are free to rotate, such as in the unmounted state of the instrument relative to the manipulator. By way of example, a moveable component of an instrument that is coupled to actuation elements not being actively held in tension (such as due to being mounted to a manipulator and/or otherwise having drive members clocked to a degree of rotation that tightens the actuation elements) may experience an external force, for example because the instrument has accidentally been bumped against something. Because the drive members may be free to rotate (e.g., when in an unmounted state from a manipulator system or otherwise in an unlocked state), the force applied to the moveable component may cause it to move. Such movement in turn causes one of the actuation elements coupled thereto to pay out, while generating slack in the other actuation element coupled thereto. More specifically, as the moveable component moves, the path length for a first actuation element increases and the moveable component pulls against the first actuation element. Because the corresponding drive member is free to rotate, the first actuation element is allowed to pay out in response to the pulling. However, the same motion of the moveable component decreases the path length for a second actuation element of the pair coupled to operate the moveable component. The paid-out length of the second actuation element does not change and, therefore as the path length of the second actuation element decreases, a difference between the paid-out length of the second actuation element and the path length for the first actuation element increases, resulting in slack in the second actuation element. If enough slack develops, the second actuation element may be susceptible to derailing from a handling device.

To address some of the issues noted above and otherwise improve remotely controlled instruments, some embodiments disclosed herein comprise at least one drive member that is configured to drive multiple pullable actuation elements simultaneously. For example, in some embodiments disclosed herein at least one drive member has multiple actuation transfer mechanisms driven by the same drive input of the drive member, with each actuation transfer mechanism coupled to a respective actuation element. Thus, when the drive input of the drive member is actuated, this results in the simultaneous actuation all of the actuation elements coupled to the actuation transfer mechanisms of the drive member. For example, in some embodiments, a pair of actuation elements that drives the same degree of freedom of motion of a movable component along opposite directions may be coupled to the same drive member, with the drive member being configured to actuate the actuation elements in opposite directions (i.e., actuation of the drive member in one direction causes one of the actuation elements of the pair to pay out while simultaneously causing the other actuation element of the pair to be drawn in). Thus, in such embodiments, the actuation of a single drive member may control motion of a movable component along a degree of freedom of motion, rather than having to actuate two drive members in concert with one another to control such motion. Thus, fewer drive members are needed and the transmission assembly can be made smaller, or additional parts and functionality may be added within the same size transmission assembly (e.g., the same number of drive members may be used as in prior designs, but they may drive more degrees of freedom of motion than otherwise would be possible).

In addition, in embodiments in which a pair of actuation elements that drives the same degree of freedom of motion of a movable component are coupled to and driven by the same drive member, the issue described above related to slack developing in actuation elements may be alleviated. Because the drive member is configured such that motion thereof in one direction drives actuation of the pair of actuation elements in opposite directions as one another, if one of the actuation elements ends up being inadvertently pulled to pay out a certain amount (e.g., due to the movable component being accidentally moved in an unconstrained state), this motion will result in the drive member moving in a manner that causes the other paired actuation element to be drawn in by the same amount, and vice versa, thus automatically preventing (taking up) any slack that otherwise would have developed.

Moreover, to address some of the issues noted above and otherwise improve remotely controlled instruments, in some embodiments disclosed herein a pushable actuation element (e.g., push-push or push-pull actuation element) is coupled to the transmission assembly by flexible pullable actuation elements (e.g., cables, wires, etc.). These flexible actuation elements are routed between the pushable actuation element and one or more drive members via a system of pulleys coupled to the pushable actuation element and the instrument shaft such that actuation of the flexible actuation elements drives translation of the pushable actuation element relative to the instrument shaft. Moreover, the flexible actuation elements and system of pulleys are configured such that the pushable actuation element floats relative to the force transmission assembly, meaning that the relative translation of the force transmission assembly and shaft does not affect the position of the pushable actuation element relative to the instrument shaft. In other words, the pushable actuation element remains stationary relative to the instrument shaft while the force transmission assembly is being driven to translate relative to the instrument shaft.

More specifically, in some embodiments, an instrument comprises a first pullable actuation element, a second pullable actuation element, a pushable actuation element, a first pair of pulleys coupled to opposite end portions of the pushable actuation element, a second pair of pulleys coupled to opposite end portions of the instrument shaft, and one or more capstans coupled to the first and second pullable actuation elements. In such embodiments, the first and second pullable actuation elements are routed between the one or more capstans and the first and second pairs of pulleys such that rotation of the one or more capstans to draw in the first pullable actuation element and pay out the second pullable actuation element causes the pushable actuation element to translate in a distal direction, and rotation of the one or more capstans to pay out the first pullable actuation element and draw in the second pullable actuation element causes the pushable actuation element to translate in a proximal direction. For example, in some embodiments, the first pullable actuation element is coupled to a proximal pulley of the first pair (coupled to the pushable actuation element at a position proximal of the transmission assembly) and a distal pulley of the second pair (coupled to the instrument shaft at a position distal of the transmission assembly), and therefore drawing in the first pullable actuation element causes the pushable actuation element to translate distally relative to the instrument shaft. Similarly, in some embodiments, the second pullable actuation element is coupled to a distal pulley of the first pair (coupled to the pushable actuation element at a position distal of the transmission assembly) and a proximal pulley of the second pair (coupled to the instrument shaft at a position proximal of the transmission assembly), and therefore drawing in the second pullable actuation element causes the pushable actuation element to translate proximally relative to the instrument shaft. Moreover, in such embodiments, when the instrument shaft and transmission assembly are driven to translate relative to one another, the second pair of pulleys move along with the instrument shaft because the second pair of pulleys are fixedly coupled to the instrument shaft, and because the paid out length of the first and second pullable actuation elements changes (the capstans are stationary while the instrument shaft is driven to translate), the motion of the second pair of pulleys as they follow along with the instrument shaft causes one of the first or second pullable actuation elements to pull one of the pulleys of first pair, and hence the pushable actuation element to which the pulley is coupled, to also move along with the instrument shaft. Thus, the pushable actuation element floats relative to the transmission assembly and remains stationary relative to the instrument shaft while the insertion degree of freedom of motion is actuated. Accordingly, the above-described configuration allows for pushable actuation elements to be usable even in instruments with relative translation between the transmission assembly and the instrument shaft, thereby expanding the functionalities of the instrument. For example, in some embodiments herein, an instrument may comprise an end effector configured as a stapler, which has a stapling function that is driven by a pushable actuation element.

In some embodiments, the one or more capstans comprise a single capstan that actuates both the first and second pullable actuation elements simultaneously. In other embodiments, the one or more capstans comprise two capstans, one to actuate the first pullable actuation element and another to actuate the second pullable actuation element.

In addition, to address some of the issues noted above and otherwise improve remotely controlled instruments, in some embodiments disclosed herein, the one or more capstans comprise stepped capstans which have two or more actuation element winding portions that have different radii than one another. Such actuation element winding portions that have different radii than one another are also referred to herein as steps. In such embodiments, a given pullable actuation element may have two end portions thereof coupled to different steps of a stepped capstan. The two end portions of the given pullable actuation element are wrapped around the respective steps in opposite directions such that rotation of the capstan in one direction pays out one end portion of the cable while simultaneously drawing in the other end portion of the cable, and vice versa. Because the steps to which the two ends are coupled have different radii, the steps will pay out and draw in the two end portions of the pullable actuation element at different rates for a given rate of rotation of the capstan. Thus, rotation of the capstan in a given direction results in one end portion of the pullable actuation element being drawn in or pulled out faster than the other end portion is paid out or drawn in, and therefore the overall paid out length of the pullable actuation element changes, i.e., decreasing the paid out length if one end is pulled in faster than the other is paid out and increasing the paid out length if one end is paid out faster than the other is drawn in. The rate of change of the overall paid out length of the pullable actuation element is lower than the rate at which a given end portion is paid out or drawn in, and this difference produces a mechanical advantage that multiples the amount of force that the pullable actuation element can supply to the pushable actuation element for a given torque supplied to the capstan. Accordingly, these embodiments allow for relatively large driving forces to be supplied to pushable actuation elements even in instruments with relative translation between the transmission assembly and the instrument shaft. For example, in some embodiments herein, an instrument may comprise an end effector configured as a stapler, which has a stapling function that requires relatively large pushing forces for actuation. In some embodiments, the stepped capstans may be capable of driving the pushable actuation element such that it can transfer at least 50 pounds of pushing force and in some cases 100 pounds or more of pushing force while also being able to translate at least 1 inch and in some cases 2 or more inches.

Moreover, in some embodiments the capstans may be able to wind a substantial length of pullable actuation elements, thus allowing the pushable actuation element to which the pullable actuation elements are coupled to translate over a relatively long range of motion even while supplying relatively large pushing forces. For example, in some embodiments the capstans are configured to drive the pushable actuation element to translate over a range of motion on the order of 1 inch, 2 inches, or even longer distances.

Furthermore, to address some of the issues noted above and otherwise improve remotely controlled instruments, in some embodiments the one or more capstans comprise geared capstans. A geared capstan comprises a capstan with a drive shaft that is coupled to a drive input via a gear mechanism, instead of the drive shaft being coupled directly to the drive input. The gear mechanism may provide a mechanical advantage such that the torque generated by the drive shaft of the capstan is multiplied relative to an input drive torque supplied to the drive input. For example, in some embodiments the gear mechanism comprises a first gear with a relatively small radius coupled to the drive input and a second gear with a relatively large radius coupled to the drive shaft of the capstan, with first and second gears being meshed together. In such an embodiment, the difference in radii between the first and second gears produces the aforementioned mechanical advantage. More complicated gear mechanisms with additional gears or other components may also be used to produce a similar affect, as would be understood by one of ordinary skill in the art. Thus, like the stepped capstan described above, the gear mechanisms allow for relatively large driving forces to be supplied to pushable actuation elements even in instruments with relative translation between the transmission assembly and the instrument shaft.

Turning now to the figures, various embodiments will be described in greater detail. In the figures and in the description below, reference numerals are used to identify various parts, and in some cases in which multiple instances of the same or similar part are present, characters may be appended to the end of a base reference numeral to help to distinguish one particular instance of a part from another instance of the same or similar part. The appended characters may be omitted when the parts are being referred to generally without need to distinguish different instances of the same part. In particular, to identify different instances of the same or similar part the appended characters may take the form _N where N is any integer. For example, two drive members 400 may be referred to with the reference numbers 400_1 and 400_2 when desired to identify and differentiate specific drive members 400, or may be referred to generically by the reference number 400 when differentiation of specific instances is not desired. Moreover, the appended characters may also take the form of ′ (i.e., the apostrophe or prime character), to even further differentiate similar parts. For example, two actuation transfer mechanisms 430 of the same drive member 400_2 may be referred to with the reference numbers 430_2 and 430_2′, with the _2 indicating that the two transfer mechanism 430 are associated with the drive member 400_2 and the ′ differentiating these two transfer mechanisms 430 from one another. In addition, letters such as a, b, c, etc. may also be appended to a reference number to indicate different portions or aspects of a part. For example, distal and proximal portions of an instrument shaft 100 may be labeled as 100a and 100b, respectively.

### First Instrument Embodiment

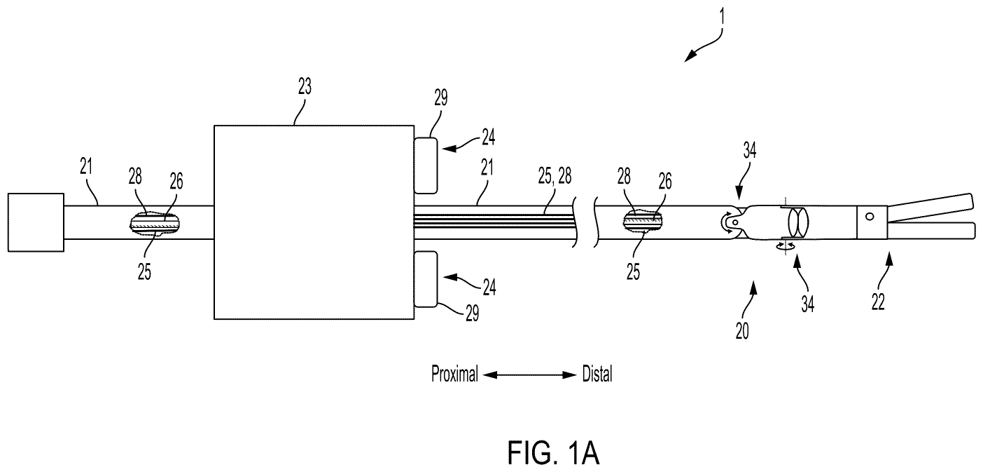

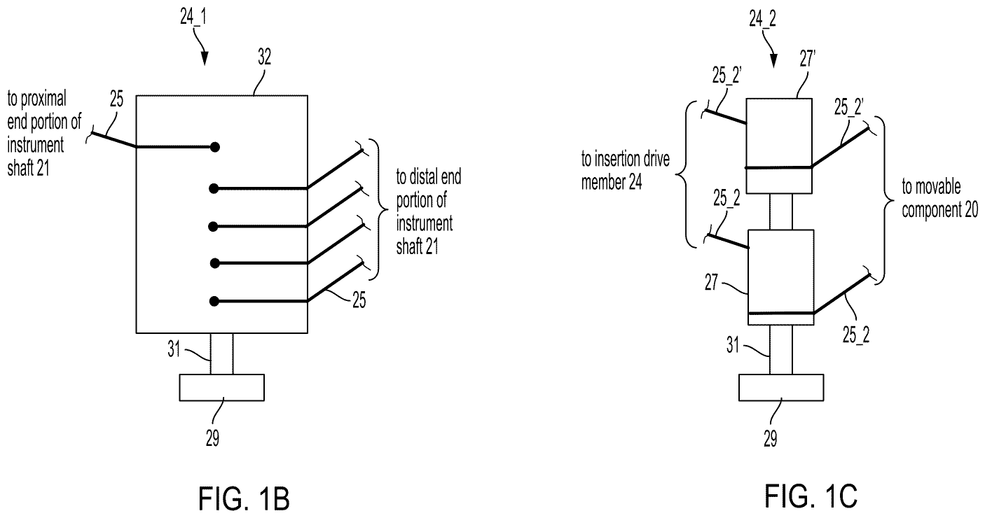

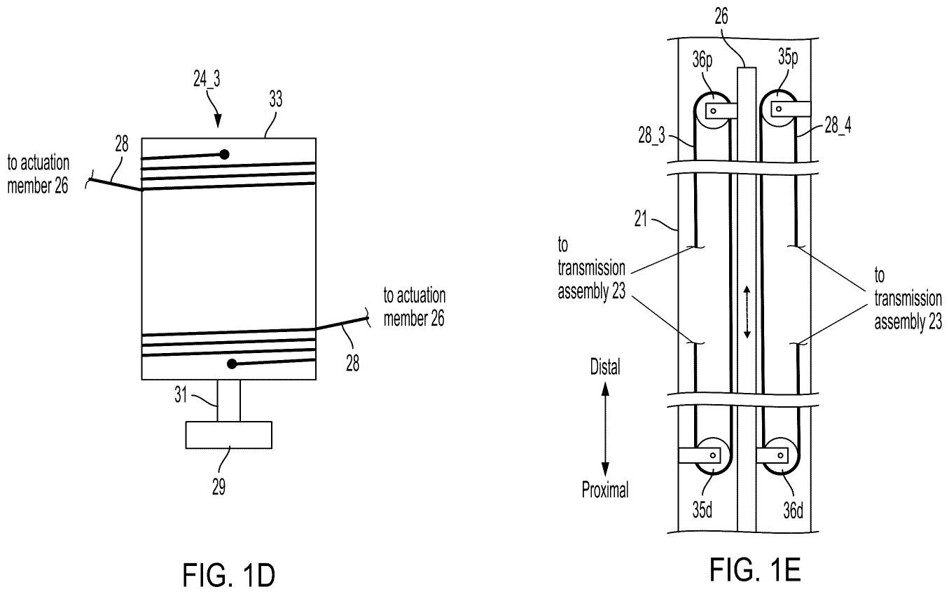

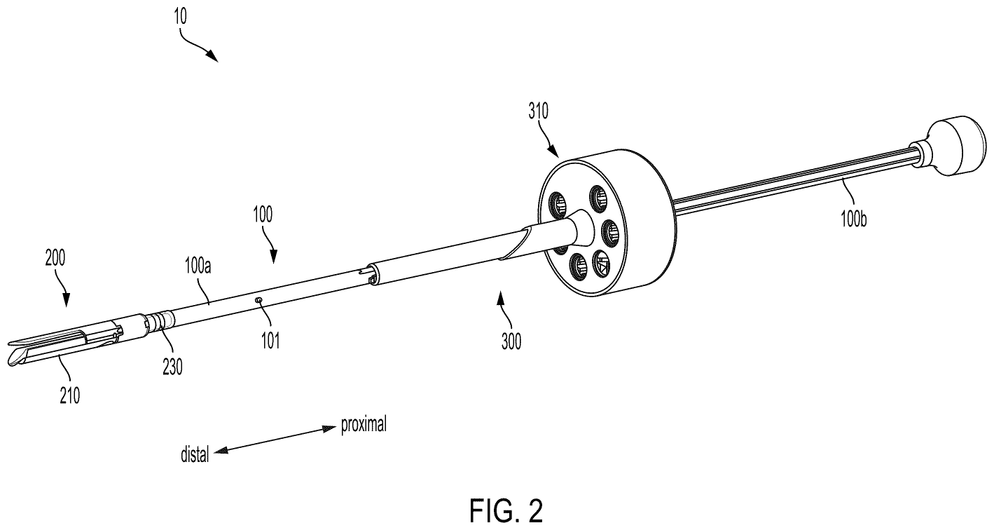

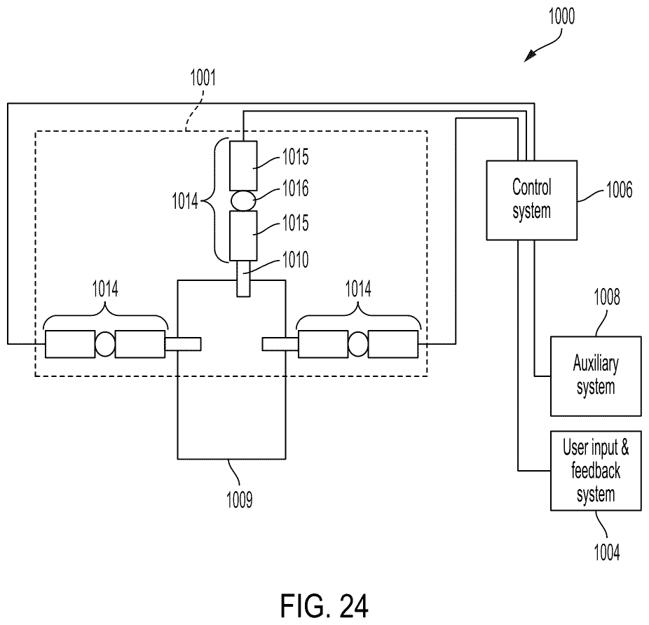

FIGS. 1A-1E illustrate an embodiment of an instrument 1. FIGS. 2-12B illustrate various views of another embodiment of an instrument 10. The instrument 10 may be used as the instrument 1. In some embodiments, it is contemplated that either of the instruments 1 or 10 is used with and controlled via a computer-controlled teleoperated system, such as the system 1000 described below in relation to FIG. 24. For example, the instruments 1 or 10 may be used as the instrument 1010 of FIG. 24. While various embodiments described herein contemplate an instrument configured to be mounted to a manipulator system of a computer-controlled teleoperated system, those having ordinary skill in the art would appreciate that a manually-operated instrument is also contemplated, in which case various drive members can be configured to be operated via manual drive inputs, such as wheels, buttons, and the like.

In some embodiments, the instrument 1 or 10 is a medical instrument, which may be used to perform medical procedures, such as, for example, surgical, diagnostic, or therapeutic procedures. Medical instruments may include a variety of instruments used to perform medical procedures, such as therapeutic instruments, diagnostic instruments, surgical instruments, and/or imaging instruments. In some examples, the medical instruments may be inserted into a patient through a natural orifice or an incision (including through a port or other guide inserted in the incision). Such instruments that are remotely controlled may be particularly useful, for example, in performing minimally invasive surgical procedures. A minimally invasive medical procedure may be designed to reduce the amount of trauma to tissue during the procedure, for example by decreasing the number and/or size of incisions through which medical instruments are inserted. In other embodiments, the instrument 1 or 10 may be a non-medical instrument, such as an industrial instrument used for remote inspection or other remote procedures.

As shown in FIG. 1A, an embodiment of an instrument 1 comprises a shaft 21 supporting an end effector 22 and one or more movable components 20 at a distal end portion of the shaft 21 (proximal and distal directions referenced herein are illustrated in FIG. 1A). The end effector 22 is configured to perform one or more functions. The one or more movable components 20 may comprise one or more components of the end effector 22 that are drivable to move (e.g., a jaw member, a translating staple firing component, etc.) and/or one or more articulable structure 34 that are drivable to articulate, such as relative to a portion of the shaft 21. The instrument 1 also comprises a force transmission assembly 23, which is movably coupled to the shaft 21 proximal of the movable components 20 (the shaft 21 and force transmission assembly 23 move relative to one another, as described below, and thus the coupling location of the transmission assembly 23 to the shaft 21 varies, but the transmission assembly 23 remains positioned generally proximal of the movable component(s) 20 of the instrument 1, and in the case of a medical instrument, the transmission assembly 23 remains outside of the body in which the distal end portion of the instrument 1 is inserted). In various embodiments, the force transmission assembly 23 is mountable to a manipulator of a computer-controlled, teleoperated system (such as the system 1000 described in further detail below), and input driving forces are provided to the instrument 1 through the manipulator interface (as also described in further detail below). In other embodiments, the interface of the force transmission assembly 23 may be configured to receive manual input to provide the input driving forces.

The end effector 22 is illustrated as having a jaw mechanism in FIG. 1A, but it should be understood that any other type of end effector could be used. Some non-limiting examples of end effectors 22 include staplers, forceps, vessel sealers, imaging devices (e.g., an endoscope tip), sensing devices (pressure, temperature, etc.), scissors, electrosurgical devices (e.g., monopolar or bipolar electrosurgical devices), other flux delivery devices (irrigation, suction, etc.), and so on. Functions performed by end effector 22 may include, for example, grasping, cutting, stapling, electrosurgical functions, illuminating, image capture, fluid delivery and/or evacuation, etc., as would be familiar to those of ordinary skill in the art. In some embodiments, one or more functions of the end effector 22 may involve motion of a movable component that is mechanically driven. In some embodiments, some functions of the end effector 22 may be driven non-mechanically, for example, by supplying electrical power or other functional flows (e.g., vacuum suction, light, etc.) to the end effector 22. Some functions of the end effector 22 may involve a combination of mechanical and non-mechanical driving inputs, such as an electrosurgical function which may comprise a mechanically driven operation of closing of a jaw member to grasp an object and an electrically driven operation of sealing or cutting the grasped object.

As shown in FIG. 1A, the force transmission assembly 23 comprises drive members 24 configured to receive input driving forces via drive inputs 29, with the driving forces controlling degrees of freedom of motion of the instrument 1. The drive members 24 convert and transfer the driving forces to actuation elements 25, 26, and 28 to drive motion of the movable components 20 or other parts of the instrument 1. Specifically, the instrument 1 comprises a first set of actuation elements 25 actuatable to drive an insertion degree of freedom of motion (as well as additional degrees of freedom of motion in some embodiments), a pushable actuation element 26 that is translatable relative to the instrument shaft 21 to drive a degree of freedom of motion of a movable component 20, and a second set of actuation elements 28 actuatable to drive translation of the pushable actuation element 26. The actuation elements 25 and 28 may be flexible pullable actuation elements such as cables, wires, filaments, etc., whereas the pushable actuation element may comprise an element capable of transferring pushing forces (e.g., beam, rod, tube, push-coil, etc.). The actuation elements 25 or 28 may also be referred to herein as “pullable actuation elements,” or “flexible pullable actuation elements”. The pushable actuation elements 26 may also be referred to herein as “actuation elements 26”. The pullable actuation elements 25 and 28 may be coupled to and driven by the drive members 24. The pushable actuation elements 26 may be coupled to and driven by a pair of the pullable actuation elements 28, which in turn are coupled to and driven by one or more of the drive members 24. As shown in FIG. 1A, the pullable actuation elements 25 and 28 may extend distally and proximally from the force transmission assembly 23 through and/or along the shaft 21, and may be coupled to various parts of the instrument 1 such as to the movable component 20, the pushable actuation element 26, or the instrument shaft 21. In some embodiments, the pullable actuation elements 25 and 28 may pass along an exterior of the shaft 21 for a portion thereof and then may pass through an interior of the shaft 21 along another portion thereof, as illustrated in FIG. 1A.

One of the drive members 24 (also referred to as insertion drive member 24) is configured to drive an insertion degree of freedom of motion of the instrument (i.e., relative translation between the instrument shaft 21 and the force transmission assembly 23) by actuating two or more of the actuation elements 25. In particular, at least one of the proximally extending actuation elements 25 is coupled with the insertion drive member 24 and a proximal portion of the shaft 21 such that the insertion drive member 24 can drive translation of the shaft 21 in a distal direction relative to the transmission assembly 23 by drawing in this proximally coupled actuation element 25. In addition, at least one of the distally extending actuation elements 25 is coupled with the insertion drive member 24 and with a portion of the instrument that is distal of the force transmission assembly 23, such as a movable component 20, such that the insertion drive member 24 can drive translation of the shaft 21 in a proximal direction relative to the transmission assembly 23 by drawing in these distally coupled actuation elements 25. When the force transmission assembly 23 is in a fixed position relative to a workspace, such as mounted to a manipulator or otherwise held in a fixed position relative to a workspace, the above-noted relative translation results in translation of the shaft 21 relative to the fixed position at which the force transmission assembly 23 is coupled, which can be used for insertion or withdrawal of the end effector 22 relative to the workspace. As shown in FIG. 1B, in some embodiments the insertion drive member 24_1 comprises a drum 32 around which actuation elements 25 are at least partially wound, such that rotation of the insertion drive member 24_1 actuates the actuation elements 25. The drum 32 is coupled to one of the drive inputs 29, for example by a drive shaft 31. As shown in FIG. 1B, the pullable actuation elements 25 that are coupled to a distal end portion for the instrument shaft 21 (directly or via other components that are coupled to the instrument shaft 21) are wrapped around the drum 32 of the insertion drive member 24_1 in an opposite direction than the pullable actuation elements 25 that are coupled to the proximal end portion of the instrument shaft 21 (directly or via other components that are coupled to the instrument shaft 21), and therefore rotation of the drum 32 in one direction pays out the latter while simultaneously drawing in the former, and vice versa. In FIG. 1B, four pullable actuation elements 25 are shown that extend to the distal end portion of the instrument shaft 21 and one pullable actuation element 25 is shown that extends to the proximal end portion of the instrument shaft 21, but these are non-limiting examples and any number of pullable actuation elements 25 may be used as long as at least one pullable actuation element 25 is coupled to the distal end portion of the instrument shaft 21 and at least one is coupled to the proximal end portion of the instrument shaft 21.

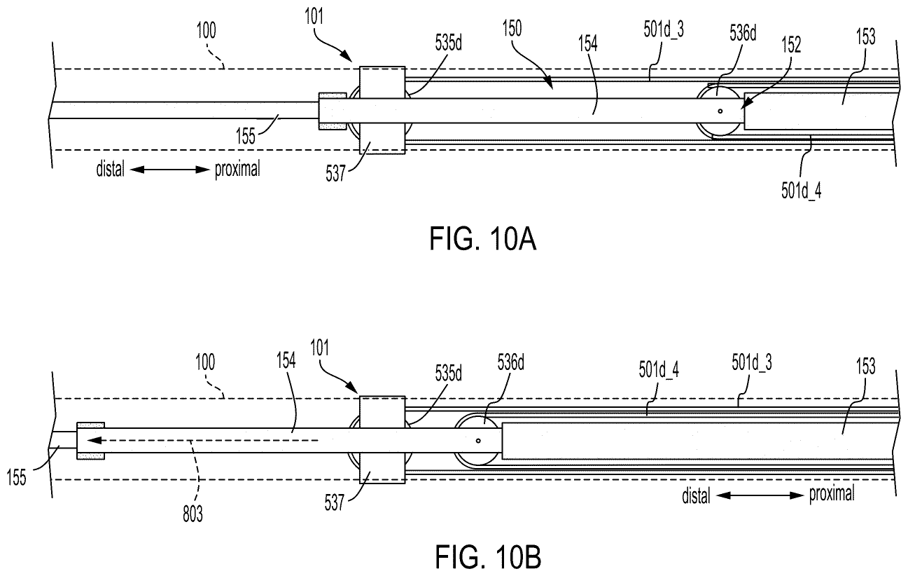

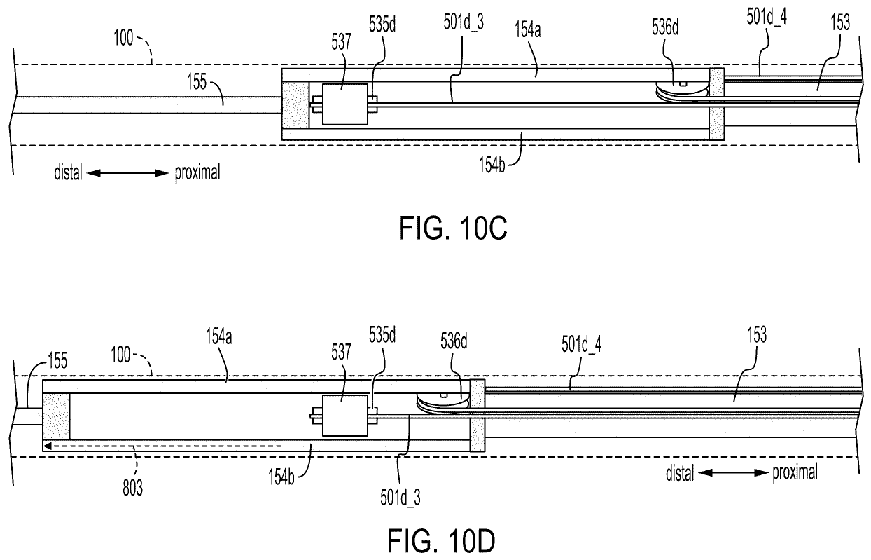

As shown in FIGS. 10-1D, one or more other drive members 24 (also referred to as non-insertion drive members 24) are configured to drive additional degrees of freedom of motion by actuating one or more respectively corresponding actuation elements 25 coupled thereto. In particular, FIG. 1C illustrates one example of a non-insertion drive member 24_2, which actuates pullable actuation elements 25 to drive motion of a movable component 20, while FIG. 1D illustrates a second example of a non-insertion drive member 24_3, which actuates pullable actuation elements 28 to drive translation of a pushable actuation element 26 relative to the instrument shaft 21 (which in turn drives motion of a movable component 20). The transmission assembly 23 may include either or both of these types of non-insertion drive members 24, in any number and combination. The transmission assembly 23 may also include other drive members or other functionality, as would be understood to those of ordinary skill in the art.

As noted above, FIG. 1C illustrates one type of non-insertion drive member 24_2 that drives motion of a movable component 20. The non-insertion drive member 24_2 comprises a drive shaft 31 coupled to at least one actuation element handling device 27. The actuation element handling device 27 is coupled to a corresponding actuation element 25 such that rotation of the drive shaft 31 actuates (draws in or pays out) the corresponding actuation element 25. A drive input 29 is coupled to the drive shaft 31 to drive rotation thereof. The drive input 29 may receive input driving forces, for example which may be received from a manipulator or from manual input. Moreover, in some embodiments, the non-insertion drive member 24_2 comprises multiple (e.g., 2 or more) of the actuation element handling devices 27 such that the drive member 24_2 can actuate multiple actuation elements 25 simultaneously. For example, in FIG. 1C the illustrated non-insertion drive member 24_2 has a pair of actuation element handling devices 27 and 27′ coupled to the same drive shaft 31, with the actuation elements 25_2 and 25_2′ being coupled to the actuation element handling devices 27 and 27′, respectively. In some embodiments, the actuation elements 25_2 and 25_2′ drive the same movable component 20 as one another along a same degree of freedom in opposite directions. For example, the actuation elements 25_2 and 25_2′ may be coupled to a same articulable structure 34 (e.g., joint) so as to articulate the articulable structure 34 about an axis in opposite directions. In some embodiments, the actuation elements 25_2 and 25_2′ are coupled to the actuation element handling devices 27 and 27′ in reversed configurations such that motion of the drive member 24_2 in one direction actuates the pair of actuation elements 25_2 and 25_2′ in opposite directions (e.g., rotation of the drive member 24_2 in one direction results in paying out one actuation element 25_2 and 25_2′ while simultaneously drawing in the other actuation element 25_2 and 25_2′, and vice versa for rotation of the drive member 24_2 in an opposite direction). In some embodiments, the actuation element handling devices 27 may be configured as actuation transfer devices, which are configured to allow an actuation element 25 coupled thereto to pass through the actuation element handling device 27 to also couple with the insertion drive member 24 such that the actuation element 25 can be independently actuated by both the insertion drive member 24 and the non-insertion drive member 24 of which the actuation element handling device 27 is a part. Thus, in some embodiments the actuation elements 25_2 and 25_2′ are coupled to and independently actuatable by both the insertion drive member 24_1 and the non-insertion drive member 24_2.

As noted above, FIG. 1D illustrates another type of non-insertion drive member 24_3 that drives motion of a pushable actuation element 26. The non-insertion drive member 24_3 comprises a capstan 33 coupled to a drive shaft 31 coupled to a drive input 29, such that rotation of the drive input 29 drives rotation for the capstan 33. In some embodiments, the drive shaft 31 is coupled directly with the drive input 29 as shown in FIG. 1D, while in other embodiments the drive shaft 31 is coupled indirectly with the drive input 29 via intermediaries, such as a gear mechanism described herein. The capstan 33 is coupled to at least one end portion of a pullable actuation element 28, which is at least partially wrapped around the capstan 33 such that rotation of the capstan 33 actuates the pullable actuation element 28. In some embodiments, two or more end portions of an actuation element 28 are coupled to the same capstan 33, as illustrated in FIG. 1D. The end portions may be wrapped around the capstan 33 in opposite directions as one another as shown in FIG. 1D, or they may be wrapped around the capstan 33 in the same direction as one another.

Turning now to FIG. 1E, the operation of the non-insertion drive members 24 comprising capstans 33, such as the non-insertion drive members 24_3 illustrated in FIG. 1D, will now be described. In some embodiments, just one non-insertion drive member 24 with a capstan 33 is used to drive the pushable actuation element 26, while in other embodiments two or more non-insertion drive members 24 with capstans 33 are used to drive the pushable actuation element 26. Regardless of the number of non-insertion drive members 24 used to drive the pushable actuation element 26, the one or more capstans 33 of the non-insertion drive members 24_3 are coupled (collectively) to two pullable actuation elements 28, which are labeled 28_3 and 28_4 in FIG. 1E. A first set of pulleys 35 is coupled to the instrument shaft 21 on opposite sides of the transmission assembly 23, including a first distal pulley 35d coupled to a distal portion of the instrument shaft 21 and a first proximal pulley 35p coupled to a proximal portion of the instrument shaft 21 as shown in FIG. 1E. A second set of pulleys 36 is coupled to the pushable actuation element 26 on opposite sides of the transmission assembly 23, including a second distal pulley 36d coupled to a distal portion of the actuation element 26 and a second proximal pulley 36p coupled to a proximal portion of the actuation element 26. The pullable actuation elements 28_3 and 28_4 are routed around the pulleys 35 and 36 so as to drive translation of the pushable actuation element 26—specifically, the actuation element 28_3 is routed around the second proximal pulley 36p and the first distal pulley 35d, while the actuation element 28_4 is routed around the second distal pulley 36d and the first proximal pulley 35p. Thus, drawing in the actuation element 28_3 while paying out the actuation element 28_4 causes translation of the actuation element 26 in the distal direction relative to the shaft 21, and paying out the actuation element 28_3 while drawing in the actuation element 28_4 causes translation of the actuation element 26 in the proximal direction relative to the shaft 21.

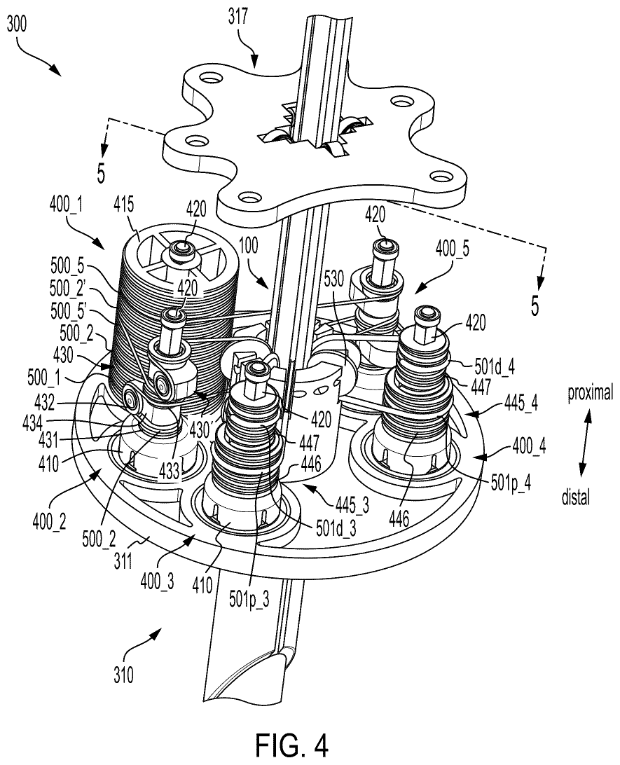

The actuation elements 28_3 or 28_4 may be drawn in by drawing in both ends thereof together (e.g., by one or more capstans such as the capstan 33), by drawing in one end thereof (e.g., by a capstan such as the capstan 33) while holding the other end thereof fixed (e.g., fixed to the transmission assembly 23), or by drawing in one end thereof at a first rate while paying out the other end thereof at a second, slower, rate (e.g., via a stepped capstan as described above, such as the capstans 445, 1445, or 4445 described below with reference to FIGS. 4, 13, and 20). Conversely, the actuation element 28_3 or 28_4 may be paid out by paying out both ends thereof together (e.g., by one or more capstans such as the capstan 33), by paying out one end thereof (e.g., a capstans such as the capstan 33) while holding the other end thereof fixed (e.g., fixed to the transmission assembly 23), or by paying out one end thereof at a first rate while drawing in the other end thereof at a second, slower, rate (e.g., via a stepped capstan as described above, such as the capstans 445, 1445, or 4445 described below with reference to FIGS. 4, 13, and 20). In various embodiments, the one or more capstan(s) 33 of the one or more non-insertion drive members 24 are coupled to the actuation elements 28_3 and 24_4 in a variety of configurations that allow the capstan(s) 33 to draw in and pay out the actuation elements 28_3 and 24_4 in one of the ways described above (e.g., by actuating both ends in the same direction, by actuating one end with the other fixed, or by actuating both ends in opposite directions at different rates) and thereby actuating the pushable actuation element 26.

Accordingly, the pushable actuation element 26 can be driven to translate relative to the shaft 21 by actuating the one or more non-insertion drive members 24_3 comprising the one or more capstans 33, as described above. In addition, the pushable actuation element 26 is configured to float relative to the transmission assembly 23 such that translation of the transmission assembly 23 relative to the shaft 21 does not affect the position of the actuation element 26 relative to the shaft 21, as will now be described in greater detail with reference to FIG. 1E. In various embodiments, when the instrument shaft 21 is driven to translate relative to the transmission assembly 23 via actuation of the insertion drive member 24_1, the second pair of pulleys 36 which are coupled to the instrument shaft 21 will move along with the instrument shaft 21. The one or more capstan(s) 33 may be held stationary while the instrument shaft 21 is being driven to translate, and therefore the paid-out length of pullable actuation elements 28_3 and 28_4 does not change while the insertion degree of freedom of motion is actuated. Because the paid-out length of pullable actuation elements 28_3 and 28_4 does not change while the pulleys 35 are being pulled to move along with the instrument shaft 21, resulting tension in one of the pullable actuation elements 28_3 and 28_4 causes the first pair of pulleys 36 to also move along with the instrument shaft 21, and because the first pair of pulleys 36 are coupled to the pushable actuation element 26 the motion of the first pair of pulleys 36 causes the pushable actuation element 26 to move along with the shaft 21. For example, when the insertion degree of freedom of motion is actuated to cause the instrument shaft 21 to move distally, the distal pulley 35d is pulled distally by the instrument shaft 21, which creates tension in the pullable actuation element 28_3 that pulls the pulley 36p to also move distally. The distally moving pulley 36p in turn pulls the actuation element 26 to also move distally. Conversely, when the instrument shaft 21 is moved proximally, the proximal pulley 35p is pulled proximally along with the instrument shaft 21, which creates tension in the pullable actuation element 28_4 that pulls the pulley 36d to move proximally, which in turn pulls the actuation element 26 to move proximally. Thus, the pushable actuation element 26 floats (is ungrounded) relative to the transmission assembly 23 and remains stationary (is grounded) relative to the instrument shaft 21 while the insertion degree of freedom of motion is actuated. In the explanation above it is assumed that the capstan(s) 33 are held stationary during actuation of the insertion degree of freedom of motion to simplify the description, but those having ordinary skill in the art would understand that the capstan(s) 33 could also be rotated while actuating the insertion degree of freedom of motion if desired, which will result in simultaneous translation of the instrument shaft 21 and actuation of the pushable actuation element 26 relative to the instrument shaft 21.

As noted above, the actuation elements 28_3 and 28_4 are coupled to and actuatable by one or capstans 33 of one or more drive members 24. In some embodiments two capstans 33 of two drive members 24 are used to actuate the actuation elements 28_3 and 28_4. For example, in some embodiments both ends of the actuation element 28_3 are coupled to one capstan 33 of one drive member 24, and both ends of the actuation element 28_4 are coupled to another capstan 33 of another drive member 24. In other embodiments, one end of the actuation element 28_3 is coupled to the capstan 33 of one drive member 24 and the other end is coupled to a fixed anchor point on the transmission assembly 23, and similarly one end of the actuation element 28_4 is coupled to the capstan 33 of another drive member 24 and the other end is coupled to a fixed anchor point on the transmission assembly 23. In the aforementioned embodiments with multiple capstans 33, the coordinated actuation of the capstans 33 drives actuation of the actuation element 26.

In other embodiments a single capstan 33 may drive both actuation elements 28_3 and 28_4. For example, in some embodiments both ends of both actuation elements 28_3 and 28_4 are coupled to the same capstan 33. In other embodiments, one end of each actuation element 28_3 and 28_4 is coupled to the same capstan 33, and the other end of each actuation element 28_3 and 28_4 is coupled to a fixed anchor point on the transmission assembly 23. In such single-capstan embodiments, the translation of the pushable actuation element 26 may be driven by a single drive member 24.

A person of ordinary skill in the art would understand that multiple distinct actuation elements 25 or 28 can be parts of the same larger body. Thus, for example, references herein to two actuation elements 28 (i.e., the actuation elements 28_3 and 28_4) being coupled to the pushable actuation element 26 should be understood as encompassing embodiments in which the two actuation elements 28 are two physically separate bodies and also embodiments in which the two actuation elements 28 are two physically conjoined portions of the same body (e.g., two segments of the same cable).

In some embodiments, the one or more capstan(s) 33 comprise stepped capstans having multiple steps with mutually different radii to which the end portions of the actuation elements 28 are coupled, to provide mechanical advantage, increased driving force, and different rates of drawing in/paying out, as described above. In some embodiments, the one or more capstan(s) 33 are not stepped capstans (i.e., the cable handling portions of the capstan all have the essentially the same radius). In some embodiments, the one or more capstan(s) 33 are coupled to drive inputs 29 via gear mechanisms that provide mechanical advantage and increased driving force, as described above. In some embodiments, the mechanical advantage provided by either the stepped capstans or the gear mechanisms may be arranged such that the one or more capstan(s) 33 are able to supply relatively strong driving forces to the pushable actuation element 26, including driving forces of at least 50 pounds (222 N) in some embodiments and at least 100 pounds (445 N) in some embodiments. The mechanical advantage can be tuned to a desired strength by appropriately setting the radii of the steps of the stepped capstan(s) (if used), wherein the smaller the difference in radii the more mechanical advantage is produced, or by appropriately setting the radii/tooth count of the gears of a gear assembly, wherein the greater the difference in radii/tooth count the more mechanical advantage is produced. Moreover, in some embodiments, the one or more capstan(s) 33 are configured to allow a relatively long range of motion for the pushable actuation element 26, including a range of motion of at least one inch (2.5 cm) in some embodiments and at least 2 inches (5.1 cm) in some embodiments.

Returning to FIG. 1A, in some embodiments the one or more movable components 20 of the instrument 1 include one or more articulable structure 34 (such as jointed links, flexible portions of a shaft, a wrist mechanism, etc.). Each articulable structure 34 has at least one corresponding degree of freedom of motion, which is driven by one or more actuation elements 25 coupled to the articulable structure 34. The articulable structure 34 can be used to couple the end effector 22 to the shaft 21 to allow for relative motion between the end effector 22 (or some other component) and the shaft 21, thereby allowing the pose (orientation relative to the shaft) of the end effector 22 to be changed. Such placement for an articulable structure 34 is nonlimiting and articulable structure 34 can also be used along the instrument shaft 21 to provide differing poses of portions of the shaft relative to other portions of the shaft 21, as those having ordinary skill in the art would be familiar with. In some embodiments, the articulable structure 34 provides differing degrees of freedom of motion. In some embodiments, multiple articulable structure 34 having differing degrees of freedom of motion are connected in series to form a wrist mechanism that couples the end effector 22 to the shaft 21 and to enable the end effector 22 to move with two or more degrees of freedom of motion (e.g., yaw, pitch, or combinations thereof) relative to the shaft 21. In some embodiments, the end effector 22 is coupled directly to the shaft 21 without an intervening articulable structure 34. References herein to an end effector being coupled to or supported by a shaft should be understood as broadly including both direct coupling and indirect coupling (e.g., via an articulable structure 34 or other intervening component), unless otherwise indicated or implied by the context. Although two articulable structure 34 comprising rotating joints are illustrated in FIG. 1A, it should be understood that this is not limiting and any number or type of articulable structure 34 could be used instead, including zero, one, three, or more articulable structure 34.

Moreover, in some embodiments, the one or more movable components 20 of the instrument 1 comprise movable components of the end effector 22 (in addition to, or in lieu of the articulable structure 34). In such embodiments, the movable components of the end effector 22 may be mechanically driven by driving forces transmitted to the end effector 22 from the drive members 24 via the pushable actuation element 26, as already described above and/or by flexible pullable actuation elements 25. Examples of such movable components of an end effector 22 may include a translatable staple firing component, a pivotable jaw member of a jaw mechanism, a translatable cutting element, etc. Such movable components of the end effector 22, when present, are instances of the above-noted movable components 20, and their motion may be considered a degree of freedom of motion of the instrument 1.

In some embodiments, the end effector 22 comprises a stapler comprising a jaw mechanism and a translatable staple firing component (sometimes referred to as a sled). In some embodiments, the pushable actuation element 26 drives the translatable staple firing component. In some embodiments, initial translation of the translatable staple firing component in a distal direction may cause the jaw mechanism of the stapler to close. In addition, further translation of the translatable staple firing component in the distal direction may fire staples from the jaw mechanism into a material, as a result of the staple firing component colliding with and pushing the staples out of the jaw mechanism. The closing of the jaw mechanisms may require relatively large driving forces due to possible resistance of the grasped material to being compressed between jaw members of the stapler. Similarly, the firing of the staples may require relatively large driving forces, due to friction and other forms of resistance such as resistance of the staples to being deformed and resistance of the grasped material to being pierced. However, in some embodiments, the pushable actuation element 26 and the drive member(s) 24 that drive the pushable actuation element 26 are able to provide sufficiently strong driving forces to actuate such a translatable staple firing component. Examples of stapler architectures that may be used as the end effector 22 and driven by the pushable actuation element 26 are disclosed in US Patent Application Publication No. 2019/0239967A1 and US Patent Application Publication No. 2019/0239877, the contents of each of which are incorporated herein by reference in their entirety.

The instrument 1 may also have additional degrees of freedom of motion. For example, in some embodiments the shaft 21 may be rotatable about its longitudinal axis, relative to the workspace, by rotation of the entire instrument 1 about the longitudinal axis. This may occur, for example, by rotation of an instrument holder to which the instrument 10 is mounted relative to a support that the instrument holder is coupled with (and hence rotation of the instrument 1 as a whole relative to the support). Such rotation of the instrument shaft 21 may be referred to as a roll degree of freedom of motion.

Although FIGS. 1A-1E illustrate embodiments having non-insertion drive members 24_2 of the type illustrated in FIG. 1C and non-insertion drive members 24_3 of the type illustrated in FIG. 1D, it should be understood that embodiments with different combinations of these parts are contemplated herein. In particular, embodiments contemplated herein include at least embodiments that have both one or more non-insertion drive members 24_2 and one or more non-insertion drive members 24_3, embodiments that have one or more non-insertion drive members 24_2 but no non-insertion drive members 24_3, and embodiments that have one or more non-insertion drive members 24_3 but no non-insertion drive members 24_2. Embodiments contemplated herein may also include additional types of drive members besides those illustrated in FIGS. 1A-1E. Other combinations of the various components are also contemplated herein, as would be apparent to a person of ordinary skill in the art.

### Second Instrument Embodiment

Turning now to FIGS. 2-10D, another embodiment of an instrument 10 is described below. The instrument 10 may be used as the instrument 1. The descriptions above of parts of the instrument 1 are applicable mutatis mutandis to the similar parts of the instrument 10.

As shown in FIG. 2, an embodiment of an instrument 10 comprises a shaft 100 supporting an end effector 210 at a distal end portion of the shaft 100 (proximal and distal directions referenced herein are illustrated in FIG. 2). The end effector 210 is configured to perform one or more functions, similar to the end effector 22 as described above. The instrument 10 also comprises one or more movable components 200, similar to the movable components 20 described above, coupled to a distal end portion of the shaft 100. The one or more movable components 200 may comprise one or more components of the end effector 210 (e.g., a jaw member of a jaw mechanism, a translating component such as a cutting component or a staple firing mechanism, etc.) and/or one or more articulable structures 230 coupling the end effector 210 (or some other component of the instrument 10) to the shaft 100 and/or provided along the shaft 100 to impart articulation to the shaft 100. The instrument 10 also comprises a force transmission assembly 300, similar to the force transmission assembly 23 described above, which is movably coupled to the shaft 100 proximal of the movable components 200 (the shaft 100 and force transmission assembly 300 move relative to one another, as described below, and thus the coupling location of the force transmission assembly 300 to the shaft 100 varies, but the force transmission assembly 300 remains positioned generally proximal of the movable component(s) 200 of the instrument 10, and in the case of a medical instrument, the force transmission assembly 300 remains outside of a body in which the instrument is inserted to perform a procedure).

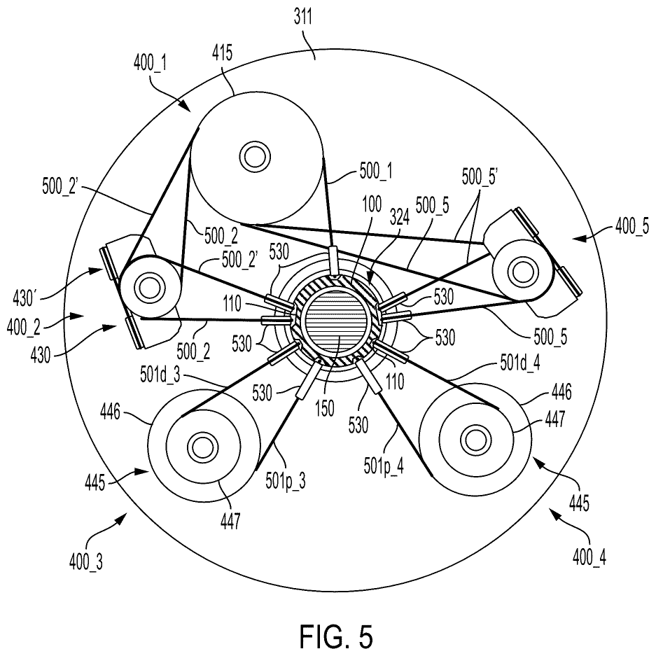

The force transmission assembly 300 may be used as the force transmission assembly 23 and comprises drive members 400 (described in greater detail below in relation FIG. 5) configured to receive input driving forces, with the driving forces controlling degrees of freedom of motion of the instrument 10. In various embodiments, the force transmission assembly 300 is mountable to a manipulator of a computer-controlled, teleoperated system (such as the system 1000 described in further detail below), and the input driving forces are provided to the drive members 400 through the manipulator interface (as also describe in further detail below). The interface may instead be configured to receive input driving forces manually. The drive members 400 may be used as the drive members 24 described above. The drive members 400 convert and transfer the driving forces to actuation elements. Specifically, the instrument 10 comprises a first set of pullable actuation elements 500, a second set of pullable actuation elements 501, and a pushable actuation element 150. The flexible pullable actuation elements 500 and 501 may be used as the actuation elements 25 and 28, respectively, and the pushable actuation element 150 may be used as the pushable actuation element 26 described above. FIGS. 4-10D, which are described in greater detail below, illustrate the flexible pullable actuation elements 500 and 501 and the pushable actuation element 150.

The flexible pullable actuation elements 500 may also be referred to herein as “pullable actuation elements 500” or “actuation elements 500,” and similarly the flexible pullable actuation elements 501 may also be referred to herein as “pullable actuation elements 501” or “actuation elements 501.” The actuation elements 500 and 501 may comprise tension members capable of transmitting pulling (tensioning) forces while also being relatively flexible in lateral directions perpendicular to a longitudinal dimension of the member, such as, for example, cables, wires, filaments, belts, chains, straps, ropes, etc. Such pullable actuation elements may also be referred to in the art as tension members or pull-pull actuation elements. The actuation elements 500 are distinguished from the actuation elements 501 in that the actuation elements 500 are coupled to drive members 400_2 and 400_5 that comprise actuation transfer mechanisms 430, whereas the actuation elements 501 are coupled to drive members 400_3 and 400_4 that comprise capstans 445. The drive members 400_2 and 400_5 may be used as the drive member 24_2 described above, while the drive members 400_3 and 400_4 may be used as the drive member 24_3 described above. The actuation elements 500 are also distinguished from the actuation elements 501 in that the actuation elements 500 are coupled to the insertion drive member 400_1 (which may be used as the insertion drive member 24_1 described above), whereas the actuation elements 501 are not coupled to the insertion drive member 400_1. In some embodiments, the actuation elements 500 may be similar in structure to the actuation elements 501, while in other embodiments the actuation elements 500 may have a different structure than the actuation elements 501. For example, in some embodiments, the actuation elements 501 may be stronger than the actuation elements 500, for example by virtue of being thicker and/or made of a more resilient material, which may be beneficial in embodiments in which the actuation elements 501 are subjected to larger driving forces than the actuation elements 500.

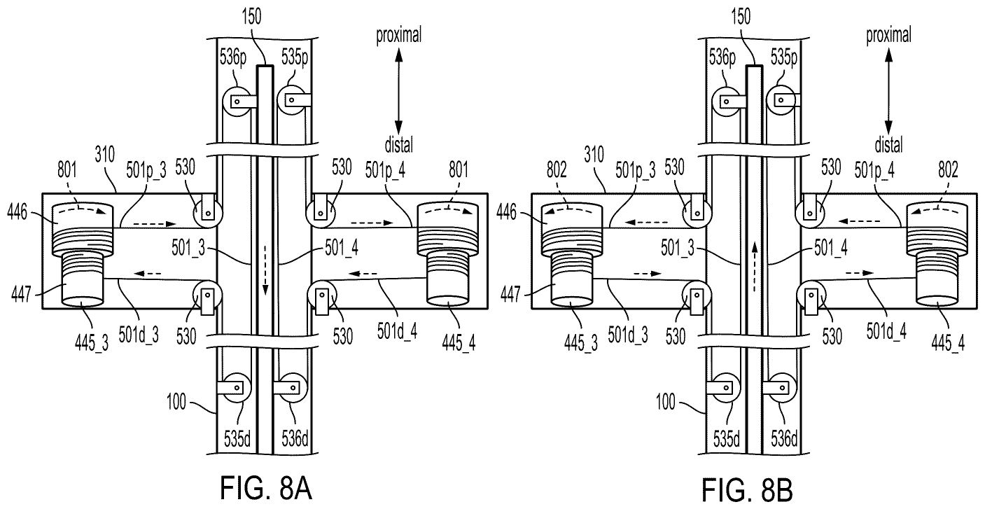

The pullable actuation elements 500 extend through and/or along the shaft 100 and are coupled to the movable components 200 and/or other parts of the instrument 10 to transmit the driving forces so as to drive various degrees of freedom of motion of the instrument 10, which may include degrees of freedom of motion of the movable components 200 as well as additional degrees of freedom of motion. For example, some of the actuation elements 500 are coupled to and drive motions of articulable structures 230 (such as jointed links, flexible portions of a shaft, etc.), each of which has at least one corresponding degree of freedom of motion. Other actuation elements 500 may be coupled to the shaft 100, for example to drive an insertion degree of freedom of motion. The pullable actuation elements 501 also extend through and/or along the shaft 100 and are coupled to and drive the pushable actuation element 150 (see FIGS. 8A and 8B), which then in turn is coupled to a movable component 200.

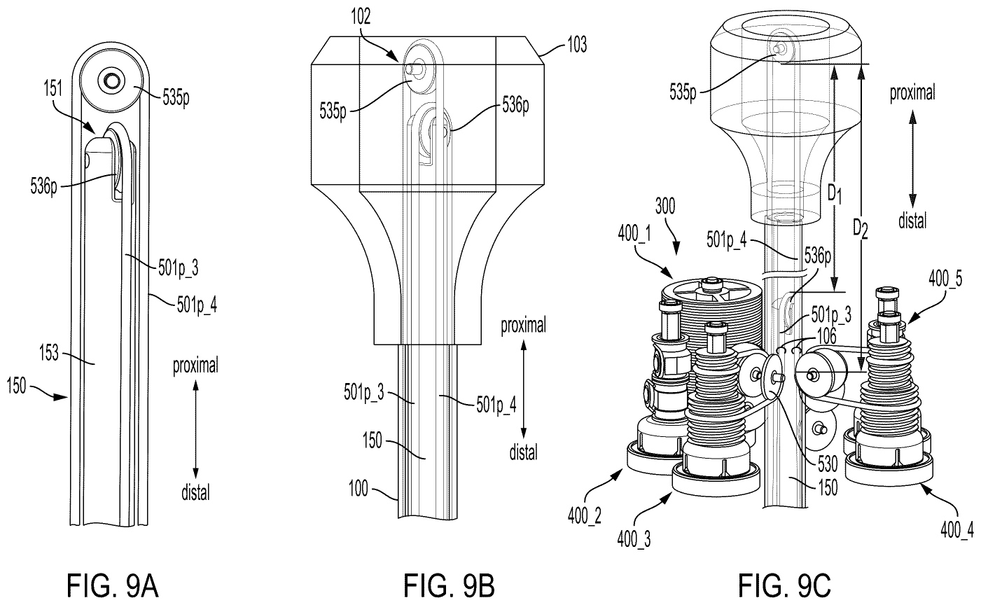

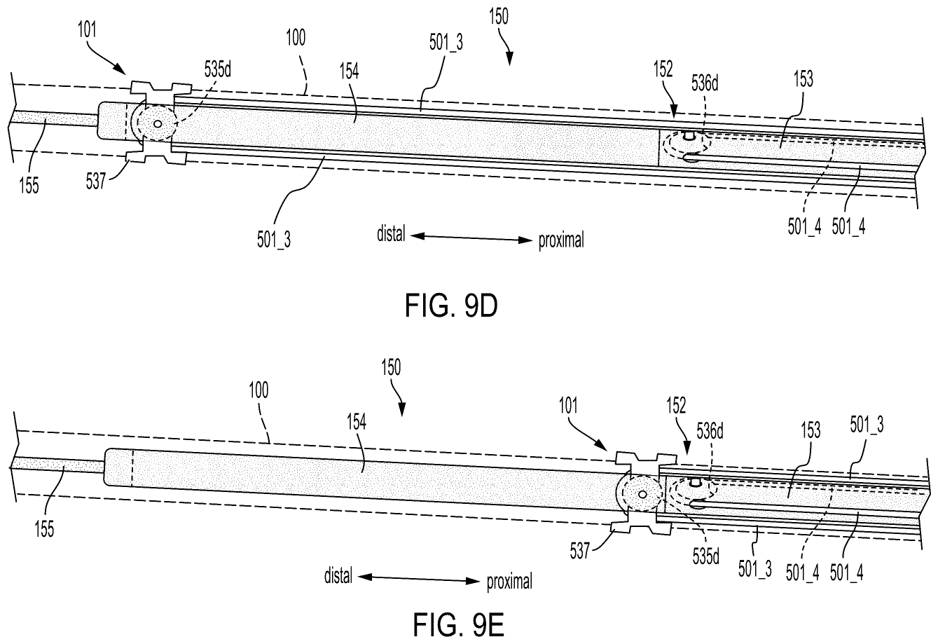

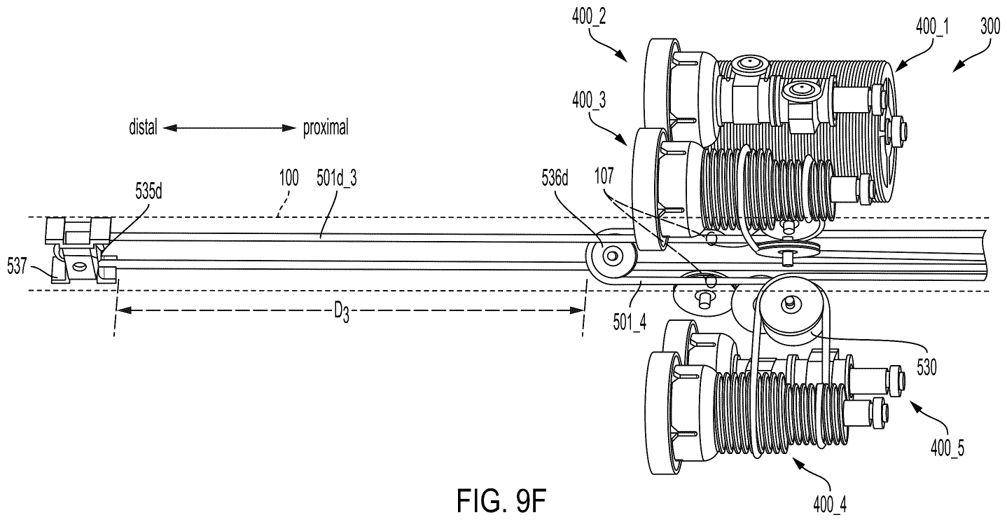

The pushable actuation element 150 may also be referred to herein as an “actuation element 150”. The pushable actuation element 150 may comprise a compression member or an assembly of members capable of withstanding compressive forces and transmitting pushing forces. For example, the pushable actuation element 150 may comprise one or more push-pull members, which are relatively rigid axially and capable of transmitting both pushing and pulling forces, such as, for example, tubes, rods, bars, beams, etc. As another example, the pushable actuation element 150 may comprise one or more laterally flexible push-pull members, which are relatively rigid axially and capable of transmitting both pushing and pulling forces while also being laterally flexible in one or more dimensions, such as a flexible pullable member that is laterally surrounded by a supporting structure to prevent buckling (e.g., a push cable), or a sheet or beam that is thin in one lateral dimension so that the beam is flexible along the thin dimension while being relatively rigid along its other dimensions, etc. As another example, the pushable actuation element 150 may comprise one or more laterally flexible push-push members, which are relatively rigid axially and thus capable of transmitting pushing forces while also being flexible in multiple lateral directions, such as, for example, coil pipes, cut tubes (e.g., laser cut tubes), etc. Such laterally flexible push-push members are distinguished from the laterally flexible push-pull members in that the push-push members may not be well suited to transmitting pulling forces, as they may stretch under tension, whereas push-pull members may be well suited for both pushing and pulling. Laterally flexible push-pull or laterally flexible push-push members may be particularly useful in embodiments with an articulable structure 230 (e.g., joint), as such members can bend with the articulable structure 230 and retain the ability to transfer axial pushing forces even while bent. Thus, in some embodiments, the actuation element 150 may have a multi-part structure with one or more parts comprising a more rigid push-pull type member and one or more parts comprising a more flexible push-pull or push-push type member. In particular, in some embodiments at least a portion of the actuation element 150 that passes through an articulable structure is configured as a flexible push-push or push-pull type member, such as a beam that is flexible in at least one dimension, a coil pipe, or the like. FIGS. 9A-10D, which are described in greater detail below, illustrate one example embodiment of the actuation element 150.

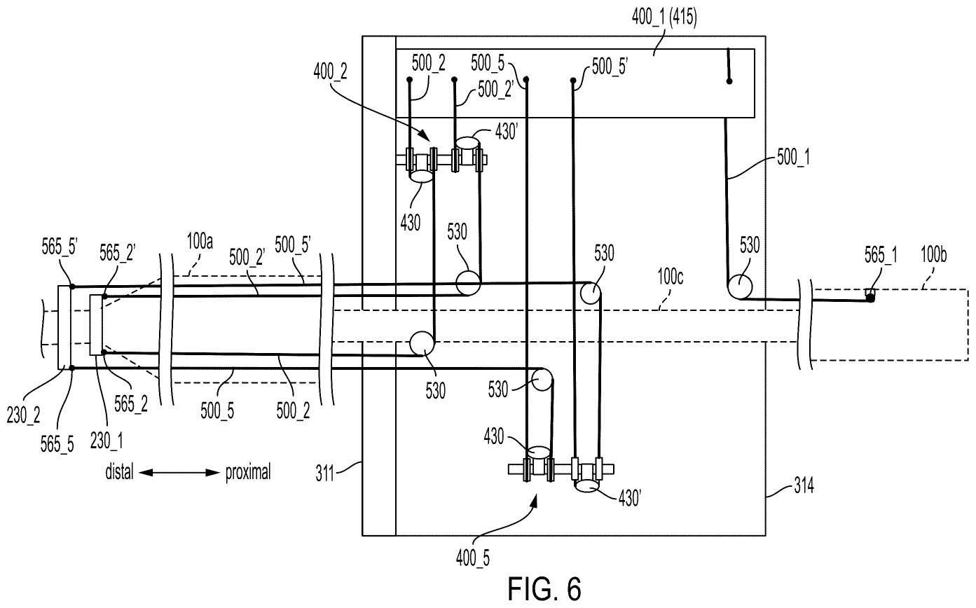

As noted above, some portions of the actuation elements 500 and 501 may be routed inside the instrument shaft 100 and other portions of the actuation elements 500 and 501 may be routed external to the instrument shaft 100. More specifically, as shown schematically in FIG. 6, in some embodiments the actuation elements 500 may be routed inside of the instrument shaft 100 while passing through a distal portion 100a or a proximal portion 100b of the shaft 100, and the actuation elements 500 may be routed along an exterior of the instrument shaft 100 while traversing an intermediate portion 100c of the shaft 100, the intermediate portion 100c being located between the distal and proximal portions 100a and 100b. The intermediate portion 100c may comprise a portion of the instrument shaft 100 along which the transmission assembly 300 translates. In some embodiments, while passing along the exterior of the instrument shaft 100, the actuation elements 500 may be routed through grooves 110 in an exterior surface of the instrument shaft 100, as shown in FIG. 5. The actuation elements 500 may transition from the exterior of the instrument shaft 100 to an interior thereof via openings (not illustrated) in and through an outer wall of the instrument shaft 100. Similar to the actuation elements 500, the actuation elements 501 may be routed external to the instrument shaft in the intermediate portion 100c and internal to the instrument shaft in the distal and proximal portions 100a and 100b.

Components of the force transmission assembly 300 and other associated parts of the instrument 10 are described below in greater detail with reference to FIGS. 2-10D. For organizational purposes and to assist in readability, the description includes subheadings for the various components that will be described. However, it should be understood that while in some embodiments, the various components are used together, other embodiments of force transmission assemblies may not necessarily include all the components discussed.

### Force Transmission Assembly Housing

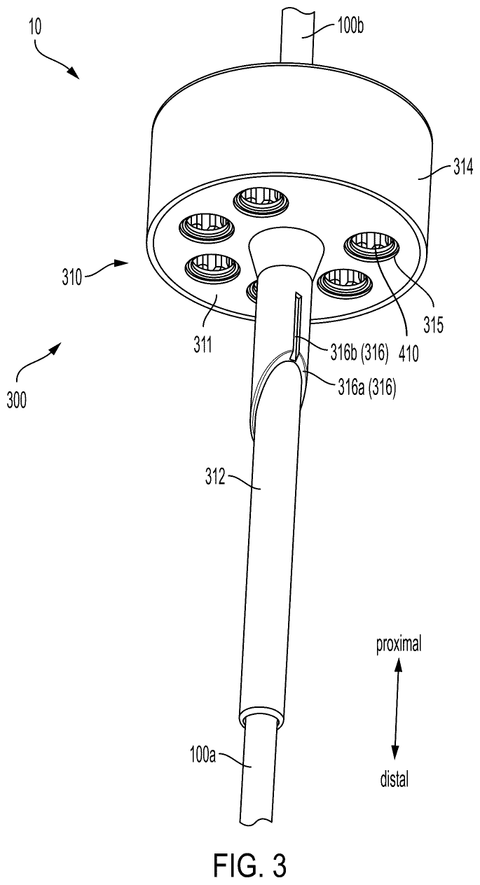

As shown in FIGS. 2 and 3, the force transmission assembly 300 comprises a housing 310. The housing 310 comprises a chassis 311, a sleeve 312 extending distally from the chassis 311, and a cover portion 314. The chassis 311 serves as a platform or a base that supports other components of the force transmission assembly 300, such as the drive members 400 described in further detail below. The chassis 311 and the cover portion 314 cooperate together to define a chamber in the housing 310 within which the drive members 400 and other components of the force transmission assembly 300 are housed.

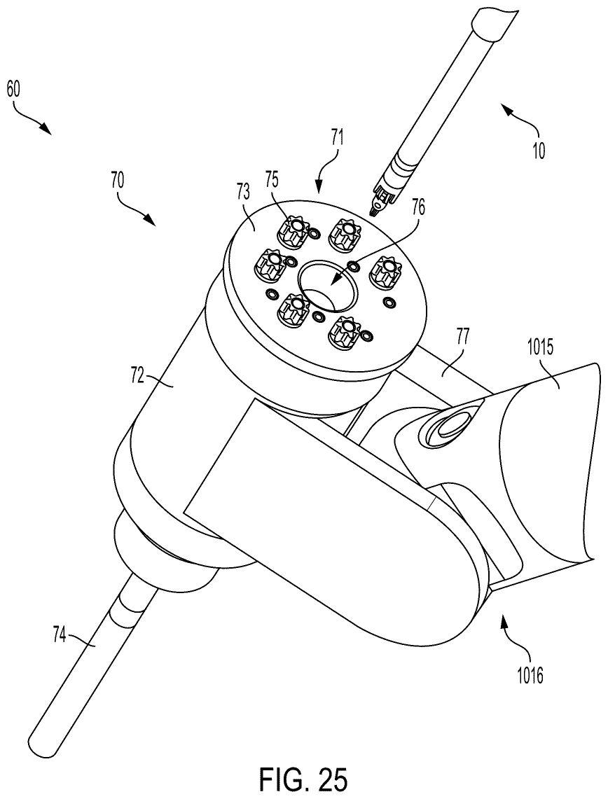

In the embodiment of FIGS. 2-10D, the chassis 311 also provides an interface for coupling the instrument 10 to a manipulator. Specifically, a distal face of the chassis 311, visible in FIG. 3, forms an interface for the instrument 10 that is mountable to a complementary interface of the manipulator (e.g., interface 73, described below with respect to FIG. 25). The chassis 311 may engage either directly with the manipulator, or the chassis 311 may engage indirectly with the manipulator via an intermediary device, such as a sterile adaptor that is interposed between the manipulator and the instrument 10 to provide a sterile barrier. As shown in FIG. 3, the distal face of the chassis 311 comprises openings 315 through which drive inputs 410 of the drive members (visible in FIGS. 4, 9C, and 9F) are accessible from an exterior of the chassis 311, for example from the distal side of the chassis 311 in the depicted embodiment. Corresponding drive outputs of the manipulator to which the instrument 10 is mounted (e.g., drive outputs 75, described below with respect to FIG. 25) may thus interact with the drive inputs 410 via the openings 315 to transfer driving forces to the drive inputs 410. The drive outputs of the manipulator may engage directly with the drive inputs 410, or they may be engaged indirectly via an intermediary, such as intermediate couplers of the sterile adaptor. The drive inputs 410 and the drive outputs (or intermediate couplers) may have complementary engaging surfaces, such as engaging surfaces with complementary groves and protrusions or other complementary keying features, such that when the drive inputs 410 are engaged with the drive outputs or intermediate couplers they are constrained to rotate together. In FIG. 3, the drive inputs 410 are shown as having receptacles into which complementary drive outputs or intermediate couplers are received, but in other embodiments, the drive inputs 410 may comprise protrusions configured to be received within complementary receptacles of the drive outputs or intermediate couplers, or any of a variety of other mating engagements to allow the drive inputs to engage with the corresponding drive outputs or intermediate couplers. In FIG. 3, the drive inputs 410 are shown as being wholly contained within the housing 310, but in other embodiments the drive inputs 410 may extend distally from the chassis 311 through the openings 315.

Referring again to FIGS. 2 and 3, a sleeve 312 of the housing 310 surrounds the instrument shaft 100 and extends distally from the chassis 311. The sleeve 312 is configured to be insertable into a passage of a manipulator when the instrument 10 is mounted to the manipulator (e.g., described further below with reference to FIG. 25). The sleeve 312 facilities sealing of the workspace to prevent escape of pressurized fluid. The sleeve 312 may also comprise alignment features 316 to aid in alignment of the instrument 10 relative to the manipulator as the instrument 10 is mounted to the manipulator. For example, FIG. 3 illustrates two alignment features 316, namely first alignment feature 316a and second alignment feature 316b. The first alignment feature 316a comprises an angled surface portion forming a shoulder or series of shoulders extending circumferentially around the sleeve 312 and configured to engage with complementary surfaces of the manipulator as the sleeve 312 is inserted into the passage of the manipulator to provide progressively increasing alignment between the instrument 10 and the instrument holder the further the instrument 10 is inserted into the passage. The second alignment feature 316b may comprise one or more straight notches extending proximally from an apex of the first alignment feature 316a and generally parallel to a longitudinal axis of the instrument shaft 100. The second alignment feature 316b interacts with a complementary feature of the manipulator once the instrument 10 has been inserted sufficiently far into the passage. The first alignment feature 316a is configured such that they will bring the instrument 10 into an aligned orientation by the time the instrument 10 has advanced sufficiently into the passage for the second alignment features 316b to be engaged. The second alignment feature 316b then holds the instrument 10 in the aligned orientation as the instrument 10 continues to be advanced into the passage until a mounted position is reached. The initial alignment forced by the first alignment feature 316a may bring the drive inputs 410 into alignment with the corresponding drive outputs of the manipulator prior to coupling thereof, and then the continued advancement of the instrument thereafter while the second alignment feature 316b maintains the aligned orientation may enable the coupling between the drive inputs 410 and drive outputs to be completed.

In the illustrated embodiment, the sleeve 312 and the chassis 311 are integrally coupled together, or in other words they are both part of the same monolithic body. In other embodiments, the sleeve 312 and the chassis 311 are separately formed parts that are coupled together, for example, by mechanical fasteners, friction fitting, adhesive, welding, or other known joining techniques.

### Drive Members-Overview

Turning now to FIG. 4, embodiments of drive members 400 and other related components of the transmission assembly 300 will be described in greater detail. FIG. 4 comprises a perspective view of the transmission assembly 300, with the cover portion 314 removed to show the drive members 400. The drive members 400 may be used as the drive members 24 described above.

As shown in FIG. 4, the transmission assembly 300 comprises a plurality of drive members 400. The drive members 400 each actuate (i.e., pay out or draw in) one or more actuation elements 500 or 501, which drives a corresponding degree of freedom of motion of the instrument 10. As shown in FIG. 4, some drive members 400 may actuate two or more actuation elements 500 or 501. As noted above, the pullable actuation elements 501 are coupled to and actuate the pushable actuation element 150, and thus the drive members 400 that drive those actuation elements 501 may also be said to actuate the pushable actuation element 150. In the illustrated embodiment, the transmission assembly 300 comprises five drive members 400, with a first drive member 400_1 being configured to drive an insertion degree of freedom of motion and the other drive members 400_2 to 400_5 being configured to drive at least one other degree of freedom of motion of the instrument 10. The drive member 400_1 may also be referred to herein as an “insertion drive member,” and the other drive members 400 may also be referred to as “non-insertion drive members.” Although four non-insertion drive members 400_2 to 400_5 are illustrated, it should be understood that any number of non-insertion drive members equal to or greater than one may be included.

As noted above, each drive member 400 drives a corresponding degree of freedom of motion of the instrument 10 by paying out or drawing in at least one corresponding actuation element 500 or 501. In particular, as shown in FIG. 4, each drive member 400 comprises a drive shaft 420 about which the drive member 400 is rotatable relative to the chassis 311, with rotation of the drive member 400 about its drive shaft 420 causing the corresponding actuation elements 500 or 501 to pay out or draw in, depending on the direction of rotation. Paying out or drawing in one of the actuation elements 500 or 501 may also be referred to herein as “actuating” the actuation element 500 or 501 when the particular direction of motion (i.e., paying out or drawing in) is not specified. Similarly, causing the drive shaft 420 of a drive member 400 to rotate may also be referred to herein as “actuating” the drive member 400. Thus, using this nomenclature, a given actuation element 500 or 501 may be actuated by actuating a corresponding drive member 400.

As shown in FIGS. 3 and 4, each drive member 400 also comprises a drive input 410 coupled to the drive shaft 420 such that the drive shaft 420 is constrained to rotate with the drive input 410. As noted above, in a mounted state of the instrument 10 to a manipulator, the drive input 410 receives driving forces from a drive output of the manipulator, and these driving forces drive rotation of the drive shaft 420 coupled to the drive input 410. (The driving forces may also be referred to as torques, because they induce rotational motion; herein, references to “force” are not limited to forces urging linear motion, and can include torques that urge rotational motion.) As shown in FIG. 4, a distal end portion of each drive shaft 420 is coupled to a drive input 410, which is rotatably coupled to the chassis 311. In addition, a proximal end portion of each drive shaft 420 may be rotatably coupled to a common proximal support 317 (illustrated in dashed lines for ease of viewing). Thus, each drive shaft 420 has one rotational bearing at a proximal end portion thereof and another rotational bearing at the distal end portion thereof in the form of the chassis 311 (via the drive inputs 410).

In addition to the drive input 410 and the drive shaft 420, each drive member 400 also comprises one or more actuation element handling components that couple the drive member 400 to at least one corresponding actuation element 500 or 501. The actuation element handling components of each drive member 400 are coupled to the drive shaft 420 thereof such that rotation of the drive shaft 420 causes the actuation element component to actuate the corresponding actuation element(s) 500 coupled thereto. In the case of the insertion drive member 400_1, the actuation element handling component comprises a rotatable drum 415 (hereinafter drum 415). In the case of the non-insertion drive members 400_2 and 400_5, their respective actuation element handling components comprise actuation transfer mechanisms 430. Specifically, in the embodiment illustrated in FIG. 4, each non-insertion drive member 400_2 to 400_5 comprises two actuation transfer mechanism 430 and 430′ that are coupled to corresponding actuation elements 500. The actuation transfer mechanisms 430 may be used as the actuation element handling device 27 described above. In the case of the non-insertion drive members 400_3 and 400_4, their respective actuation element handling components comprise capstans 445. Specifically, in the embodiment illustrated in FIG. 4, the capstans 445 comprise stepped capstans that have multiple stepped portions with different radii. These actuation element handling components and their interactions with the actuation elements 500 are described in greater detail below.

### Insertion Drive Member and Corresponding Actuation Elements

With reference to FIGS. 4-6, an embodiment of an insertion drive member 400_1 will be described in greater detail below. FIG. 5 comprises a partial sectional plan taken from the perspective along 5-5 in FIG. 4. FIG. 6 is a schematic diagram showing how actuation elements 500 are routed and connected to other components of the instrument, such as the drive members 400 of the force transmission assembly 300. The diagram in FIG. 6 is not intended to show actual sizes, actual shapes, actual or relative physical locations of components, or the actual paths taken by actuation elements 500, but instead FIG. 6 shows the connections and routing schematically and in simplified form to aid understanding. It should be understood that additional handling components besides those illustrated, such as pulleys, could be interposed in the actuation element paths, and in actual implementations the actuation element paths may deviate from those shown. Moreover, in FIG. 6 the instrument shaft 100 is indicated schematically by dashed lines, and different portions of the shaft 100 are shown with different widths to facilitate illustration of the disposition of the actuation elements 500 relative to the shaft 100 in and along different portions thereof, but it should be understood that, as with other figures, the illustrated dimensions of the shaft 100 are not meant to be accurate or to scale. In FIG. 6, the drive members 400_3 and 400_4, as well as the actuation element 501 driven thereby, are omitted from the figure to improve clarity because these components do not affect the insertion degree of freedom of motion.

As noted above, the insertion drive member 400_1 comprises a rotatable drum 415 to actuate (i.e., draw in and pay out) the actuation elements 500 that are coupled to the drum 415. The term “drum” as used herein is intended to refer broadly to any component having a rotational axis and a bearing surface extending generally circumferentially (although not necessarily in a perfect circle) around the rotational axis around which flexible pullable actuation elements 500 (such as cables, wires, filaments, chains, straps, belts, etc.) can be wound by rotation of the drum 415. The drum 415 may also be referred to as, for example, a cylinder, a spool, a capstan, a windlass, a winch, or the like. The drum 415 may have guide elements, such as grooves and/or ridges, to guide actuation elements 500 as they wind around the drum 415, or the drum 415 may omit such guide elements and have a generally smooth surface.

Multiple pullable actuation elements 500 are coupled to and wound around drum 415 such that rotation of the drum 415 draws in or pays out the actuation elements 500, depending on direction of rotation. As shown in FIGS. 4-6, one or more of the actuation elements 500 that are coupled to the drum 415 are also coupled to and actuatable by a non-insertion drive member 400. But, as will be described in more detail below, these actuation elements 500 can move past or through the non-insertion drive members 400 to which they are coupled when the actuation elements 500 are actuated by the drum 415 (due to the actuation transfer mechanisms 430, described below); therefore, the non-insertion drive members 400 can be ignored when considering the operation of the insertion drive member 400_1.

At least one of the actuation elements 500 coupled to the drum 415 (e.g., actuation element 500_1) extends proximally from the transmission assembly 300 and is coupled (directly or indirectly) to a proximal portion 100b of the shaft 100 at a position proximal of the force transmission assembly 300, as shown schematically in FIG. 6. At least one other actuation element 500 coupled to the drum 415 (e.g., actuation elements 500_2, 500_2′, 500_5, and 500_5′) extends distally from the force transmission assembly 300 and is coupled (directly or indirectly) to a distal portion 100a of the shaft 100 at a position distal of the force transmission assembly 300, as shown schematically in FIG. 6. As shown in FIGS. 4 and 6, each actuation element 500 that is coupled to the proximal portion 100b of the instrument shaft 100 (e.g., actuation element 500_1) is wound around the drum 415 in an opposite direction than the actuation elements 500 that are coupled to the distal portion 100a of the shaft 100 (e.g., actuation elements 500_2, 500_2′, 500_5, and 500_5′). Therefore, rotation of the drum 415 in one direction draws in the actuation element(s) 500 that are coupled to the proximal portion 100b (e.g., actuation element 500_1) while simultaneously paying out the actuation elements 500 coupled to the distal portion 100a (e.g., actuation elements 500_2, 500_2′, 500_5, and 500_5′), and rotation of the drum 415 in an opposite direction has the opposite effect. The drawing in of the actuation element(s) 500 coupled to the proximal portion 100b pulls the instrument shaft 100 to move in a distal direction relative to the transmission assembly 300. Conversely, the drawing in of the actuation elements 500 coupled to the distal portion 100a pulls the instrument shaft 100 to move in a proximal direction relative to the transmission assembly 300. Thus, the insertion drive member 400_1 is actuatable to control the insertion degree of freedom of motion of the instrument 10, or in other words to drive relative motion of the shaft 100 and the force transmission assembly 300.

As noted above, in the embodiment illustrated in FIGS. 2-10D, the one or more actuation elements 500 that are coupled to the proximal portion 100b of the shaft 100 comprise the actuation element 500_1. In some embodiments, the actuation element 500_1 is not coupled to any of the non-insertion drive members 400. As shown in FIGS. 5 and 6, the actuation element 500_1 is routed from the drum 415 to a waterfall pulley 530 that has an axis of rotation perpendicular to the axis of rotation of the drum 415. The waterfall pulley 530 redirects the actuation element 500_1 to extend proximally along the shaft 100. The actuation element 500_1 extends along the shaft 100, for example via a groove 110 (see FIG. 5) in an outer surface of the shaft 100. As shown schematically in FIG. 6, the actuation element 500_1 extends proximally until it reaches a coupling location 565_1 at which the actuation element 500_1 is coupled either directly or indirectly to the shaft 100. The coupling location 565_1 may be part of the shaft 100 or part of some other component that is itself coupled (directly or indirectly) to the shaft 100. The coupling location 565_1 may be located somewhere along the proximal portion 100b of the shaft 100, for example at a position that remains proximal of the transmission assembly 300 throughout a range of motion of the shaft 100 and transmission assembly 300 relative to one another. Thus, when the distal insertion actuation element 500_1 is drawn in, it pulls on the shaft 100 and urges the shaft 100 to move in the distal direction.

As noted above, in the embodiment illustrated in FIGS. 2-10D, the actuation elements 500 that are coupled to the distal portion 100a of the shaft 100 comprise actuation elements 500_2, 500_2′, 500_5, and 500_5′. In this embodiment, all of these actuation elements 500_2, 500_2′, 500_5, and 500_5′ are coupled to both the drum 415 and to one of the non-insertion drive members 400_2 and 400_5. Specifically, as shown in FIGS. 5 and 6, the actuation elements 500_2, 500_2′, 500_5, and 500_5′ have respective proximal end portions coupled to the drum 415, extend from the drum 415 to couple with actuation transfer mechanisms 430 (described below) of the drive members 400_2 and 400_5, extend from the drive members 400_2 and 400_5 to corresponding waterfall pulleys 530, and then extend from the waterfall pulleys 530 distally along and through the shaft 100 to the distal portion 100a of the shaft 100 where respective distal end portions of the actuation elements 500_2, 500_2′, 500_5, and 500_5′ are coupled to coupling points 565_2, 565_2′, 565_5, and 565_5′, respectively, as shown in FIG. 6. The coupling points 565_2, 565_2′, 565_5, and 565_5′ may be coupled directly or indirectly with the distal portion 100a of the shaft 100. In the embodiment in FIGS. 2-10D the coupling points 565_2, 565_2′, 565_5, and 565_5′ are part of one or more articulable structures 230, which are coupled to the shaft 100.

Specifically, in the embodiment illustrated in FIG. 6, a first pair of actuation elements 500_2 and 500_2′ are coupled to coupling points 565_2 and 565_2′ that are part of a first articulable structure 230_1, and a second pair of actuation elements 500_5 and 500_5′ are coupled to coupling points 565_5 and 565_5′ that are part of a second articulable structure 230_2. In other embodiments, the coupling points 565_2, 565_2′, 565_5, and 565_5′ may all be part of the same articulable structure 230, which may comprise one or more joints. In other embodiments, some or all of the coupling points 565_2, 565_2′, 565_5, and 565_5′, and 565_4′ may be parts of other movable component 200 other than an articulable structure 230, parts of the shaft 100, or parts of some other component of the instrument 10 which is coupled to the shaft 100, such as any moveable components that may utilize a coordinated pair of pull-pull actuation elements to move the moveable component.

In the embodiment of FIG. 6, the coupling points 565_2, 565_2′, 565_5, and 565_5′ and the one or more articulable structures 230 to which they are coupled may be configured such that, when all of the actuation elements 500_2, 500_2′, 500_5, and 500_5′ are simultaneously drawn in at the same rate, then the one or more articulable structures 230 are locked in whatever their current position is by equal and opposite tension applied thereto from opposing pairs of the actuation elements 500_2, 500_2′, 500_5, and 500_5′. Thus, when all of the actuation elements 500_2, 500_2′, 500_5, and 500_5′ are simultaneously drawn in, the one or more articulable structures 230 do not articulate and instead a net force in the proximal direction is applied to the articulable structures 230, and hence also to the shaft 100 to which they are coupled, resulting in proximal translation of the shaft 100. Because the actuation elements 500_2, 500_2′, 500_5, and 500_5′ are all wrapped around the drum 415 in a same direction, rotation of the drum 415 in one direction (e.g., clockwise in FIG. 4) draws in all of the actuation elements actuation elements 500_2, 500_2′, 500_5, and 500_5′ simultaneously, resulting in proximal translation of the shaft 100 along the insertion axis relative to the transmission assembly 300.

Thus, the actuation element 500_1 drives the insertion degree of freedom of motion along one direction when the insertion drive member 400_1 is rotated in a first direction to draw in the actuation element 500_1, and the actuation elements 500_2, 500_2′, 500_5, and 500_5′ collectively drive the insertion degree of freedom of motion along an opposite direction when the insertion drive member 400_1 is rotated in a second direction to draw in the actuation elements 500_2, 500_2′, 500_5, and 500_5′.

During actuation of the insertion degree of freedom of motion, the pushable actuation element 150 floats relative to the transmission assembly and remains stationary relative to the instrument shaft 100. This is important to ensure that actuation of the insertion degree of freedom of motion does not inadvertently cause actuation of movable components that are driven by the actuation element 150, such as a component of the end effector 210. The actuation element 150 is held stationary relative to the shaft 100 while the shaft 100 is driven to move relative to the transmission assembly 300 by the actuation elements 501 coupled to the actuation element 150 and by pulleys 536 and 535 that route the actuation elements 501 (described in greater detail below with reference to FIG. 9A), as described above with respect to the actuation element 26.

In some embodiments, the transmission assembly 300 may include another actuation element (not illustrated), which is dedicated for driving the proximal translation of the shaft 100 along the insertion axis and not for driving any other movable components 200. For example, such an actuation element may be coupled to the drum 415 and extend distally from the force transmission assembly 300 to couple directly to a distal portion 100a of the shaft 100, such that pulling in the actuation element 500 causes proximal translation of the shaft 100.

### Non-Insertion Drive Members with Actuation Transfer Mechanisms

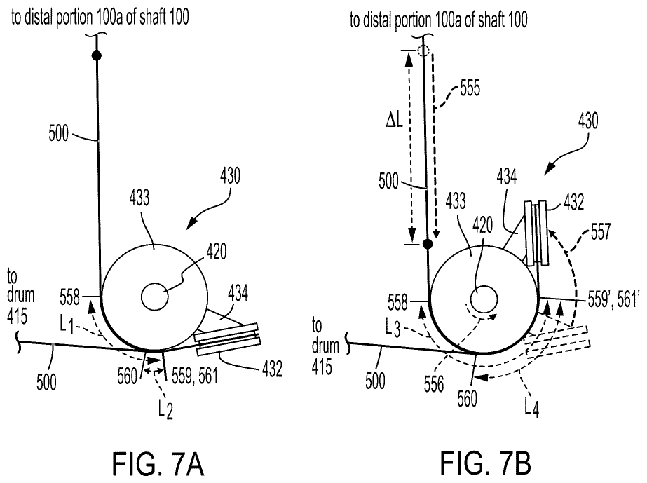

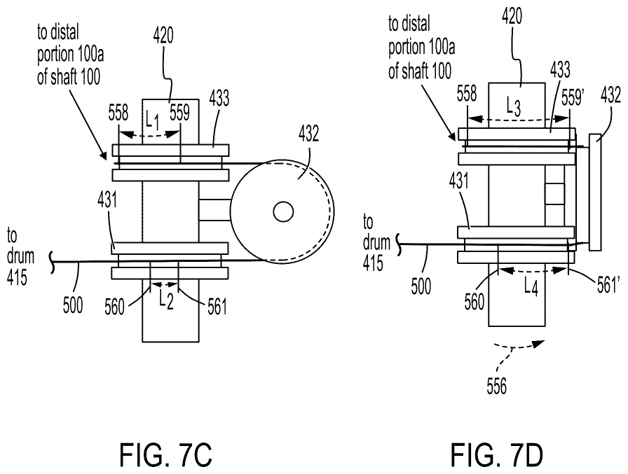

With reference to FIGS. 4-7D, the non-insertion drive members 400 that have actuation transfer mechanism 430 will be described in greater detail below. FIGS. 7A and 7B illustrate plan views of an actuation transfer mechanism 430 (described in greater detail below) in two states from a perspective above (i.e., proximal of) the actuation transfer mechanism 430. FIGS. 7C and 7D illustrate side views of the actuation transfer mechanism 430 in the states of FIGS. 7A and 7B, respectively.

As noted above, at least some of the non-insertion drive members 400 comprise one or more actuation transfer mechanisms 430 to couple with and actuate actuation elements 500, as shown in 4-7D. Specifically, with reference to FIGS. 4-6, the non-insertion drive members 400_2 and 400_5 comprise two actuation transfer mechanisms 430 each. For example, the drive member 400_2 comprises actuation transfer mechanism 430 and 430′, both coupled to the same drive shaft 420 of the drive member 400_2, and likewise for the non-insertion drive member 400_5.

An actuation transfer mechanism 430 of a given drive member 400_2 or 400_5 is configured to allow both the insertion drive member 400_1 and the given non-insertion drive member 400_2 or 400_5 to actuate the same actuation element 500 independently of one another. In other words, if the drum 415 is held stationary while a given non-insertion drive member 400_2 or 400_5 is rotated, the actuation transfer mechanism 430 of the given non-insertion drive member 400_2 or 400_5 actuates (pays out or draws in) the corresponding actuation element cable 500. Likewise, if the drum 415 is rotated while the given non-insertion drive member 400_2 or 400_5 is held stationary, the actuation transfer mechanism 430 allows the drum 415 to actuate the actuation element 500. Thus, the actuation transfer mechanism 430 can allow a transfer of actuation of the same actuation element 500 between the drum 415 and the corresponding non-insertion drive member 400_2 or 400_5 to which the actuation element 500 is coupled.

As shown in FIGS. 4 and 7A-7D, in some embodiments, an actuation transfer mechanism 430 comprises a first pulley 431 rotatably coupled to and coaxial with the drive shaft 420, a second pulley 432 radially offset from the drive shaft 420 and having an axis of rotation oriented transverse to an axis of rotation of the drive shaft 420, and a third pulley 433 rotatably coupled to and coaxial with the drive shaft 420. The second pulley 432 is attached to an arm member 434 coupled to and extending generally radially from the drive shaft 420 and constrained to rotate with the drive shaft 420.

The manner in which the actuation elements 500 couple with the actuation transfer mechanisms 430 is described below using the actuation transfer mechanism 430 of the drive member 400_2, as shown in FIGS. 4 and 5, as an example. The same principles apply to the other actuation transfer mechanisms 430 and their respectively corresponding actuation elements 500 and non-insertion drive members 400. As shown in FIGS. 4 and 5, the actuation element 500_2 is routed from the drum 415 to the first pulley 431 such that the actuation element 500_2 begins to wrap around the first pulley 431 and hence also the drive shaft 420 in a first direction, then the actuation element 500_2 is looped around the second pulley 432 and redirected back to the third pulley 433 to wrap around third pulley 433 and hence the drive shaft 420 again in a second direction opposite the first direction. From the third pulley 433, the actuation element 500_22 extends to a waterfall pulley 530, which redirects the actuation element 500_3 to extend along the shaft 100.

The actuation transfer mechanisms 430 as described above allow the actuation elements 500 coupled thereto to move past or through the non-insertion drive members 400 when actuated by the insertion drive member 400_1. For example, if the actuation element 500_2 is drawn in or paid out by the insertion drive member 400_1, the pulleys 431, 432, and 433 of the actuation transfer mechanism 430 of the drive member 400_2 rotate around their respective axes in response to motion of the actuation element 500_2, thereby allowing the actuation element 500_2 to be paid out or drawn in through the actuation transfer mechanism 430 without causing or requiring actuation (rotation) of the drive member 400_2. Thus, though the drive member 400_2 is held stationary, the actuation element 500_2 can nevertheless be drawn in or paid out, for example by the insertion drive member 400_1. In other words, the actuation transfer mechanism 430 allows the insertion drive member 400_1 to actuate (pay in or draw out) the actuation element 500_2 independently of actuation of the drive member 400_2. The same is true of the other actuation transfer mechanisms 430 and their respectively corresponding actuation elements 500. Thus, the actuation transfer mechanisms 430 allow the insertion drive member 400_1 to actuate all of the actuation elements 500 coupled thereto simultaneously to drive the insertion degree of freedom of motion independently of any actuation of the drive members 400_2 or 400_5.

On the other hand, the actuation transfer mechanism 430 as described above also allows the non-insertions drive members 400 to actuate their respectively corresponding actuation elements 500, notwithstanding the fact that the actuation elements 500 are free to move through the actuation transfer mechanisms 430. Returning to the actuation transfer mechanism 430 of the drive member 400_2 as an example, when the drive shaft 420 of the drive member 400_2 is rotated, this causes the arm 434 of the actuation transfer mechanism 430 to also rotate with the shaft 420, which in turn causes the second pulley 432 coupled to the arm 434 to revolve around the drive shaft 420. Because the actuation element 500_2 is looped around the second pulley 432, the revolution of the second pulley 432 around the drive shaft 420 pays out or draws in the actuation element 500_2, depending on the direction of motion. Specifically, revolution of the second pulley 432 around the drive shaft 420 in one direction, increases the amount of the actuation element 500_2 that is wound around the first and third pulleys 431 and 433, thus drawing in the actuation element 500_2. Conversely, revolution of the second pulley 432 around the drive shaft 420 in an opposite direction decreases the amount of the actuation element 500_2 that is wound around the first and third pulleys 431 and 433, thus drawing in the actuation element 500_2. Thus, the drive member 400_2 can actuate the actuation element 500_2 even if the insertion drive member 400_1 is held stationary. The same is true of the other actuation transfer mechanisms 430 and their respectively corresponding actuation elements 500. Thus, the actuation transfer mechanisms 430 allow the non-insertion drive members 400_2 and 400_5 to independently actuate their corresponding actuation elements 500, independently of the insertion drive member 400_1. Such independent actuation can in turn transfer forces to cause movement of a corresponding moveable component of the instrument to which the actuation elements are operably coupled.

The manner in which a non-insertion drive member 400 actuates an actuation element 500 via the actuation transfer mechanism 430 can be better understood by considering FIGS. 7A-7D, which illustrate schematically and in greater detail an actuation transfer mechanism 430. FIGS. 7A and 7B illustrate the actuation transfer mechanism 430 in a plan view from a perspective proximal of the actuation transfer mechanism 430, while FIGS. 7C and 7D illustrate a side view of the actuation transfer mechanism 430. In FIGS. 7A and 7C, the actuation transfer mechanism 430 is in a first state, and in FIGS. 7B and 7D the actuation transfer mechanism 430 is in a second state, in which the drive shaft 420 has been rotated counterclockwise (in the view of the figure) relative to the first state, as indicated by the arrow 556. The rotation of the drive shaft 420 causes the second pulley 432 to revolve counterclockwise around the drive shaft 420, as indicated by the arrow 557 in FIGS. 7B and 7D. In the first state shown in FIGS. 7A and 7C, the portion of the actuation element 500 that is wound around the third pulley 433 has a length L₁ measured from the location where the actuation element 500 initially contacts the third pulley 433 (location 558) to the location where the actuation element 500 terminates contact with the third pulley 433 (location 559) to extend to the second pulley 432. In addition, the portion of the actuation element 500 that is wound around the first pulley 431 has a length L₂ measured from the location where the actuation element 500 initially contacts the first pulley 431 (location 560) to the location where the actuation element 500 terminates contact with the first pulley 431 to extend to the second pulley 432 (location 561). In the second state shown in FIGS. 7B and 7D (after rotation of the drive shaft 420), the portion of the actuation element 500 that is wound around the third pulley 433 has a length L₃ measured from the location where the actuation element 500 initially contacts the third pulley 433 (location 558) to the location where the actuation element 500 terminates contact with the third pulley 433 to extend to the second pulley 432 (location 559′). In addition, the portion of the actuation element 500 that is wound around the first pulley 431 has a length L₄ measured from where the actuation element 500 initially contacts the first pulley 431 (location 560) to where the actuation element 500 terminates contact with the first pulley 431 to extend to the second pulley 432 (location 561′). As can be seen by comparing the lengths L₁ and L₂ illustrated in FIG. 7A with the lengths L₃ and L₄ illustrated in FIG. 7B, the rotation of the drive member 400 about the drive shaft 420 increases the total length of the actuation element 500 that is wound around the first and third pulleys 431 and 433. Specifically, the total length of the actuation element 500 that is wound around the first and third pulleys 431 and 433 in the first state is L₁+L₂, while in the second state it is L₃+L₄, where L₄>L₂ and L₃>L₁. Thus, the total length of the actuation element 500 that is wound around the first and third pulleys 431 and 433 has increased by an amount ΔL=L₃+L₄−(L₁+L₂) between the first and second states. Because one end portion of the actuation element 500 is attached to the drum 415 and thus held stationary, the other end portion of the actuation element 500, which is attached to a distal portion 100a of the shaft 100 (e.g., via a movable component 200), will be drawn in by the distance ΔL to provide for the increased length of the actuation element 500 wrapped round the first and third pulleys 431 and 433. The opposite would occur if the drive shaft 420 were rotated in the opposite direction (clockwise in the view of FIGS. 7A-7B), i.e., the length of the actuation element 500 wound around the pulleys 431 and 433 would shorten, resulting in the actuation element 500 paying out.

In the embodiment illustrated in FIGS. 4-7D, the pulleys 431, 432, and 433 are rotatable around their axes of rotation, which may reduce friction and makes actuation of the actuation elements 500 easier. However, in some embodiments, one, some, or all of the pulleys 431, 432, and 433 could be replaced with non-rotating bearings. Operation of the actuation transfer mechanism 430 would be the same in such an embodiment, except that the actuation element 500 would slide relative to the bearings rather than the pulleys 431, 432, and 433 rotating. This may result in increased friction, but in some circumstances this may be acceptable.

As can be seen in FIGS. 4-6, the actuation transfer mechanisms 430 and 430′ of the same drive member 400 have reversed configurations relative to one another, meaning that they are coupled to the drive shaft 420 of the drive member 400 such that rotation of the drive shaft 420 in one direction causes the actuation transfer mechanisms 430 and 430′ to actuate their corresponding actuation elements 500 and 500 in opposite directions, i.e., paying out one while drawing in the other, and vice versa. The actuation transfer mechanisms 430 and 430′ of a same drive member 400 may be arranged in the reversed configuration by, for example, positioning the respective second pulleys 432 thereof to face in the same direction as one another when their respective arms 434 extend in opposite directions from one another, as shown in FIG. 4, or in other words, by positioning the respective second pulleys 432 on opposite sides of their respective arms 434 so as to face in opposite directions as one another when their respective arms 434 are aligned in the same direction.

As already noted above, in some embodiments, the actuation elements 500_2, 500_2′, 500_5, and 500_5′ are coupled to one or more articulable structures 230, and when all are drawn in simultaneously, these actuation elements 500 lock the articulable structures 230 and drive translation (insertion/retraction) of the shaft 100. However, when some of these actuation elements 500_2, 500_2′, 500_5, and 500_5′ are drawn in while others are simultaneously paid out, then the articulable structures 230 are driven to articulate along one or more degrees of freedom of motion, while the shaft 100 is held stationary.

For example, with reference again to FIG. 6, a first articulable structure 230_1 is coupled to actuation elements 500_2 and 500_2′ such that simultaneous actuation of the actuation elements 500_2 and 500_2′ in opposite directions drives movement of the first articulable structure 230_1 along a degree of freedom of motion (e.g., yaw). Thus, in this embodiment, actuation of the drive member 400_2 can drive motion of the articulable structure 230_1 along a degree of freedom of motion, because actuation of the drive member 400_2 results in simultaneous actuation of the actuation elements 500_2 and 500_2′ in opposite directions, as described above. Similarly, in this embodiment, the second articulable structure 230_2 is coupled to the actuation elements 500_5 and 500_5′ such that simultaneous actuation of the actuation elements 500_5 and 500_5′ in opposite directions drives movement of the second articulable structure 230_2 along a degree of freedom of motion (e.g., pitch). Thus, in this embodiment, actuation of the drive member 400_5 can drive motion of the articulable structure 230_2 along a degree of freedom of motion, because actuation of the drive member 400_5 results in simultaneous actuation of the actuation elements 500_5 and 500_5′ in opposite directions, as described above.

In other embodiments, instead of each drive member 400_2 and 400_5 driving separate degrees of freedom of motion of the articulable structures 230, the actuation elements 500_2, 500_2′, 500_5, and 500_5′ may be coupled to the articulable structures 230 such that coordinated actuation of both drive member 400_2 and 400_5 is used to actuate either degree of freedom of motion. For example, instead of having two pairs of actuation elements 500 each coupled to a different articulable structure 230 having a single degree of freedom of motion, with the actuation elements 500 of a pair being coupled to opposite sides of their respective articulable structure 230, in some embodiments all four actuation elements 500_2, 500_2′, 500_5, and 500_5′ may be coupled to the same articulable structure 230 which has multiple degrees of freedom of motion. In such an arrangement, different types of motion of the articulable structure 230 may be obtained based on the combination of which of the actuation elements 500_2, 500_2′, 500_5, and 500_5′ are drawn in and which are paid out.

### Non-Insertion Drive Members with Capstans

With reference to FIGS. 4, 5, and 8, the non-insertion drive members 400 that utilize capstans 445 will be described in greater detail below. FIGS. 8A and 8B are schematic illustrations of routing of the actuation elements 501 between capstans 445 (e.g., capstans 445_3 and 445_4) and the pushable actuation element 150. Like the schematic illustration of FIG. 6, the schematic illustrations of FIGS. 8A and 8B show the routing schematically and in simplified form but are not intended to show actual paths of the actuation elements 501 or the relative positions, orientations, or sizes of parts—in particular, in FIGS. 8A and 8B, the capstans 445 are illustrated in an orientation that permits a simplified illustration of the actuation elements 501 without crossed paths, but an actual orientation of the capstans 445 may be different.

As noted above, at least some of the non-insertion drive members 400 comprise capstans 445 to couple with and actuate actuation elements 501, as shown in FIGS. 4, 5, and 8A-8B. Specifically, in the embodiment of FIGS. 4, 5, and 8A-8B, the non-insertion drive members 400_3 and 400_4 comprise capstans 445_3 and 445_4, respectively, which are coupled with the actuation elements 501_3 and 501_3, respectively. In other embodiments, only one drive member comprises a capstan. In yet other embodiments, more than two drive members comprise a capstan. As shown in FIG. 4, the capstan 445 is coupled to the drive shaft 420 of the corresponding non-insertion drive member 400, such that rotation of the drive shaft 420 (driven by rotation of the drive input 410) causes rotation of the capstan 445, thereby actuating (drawing in or paying out) the corresponding actuation element 501. The term “capstan” as used herein is intended to refer broadly to any component having a rotational axis and a bearing surface extending generally circumferentially (although not necessarily in a perfect circle) around the rotational axis around which flexible pullable actuation element 501 (such as cables, wires, filaments, chains, straps, belts, etc.) can be wound by rotation of the capstan. The capstan may also be referred to as, for example, a drum, a cylinder, a spool, a capstan, a windlass, a winch, or the like.

Each capstan 445 comprises one or more actuation element winding portions, such as the actuation element winding portions 446 and 447 illustrated in FIGS. 4, 5, and 8A-8B, around which an actuation element 501 is wound (wrapped) by rotation of the capstan. The actuation element winding portions 446 and 447 of the capstans 445 may optionally comprise grooves or other guide elements to guide the actuation elements 501 as they wind onto or off from the capstan 445. In the embodiment illustrated in FIGS. 4, 5, and 8A-8B, the capstans 445 are stepped capstans and thus the actuation element winding portions 446 and 447 therefore are configured as steps that have mutually different radii (i.e., the actuation element winding portions 446 has a larger radius than the actuation element winding portions 447 in the illustrated embodiment). In other embodiments, the capstans are not stepped and have one or more actuation element winding portions that have the same radius as one another, as described further below.

In the embodiments illustrated in FIGS. 4, 5, 8A, and 8B, each capstan 445 is coupled to both end portions of a single corresponding actuation element 501, with one end portion being coupled to the actuation element winding portion 446 and the other end portion being coupled to the actuation element winding portion 447. For example, as shown in FIG. 8A, the actuation element 501_3 has an end portion 501p_3 coupled the actuation element winding portion 446 of the capstan 445_3 and another end portion 501d_3 coupled to the actuation element winding portion 447 of the same capstan 445_3, with the actuation element 501_3 forming a loop between these two end portions via the waterfall pulleys 530 and the pulleys 536p and 535d. Similarly, as shown in FIG. 8A, the actuation element 501_4 has an end portion 501p_4 coupled the actuation element winding portion 446 of the capstan 445_4 and another end portion 501d_4 coupled to the actuation element winding portion 447 of the same capstan 445_4, with the actuation element 501_4 forming a loop between these two end portions via the waterfall pulleys 530 and the pulleys 536d and 535p. The pulleys 535 and 536 will be described in greater detail below. As used herein, an end portion of the actuation element 501 refers to a segment of the actuation element 501 that extends between one end thereof and one of the pulleys 536 or 535. The end portions of a given actuation element 501 may be referred to herein using the characters p and d appended to the reference label 501, with p indicating that the end portion extends between one end and a proximal pulley 535p or 536p and d indicating that the end portion extends between one end and a distal pulley 535d or 536d. Thus, for example, the end portion 501p_3 refers to the end portion of the actuation element 501 that extends between one end of the actuation element 501_3 and the proximal pulley 536p.

As shown in FIG. 8A, the pulleys 535 are coupled to the instrument shaft 100, whereas the pulleys 536 are coupled to the pushable actuation element 150. More specifically, the pulleys 535 comprise a pulley 355p coupled to the instrument shaft 100 at a position that is proximal of the transmission assembly 300 throughout its range of motion and a pulley 535d coupled to the instrument shaft 100 at a position that is distal of the transmission assembly 300 through its range of motion. The pulleys 535 may be coupled directly or indirectly to the instrument shaft 100, but regardless of how the pulleys 535 are coupled with the instrument shaft 100, the pulleys 535 are axially fixed (stationary) relative to the instrument shaft 100. The pulleys 536 comprise a pulley 356p coupled to the actuation element 150 at a position that is proximal of the transmission assembly 300 throughout its range of motion and a pulley 535d coupled to the actuation element 150 at a position that is distal of the transmission assembly 300 through its range of motion. The pulleys 536 may be coupled directly or indirectly to the actuation element 150, but regardless of how they are coupled with the actuation element 150, the pulleys 536 are axially fixed (stationary) relative to the actuation element 150.

In view of the above-described configuration of the pulleys 535 and 536 and how the actuation elements 501_3 and 501_4 are coupled thereto, actuation of the actuation elements 501_3 and 501_4 can drive the actuation element 150 to translate distally or proximally relative to the instrument shaft 100. In particular, if the actuation element 501_3 is drawn in on the capstan 445_3, the loop portion of the actuation element 501_3 is shortened, thereby creating a force tending to pull the proximal pulley 536p and the distal pulley 535d towards one another. Because the distal pulley 535d is axially fixed relative to the instrument shaft 100, the force pulling the pulleys 536p and 535d toward one another is translated into a force tending to pull the proximal pulley 536p to move distally relative to the instrument shaft 100. The proximal pulley 536p is coupled to the actuation element 150, and thus the force pulling the proximal pulley 536p distally relative to the instrument shaft 100 is translated into a force moving the actuation element 150 distally relative to the instrument shaft 100. Thus, if the actuation element 501_3 is drawn in, this supplies a force to the actuation element 150 tending to move it distally relative to the instrument shaft 100. Motion of the actuation element 150 distally relative to the instrument shaft 100 will result in the pulleys 535p and 536d moving further apart from one another, and therefore the actuation element 501_4 will resist the distal motion of the actuation element 150 unless the actuation element 501_4 is paid out. Accordingly, distal translation of the actuation element 150 may be achieved by simultaneously drawing in the actuation element 501_3 while paying out, from the capstan 445_4, the actuation element 501_4. Conversely, translation of the actuation element 150 in the proximal direction relative to the instrument shaft 100 may be achieved by simultaneously paying out the actuation element 501_3 while drawing in the actuation element 501_4, for reasons similar to those described above.

In the embodiment illustrated in FIGS. 4, 5, and 8A-8B, the drawing in of the actuation elements 501_3 is effectuated by rotating the capstan 445_3 in a first direction, while the paying out of the actuation elements 501_3 is effectuated by rotating the capstan 445_3 in a second direction. As shown in FIGS. 4, 5, and 8A, the two end portions 501p_3 and 501d_3 of the actuation element 501_3 are wrapped around the capstan 445_3 in opposite directions, and therefore when the capstan 445_3 is rotated in a given direction, the two end portions 501p_3 and 501d_3 will be actuated in opposite directions from one another, i.e., one end portion 501p_3 will pay out while the other end portion 501d_3 is drawn in, and vice versa. If the actuation element 501_3 were coupled to a non-stepped capstan, the above-described reversed actuation of the ends of the actuation element 501_3 would prevent the paid out length of the actuation element 501_3 from being changed when the capstan was rotated, as equal lengths of the actuation element 501_3 would be simultaneously paid out and drawn in. However, because the capstans 445 are stepped capstans as illustrated in FIGS. 4, 5, 8A, and 8B, the actuation element winding portions 446 and 447 have different radii and therefore the rates at which the two end portions 501p_3 and 501d_3 pay out or are drawn in when the capstan 445 is rotated will be different. Specifically, the end portion 501p_3 will always be paid out or drawn in at a faster rate than the end portion 501d_3 because the actuation element winding portion 446 has a larger radius than the actuation element winding portion 447. The foregoing is also true for the actuation element 501_4 and the capstan 445_4 for similar reasons.

Accordingly, as shown in FIG. 8A, when the capstans 445_3 and 445_4 are rotated in a first direction 801, the overall actuation element 501_3 is paid out (because the end portion 501p_3 is paid out faster than the end portion 501d_3 is drawn in) while the overall actuation element 501_4 is drawn in (because the end portion 501p_4 is drawn in faster than the end portion 501d_4 is paid out), resulting in the actuation element 150 being forced to move distally. Conversely, as shown in FIG. 8B, when the capstans 445_3 and 445_4 are rotated in a second direction 802, the overall actuation element 501_3 is drawn in (because the end portion 501p_3 is drawn in faster than the end portion 501d_3 is drawn in) while the actuation element 501_4 is paid out (because the end portion 501p_4 is paid out faster than the end portion 501d_4 is drawn in), resulting in the actuation element 150 being forced to move proximally.

The above described configuration of the capstans 445 as stepped capstans and the opposite wrapping directions of the actuation elements 501 on capstans 445 produces a mechanical advantage, increasing the force that can be transferred to the actuation element 150 for a given amount of torque supplied to the capstan 445. In particular, the smaller that the non-zero difference is between the radii of the actuation element winding portions 446 and 447, the larger the mechanical advantage will be. In other words, as the difference in radii gets smaller (down to but not including zero), the number of revolutions of the capstan 445 that are needed to change the paid-out length of the actuation element 501 by a given amount increases, thus increasing the mechanical advantage. The increased mechanical advantage may be advantageous in applications where it is desirable for the pushable actuation element 150 to supply relatively large pushing forces.

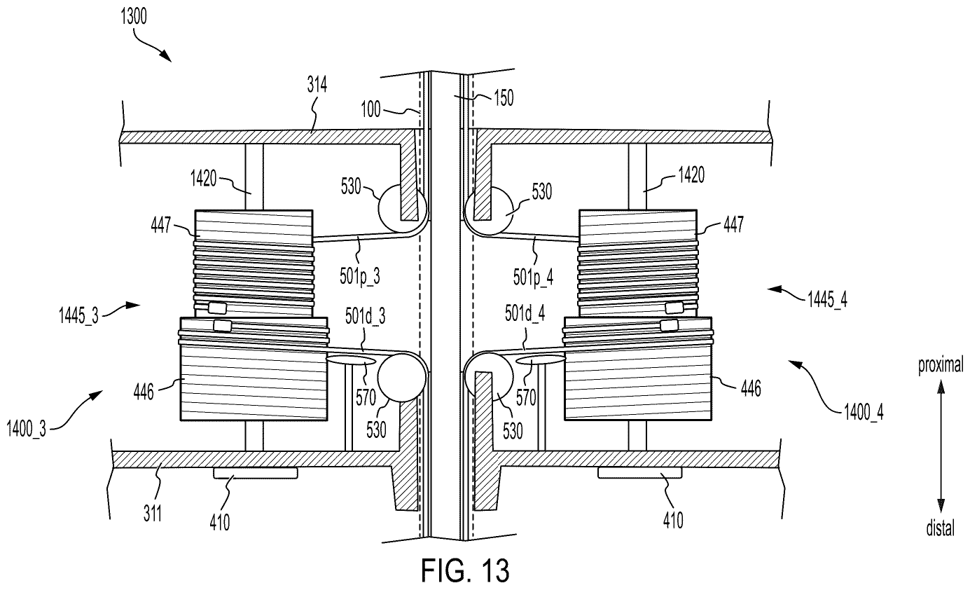

Although in FIG. 5, the proximally extending end portion 501p_3 and 501p_4 are coupled with the more distally positioned actuation element winding portions 446 of the capstan 445_3 and 445_4 and the distally extending end portions 501d_3 and 501d_4 are coupled with the more proximally positioned actuation element winding portions 447 of the capstans 445_3 and 445_4, in other embodiments this arrangement may be reversed. For example, in the embodiment illustrated in FIG. 13, the distally extending end portions 501d_3 and 501d_4 are coupled with the more distally positioned actuation element winding portions 446 of the capstans 1445_3 and 1445_4, while the proximally extending end portions 501p_3 and 501p_4 are coupled with the more proximally positioned actuation element winding portions 447 of the capstans 1445_3 and 1445_4. The embodiment of FIG. 13 is described in greater detail below.

Although in the embodiments of FIGS. 4, 5, 8A, and 8B capstans 445 are stepped capstans, in other embodiments described in greater detail below, the capstans may be non-stepped capstans.

### Pushable Actuation Element and Associated Pulleys

Turning now to FIGS. 9A-10D, an embodiment of the pushable actuation element 150 and an arrangement of the pulleys 535 and 536 relative to the pushable actuation element 150 are described. FIGS. 9A-9F illustrate different portions of the pushable actuation element 150, the actuation elements 501, the pulleys 535 and 536 that route the actuation elements 501, and other related aspects. FIGS. 10A-10D illustrate portions of the pushable actuation element 150.

The pushable actuation element 150 has a multi-part structure comprising one or more relatively rigid push-pull or push-push members coupled with and one or more laterally flexible push-push or push-pull members. For example, as shown in FIGS. 9A-9F, in some embodiments the actuation element 150 comprises proximal end portion 153, which comprises a relatively rigid member, such as a tube or rod. As shown in FIG. 9D, 9E and 10A-10D, the actuation element 150 also comprises a more distal portion 155, which may comprise a more flexible member, such as a beam that is flexible in one dimension, a push-coil, a cut tube, a pullable actuation element that is laterally supported to prevent buckling, or the like. As shown in FIG. 9D, 9E and 10A-10D, the pushable actuation element 150 may also comprise an intermediate portion 154, which is coupled to the proximal portion 153 and the distal portion 155. As shown in FIGS. 10A-10D, in some embodiments, the intermediate portion 154 comprises two rigid members 154a and 154b that extend in the proximal-distal direction on opposite sides of the distal pulley 535d, thereby allowing the actuation element 150 to move proximally and distally relative to the distal pulley 535d, as described in greater detail below.

As shown in FIGS. 9A-9C, the proximal pulley 536p may be coupled with the actuation element 150 at a proximal coupling position 151 of the proximal portion 153. In FIGS. 9A-9C, the proximal coupling position 151 is located at the end of the proximal portion 153, but in other embodiments the proximal coupling position 151 is not necessarily at the end of the actuation element 150. As shown in FIG. 9A, the actuation element 501p_3 is coupled with the proximal pulley 536p.

As shown in FIGS. 9A-9C, the proximal pulley 535p is coupled with the instrument shaft 100 at a proximal coupling position 102. For example, in some embodiments the proximal coupling position 102 may be part of an end cap 103 that is coupled to a proximal end of the shaft 100, as shown in FIG. 9B, in which case the proximal pulley 535p is coupled to the shaft 100 via the end cap 103.

FIGS. 9A and 9B illustrate the actuation element 150 in its most proximal position, and in this position, the proximal pulley 536p is located distal of and adjacent to the proximal pulley 535p. FIG. 9C illustrates the actuation element 150 in most distal position, and in this position, the proximal pulley 536p is spaced apart from the proximal pulley 535p by a distance D₁.

FIG. 9C also shows the locations of the drive members 400_1 to 400_4 and waterfall pulleys 530 in a most proximal position of the transmission assembly 300 relative to the instrument shaft 100. As shown in FIG. 9C, the proximally extending portions of the actuation elements 501 are initially positioned on an exterior of the shaft 100 as they leave the waterfall pulleys 530, and they extend proximally along the exterior of the shaft 100 until reaching openings 106 in the external wall of the shaft 100 through which the actuation elements 501 enter an interior of the shaft 100. As shown in FIG. 9C, in some embodiments, the openings 106 are positioned between the waterfall pulleys 530 and the pulley 536p when the actuation element 150 is in its most distal position and the transmission assembly 300 in its most proximal position. In other words, in most proximal position of the transmission assembly 300 and the most distal position of the actuation element 150, the waterfall pulleys 530 remain distal of the proximal pulley 536p. The above-described arrangement ensures that the actuation elements 501 remain able to actuate the actuation element 150 along its fully range of motion regardless of the position of the transmission assembly 300 along the shaft 100.

FIGS. 9D-9E illustrate a distal end portion of the actuation element 150, with FIG. 9D shown the actuation element 150 is in its most proximal position and FIG. 9E showing the actuation element 150 in its most distal position. As shown in FIGS. 9D and 9E, the distal pulley 536d is coupled to the actuation element 150 at a distal coupling location 152. The distal coupling location 152 is located where the proximal portion 153 meets the intermediate portion 154. The distal pulley 535d is coupled to the instrument shaft 100 at a distal coupling location 101. As shown in FIG. 2, the distal coupling location 101 may be located at a distal portion 100a of the instrument shaft 100. As shown in FIGS. 9D and 9E, the distal pulley 535d is coupled to the instrument shaft 100 by a coupling member 537. The coupling member 537 extends through the space between the two members 154a and 154b intermediate portion 154 of the actuation element 150 to couple with opposite sides of the instrument shaft 100. As shown in FIG. 9D, when the actuation element 150 is at its most proximal position, the distal pulley 536d is spaced apart from the distal pulley 535d by a distance D₃. In some embodiments, the distance D₃ may be approximately the same as the distance D₁ described above. As shown in FIG. 9E, when the actuation element 150 is at its most distal position, the distal pulley 536d is adjacent to the distal pulley 535d.

FIG. 9F illustrates the drive members 400, actuation elements 501, and waterfall pulleys 530 in isolation and in a state in which the force transmission assembly 300 is at a most distal position relative to the shaft and the actuation element 150 (omitted from FIG. 9F for clarity) is at its most proximal position. As shown in FIG. 9F, the distally extending portions of the actuation elements 501 are initially positioned on an exterior of the shaft 100 as they leave the waterfall pulleys 530, and they extend distally along the exterior of the shaft 100 until reaching openings 107 in the external wall of the shaft 100 through which the actuation elements 501 enter an interior of the shaft 100. As shown in FIG. 9F, in some embodiments, the openings 107 are positioned between the waterfall pulleys 530 and the distal pulley 536d when the actuation element 150 is in its most proximal position and the transmission assembly 300 in its most distal position. In other words, in the most distal position of the transmission assembly 300 and the most proximal position of the actuation element 150, the waterfall pulleys 530 remain proximal of the distal pulley 536d.

Turning now to FIGS. 10A-10D, the intermediate portion 154 and motion of the actuation element 150 will be described in greater detail. As mentioned above, in some embodiments the intermediate portion 154 of the actuation element 150 comprises two members 154a and 154b, which extend approximately in parallel with one another along opposite sides of the distal pulley 535d and the coupling member 537 that couples the distal pulley 535d to the instrument shaft 100, as shown in FIGS. 10A-10D. The members 154a and 154b define a space therebetween within which the distal pulley 535d and coupling member 537 are located, with this space allowing the actuation element 150 to move relative to the distal pulley 535d and coupling member 537 without interference. FIGS. 10A and 10C illustrate the actuation element 150 in a proximal position, and FIGS. 10B and 10D illustrate the actuation element 150 in a distal position reached as a result of translating the actuation element 150 distally from the state shown in FIGS. 10A and 10C.

The embodiment of FIG. 4-10D is described above as one non-limiting example of how the drive members 400 may be arranged to interact with the pullable actuation elements 500 and 501 and pushable actuation element 150, but other embodiments are contemplated herein. Specifically, although five drive members 400 are illustrated in FIGS. 4-10D, other embodiments have more or fewer drive members 400, including any number of drive members 400_1 to 400_N where N is any integer equal to or greater than 2. In addition, although FIGS. 4-10D illustrate specific numbers of actuation elements 500 and 501 in various arrangements (e.g., some extending distally, some proximally, etc.), in other embodiments more or fewer actuation elements 500 and/or 501 may be provided and different numbers of actuation elements 500 and/or 501 may extend in the proximal or distal directions than those illustrated. In addition, although FIG. 6 illustrates the actuation elements 500 as all being coupled directly to an articulable structure 230, in other embodiments one or more actuation elements 500 could be coupled to something other than such an articulable structure 230, such as another type of movable component 200 (e.g., a movable component of an end effector 210), another actuation element (e.g., push-pull rod), the instrument shaft 100, or another component of the instrument 10.

### End Effector and Articulable Structures

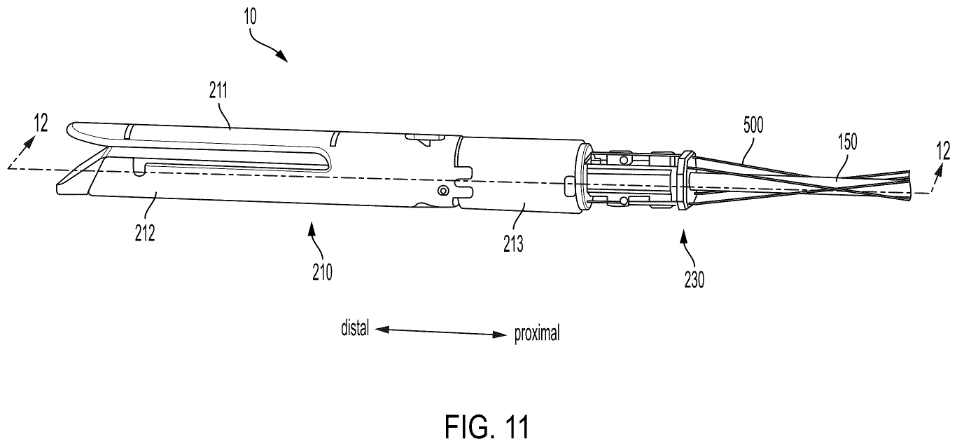

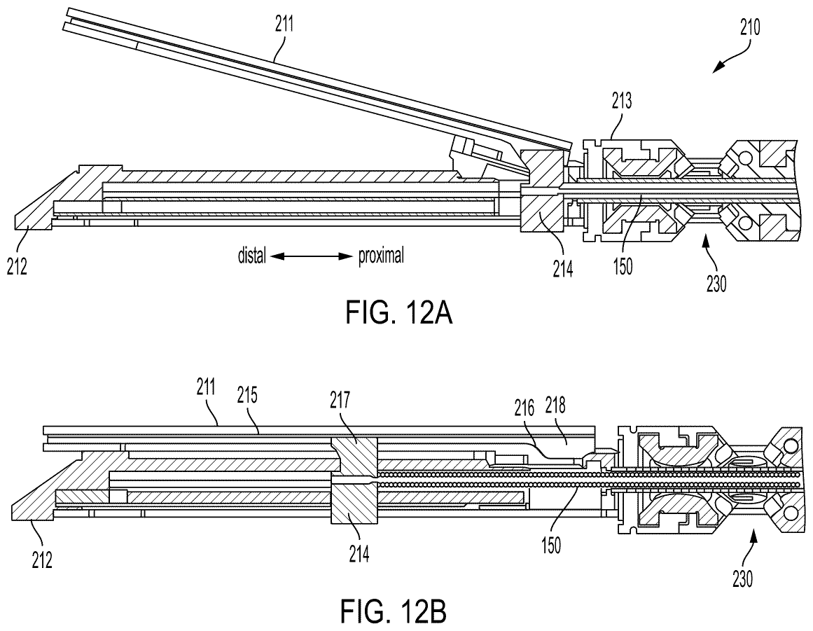

Turning now to FIG. 11-12B, embodiments of an end effector 210 and articulable structures 230 are described. The end effector 210 may be coupled to a distal end portion of the shaft 100, for example via the articulable structures 230.

In the embodiment of FIGS. 11-12B, the end effector 210 is configured as a stapler that grasps, staples, and cuts an object (e.g., tissue). The end effector 210 comprises a jaw mechanism with two opposing jaw members 211 and 212 that are pivotally coupled together so as to pivot between open and closed configurations. For example, in some embodiments the jaw member 211 is pivotably coupled to a clevis 213 and the jaw member 212 is stationary relative to the clevis 213. In the open configuration, distal ends of the jaw members 211 and 212 are spaced apart from one another, while in the closed configuration the distal ends of the jaw members 211 and 212 are relatively close to one another.

The jaw members 211 and 212 are driven to move between the open and closed configurations by translation of a translatable component 214. The translatable element 214 is a staple firing element to fire staples into material grasped between the jaw members 211 and 212. In the embodiment of FIGS. 11-12B, the translating element 214 also comprises a blade to cut material held between the jaw members 211 and 212 and into which staples have been (or are being) fired. The translatable element 214 may be driven to translate between a proximal or home position, illustrated in FIG. 12A, and a distal or extended position, illustrated in FIG. 12B. As shown in FIG. 12B, the jaw member 211 comprises a track 215 that engages with and guides a portion 217 of the translatable element 214 as the translatable element 214 translates relative to the jaw member 211. The track 215 comprises a ramp 216 and a garage area 218. When the translating element 214 is in its most proximal position, the portion 217 is located in the garage area 218. If the translating element 214 is pulled proximally while the portion 217 is located in the garage area 218, this pulls the jaw member 211 into the open position, as shown in FIG. 12A. When the translating element 214 is driven to translate distally from the garage area 218, the portion 217 engages with the ramp 216 such that the jaw member 211 is forced to pivot into the closed position. The jaw member 211 is thereafter held in the closed position as the translating element 214 translates along the jaw member 211 by interaction between the portion 217 and the track 215. As the translating element 214 translates distally, the translating element 214 fires staples (not illustrated) contained within the jaw member 212 (e.g., in a cartridge loaded into the jaw member 212, or in other receptacles in the jaw member 212) by colliding with the staples or with other components (e.g., staple raising components) in the jaw member 212 and thereby pushing the staples toward and into the material grasped between the jaw members 211 and 212. The jaw member 211 may serve as an anvil that bends the staples into a closed configuration.

The translatable element 214 is driven to move between the proximal and distal positions by translation of the actuation element 150. As shown in FIGS. 12A and 12B, the translatable element 214 is coupled to a distal end of the actuation element 150. As noted above, the pushable actuation element 150 can transfer both pushing forces and pulling forces, and thus is able to drive both proximal and distal translation of the translatable element 214 relative to the jaw members 211 and 212.

As shown in FIGS. 11-12B, in some embodiments, the articulable structures 230 comprises joints. For example, in some embodiments the articulable structures 230 comprises two orthogonal joints coupled together to form a wrist mechanism having two orthogonal degrees of freedom of motion (e.g., pitch and yaw). These joints and are coupled to and driven by actuation elements 500, such as the actuation elements 500_2, 500_2′, 500_5, and 500_5′ as described above.

The end effector 210 illustrated in FIGS. 11-12B is configured as a stapler, but this is just one example of a type of end effector that can be used in the instrument 10. One of ordinary skill in the art would understand that any other type of end effector could be used with the instrument 10 in lieu of the end effector 210, such as, for example, an end effector configured as a vessel sealer, an electrosurgical device (bipolar or monopolar), an electrocautery device, an endoscope, forceps, or any other type of end effector. Some examples of vessel sealers that may be used as the end effector 210 are described in U.S. Pat. No. 9,055,961 and U.S. Patent Application No. 63/279,500 titled Instrument End Effector With Jaw Mechanism and Movable Component and Related Devices, Systems, and Methods filed Nov. 15, 2021, the contents of both of which are incorporated herein by reference in their entirety. Similarly, any number of articulable structures of any type may be used in addition to or in lieu of the articulable structures 230, including having zero articulable structures in some embodiments.

### Additional Embodiments of Force Transmission Assemblies

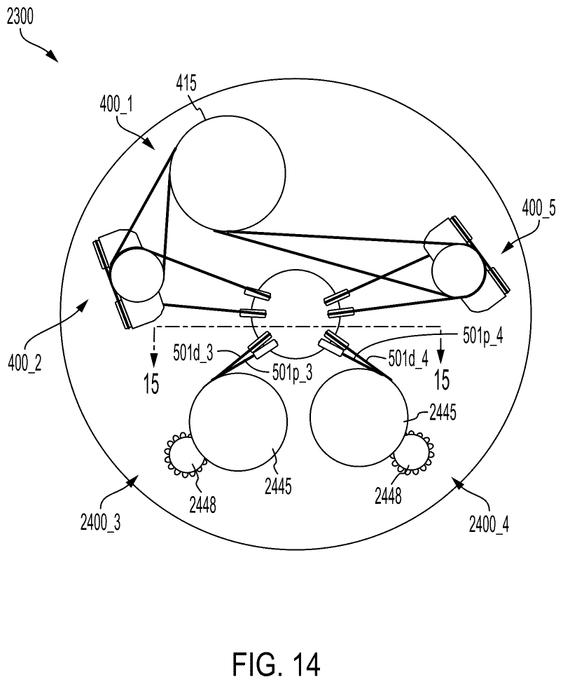

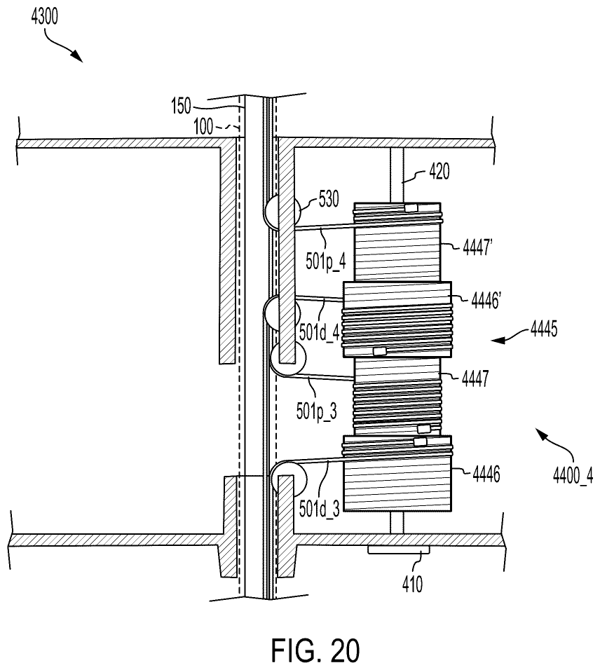

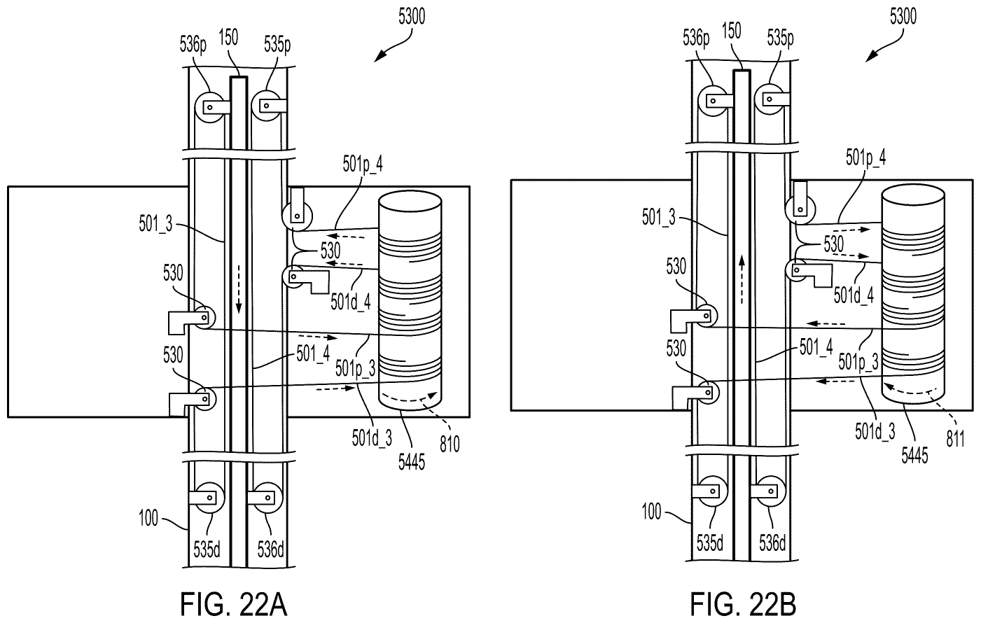

Turning now to FIGS. 13-23B, additional embodiments of force transmission assemblies are described, namely the force transmission assemblies 1300 (FIG. 13), 2300 (FIGS. 14-16B), 3300 (17-19B), 4400 (FIGS. 20-21B), and 5300 (FIGS. 22A-22B). The force transmission assemblies 1300, 2300, 3300, 4300, and 5300 are similar to the force transmission assembly 300 described above, and parts of the transmission assemblies 300, 1300, 2300, 3300, 4300, and 5300 that are the same as or similar to one another are given the same reference numerals and duplicative description thereof is omitted. Parts of the transmission assemblies 1300, 2300, 3300, 4300, and 5300 that are similar in form or function to corresponding parts of the transmission assembly 300, but which differ therefrom in some relevant way, may be given reference numbers that have the same three right-most digits as the corresponding parts of the transmission assembly 300. The force transmission assemblies 1300, 2300, 3300, 4300, and 5300 differ from the force transmission assembly 300 and from one another primarily in that they have different configurations of capstans to actuate the actuation elements 501 to drive translation of the actuation element 150. Any of the force transmission assemblies 300, 1300, 2300, 3300, 4300, and 5300 may be used as the force transmission assembly 23 described above.

Turning now to FIG. 13, the force transmission assembly 1300 will be described. The force transmission assembly 1300 is similar to the force transmission assembly 300 except that in the force transmission assembly 1300 the drive members 1400_3 and 1400_4 replace the drive members 400_3 and 400_4. The drive members 1400_3 and 1400_4 comprise capstans 1445, which are similar to the capstans 445 except that the capstans 1445 are so-called traveling capstans, which are configured to move longitudinally along their longitudinal axes as they are rotated to wind or unwind the actuation elements 501 coupled thereto so as to prevent a change in the angle at which the actuation elements 501 join or leave the capstans 1445. Thus, in this embodiment, the capstans 1445 are coupled to the drive shafts 1420 such that the capstans 1445 can translate along the drive shafts 1420 while still being constrained to rotate along with the drive shafts 1420. For example, the capstans 1445 and/or drive shafts 1402 may comprise a spline mechanism, such as a ball spline mechanism, recirculating ball spline mechanism, etc. A guide element 570 is coupled to the chassis 311 and engaged with a groove in the capstan 1445 along which the actuation elements 501 are wound, with the guide element 570 forcing the capstan 1445 to translate along the drive shaft 1420 in response to rotation of the capstan 1445. Other types of traveling capstans known in the art may also be used in lieu of the capstan 1445. Examples of traveling capstans that can be used as the capstans 1445 are described in U.S. Pat. No. 8,151,661 and U.S. Patent Application No. 63/157,858 titled Devises, Systems and Methods for Controlling Cable Drive Mechanisms filed Mar. 8, 2021, the contents of both of which are incorporated herein by reference in their entirety. The other components of the force transmission assembly 1300 are similar to those of the transmission assembly 300, and duplicative description thereof is omitted.

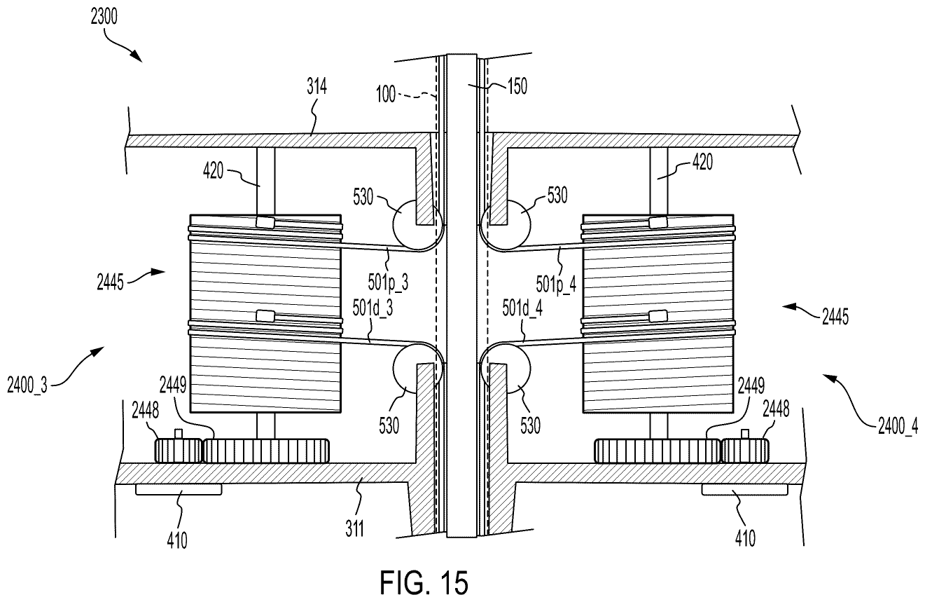

Turning now to FIGS. 14-16B, the force transmission assembly 2300 will be described. The force transmission assembly 2300 is similar to the force transmission assembly 300 except that in the force transmission assembly 2300 the drive members 2400_3 and 2400_4 replace the drive members 400_3 and 400_4, respectively. The drive members 2400_3 and 2400_4 comprise capstans 2445 that are not stepped, unlike the capstans 445. Instead, as shown in FIG. 15, the cable handling portions of a given capstan 2245 have the same radius as one another. As noted above, the stepped capstans 445 provide a mechanical advantage that increases the amount of force they are able to transmit. Thus, in some embodiments in which non-stepped capstans 2245 are used, a gear mechanism may be added to make up for the loss in mechanical advantage that results from using non-stepped capstans. For example, as shown in FIGS. 14 and 15, in some embodiments the drive members 2400_3 and 2400_4 comprise a first gear 2448 with a first radius and first tooth count coupled to the drive input 410, and a second gear 2449 with a second radius and a second tooth count coupled to the drive shaft 420 of the capstan 2445. The first and second gears 2448 and 2449 are meshed together, such that rotation of the drive input 410 drives rotation of the drive shaft 420. The first radius is less than the second radius and the first tooth count is less than the second tooth count, and therefore the first and second gears 2448 and 2449 provide a mechanical advantage. The mechanical advantage is proportional to the difference in the radius/tooth count between the gears 2448 and 2449.

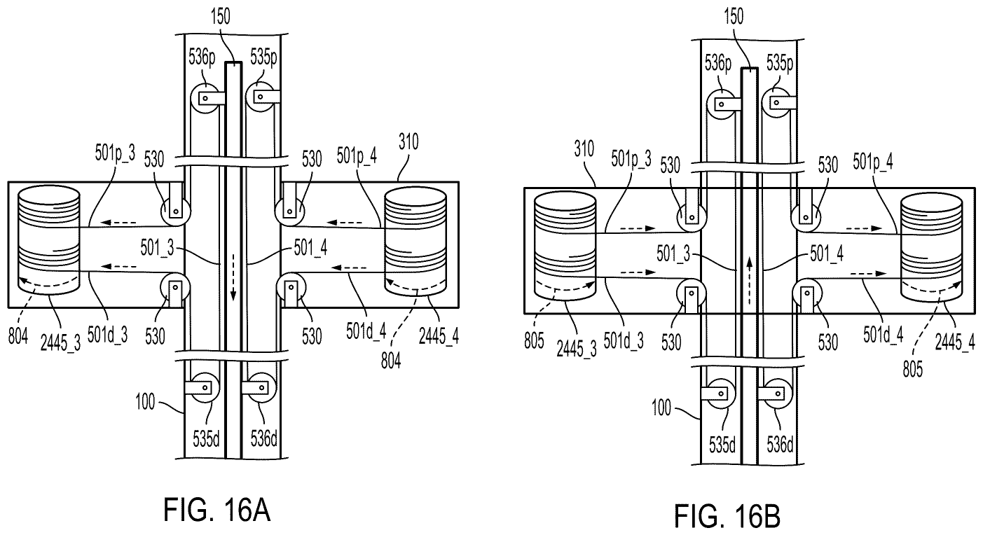

As shown in FIG. 15, because the capstans 2445 are not stepped, the end portions of the actuation element 501 may be coupled to the capstans 2445 differently than they are to the capstans 445. Specifically, for a given capstan 2445, two end portions of a corresponding actuation element 501 are wrapped around the capstan 2445 in the same direction as one another (instead of in opposite direction), so that the two end portions are simultaneously drawn in or simultaneously paid out in response to rotation of the capstan 2445. Thus, to translate the actuation element 150, both drive members 2400_3 and 2400_4 may be rotated together so as to pay out one of the actuation elements 501_3 and 501_4 while drawing in the other. In the embodiment of FIGS. 15 and 16, the actuation elements 501_3 and 501_4 are wrapped around the capstans 2445_3 and 2445_4 in opposite directions such that the capstans 2445_3 and 2445_4 can be simultaneously rotated together in the same direction as one another to actuate the actuation element 150. Specifically, as shown in FIG. 16A, when the two capstans 2445_3 and 2445_4 are actuated together in a first direction indicated by the arrow 804, the two end portions 501p_3 and 501d_3 are drawn in together by the capstan 2445_3, thus drawing in the overall length of the actuation element 501_3, while the two end portions 501p_4 and 501d_4 are paid out together by the capstan 2445_4, thus paying out the overall length of the actuation element 501_4. This creates tension in the actuation element 501_3 that urges the proximal pulley 536p, and hence the actuation element 150 coupled thereto, to translate distally relative to the shaft 100, and this distal motion is not inhibited by the actuation element 501_4 because it is simultaneously being paid out. Conversely, as shown in FIG. 16B, when the capstans 2445_3 and 2445_4 are rotated together in a second direction indicated by the arrow 805, they pay out the actuation element 501_3 while drawing in the actuation element 501_4, thus pulling the distal pulley 536d, and hence the actuation element 150 coupled thereto, to translate proximally relative to the shaft 100.

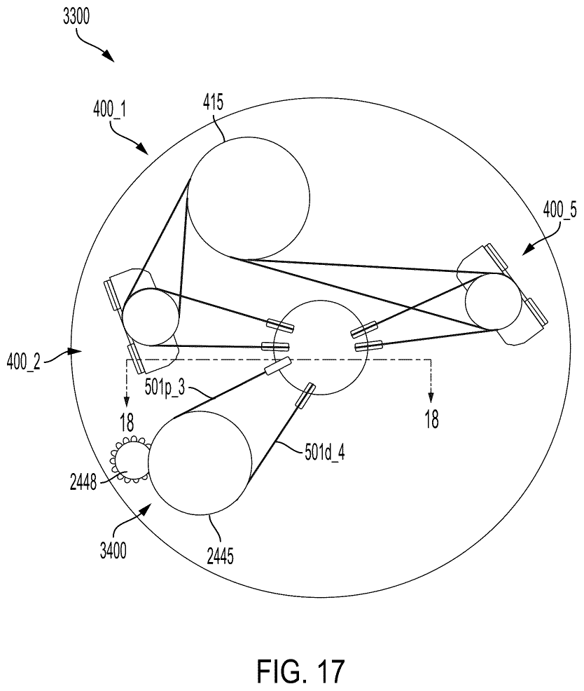

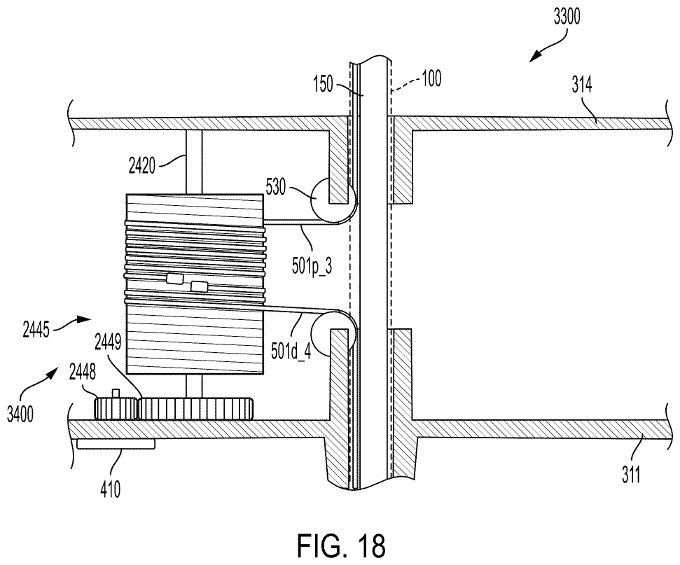

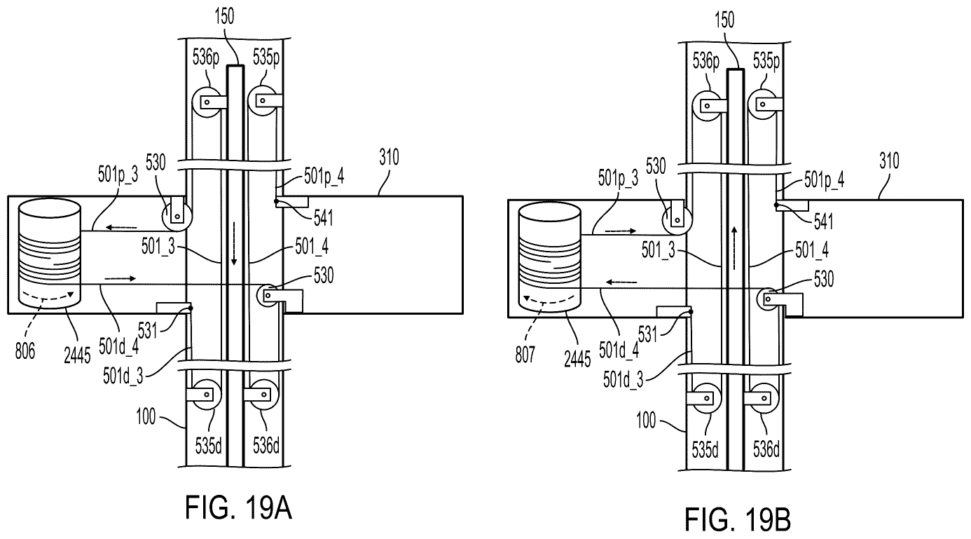

Turning now to FIGS. 17-19B, the force transmission assembly 3300 will be described. The force transmission assembly 2300 is similar to the force transmission assembly 2300 except that in the force transmission assembly 3300 the single drive member 3400 replaces both of the drive members 2400_3 and 2400_4. The drive member 3400 comprise a non-stepped capstan 2445, similar to the drive members 2400_3 and 2400_4. However, as shown in FIGS. 17-19B, instead of both ends of one actuation element 501_3 being coupled to one capstan 2445_3 and both ends the other actuation element 501_4 being coupled to another capstan 2445_4, in this embodiment a single end portion of each of the actuation elements 501_3 and 501_4 is coupled to the single capstan 2445, with the other ends of the actuation elements 501_3 and 501_4 being coupled to anchor points that are fixed relative to the transmission assembly 3300. Specifically, the end portions 501p_3 and 501d_4 are coupled to the capstan 2445, while the end portions 501d_3 and 501p_4 are coupled to anchor points 331 and 341, respectively. The end portions 501p_3 and 501d_4 are wrapped around the capstan 2445 in opposite directions, such that rotation of the capstan 2245 in a given direction actuates the end portions 501p_3 and 501d_4 in opposite directions (i.e., paying out one while drawing in the other). Thus, as shown in FIG. 19A, when the capstan 2445 is rotated in a first direction indicated by the arrow 806, the end portion 501p_3 is drawn in while the end portion 501d_4 is paid out, thus causing the actuation element 150 to translate distally. Conversely, as shown in FIG. 19B, when the capstan 2445 is rotated in a second direction indicated by the arrow 807, the end portion 501p_3 is paid out in while the end portion 501d_4 is drawn in, thus causing the actuation element 150 to translate proximally. In some embodiments, the drive member 3400 may also comprise a gear mechanism with a first gear 2448 and a second gear 2449, as shown in FIG. 18 and as described above with respect to the drive members 2400.

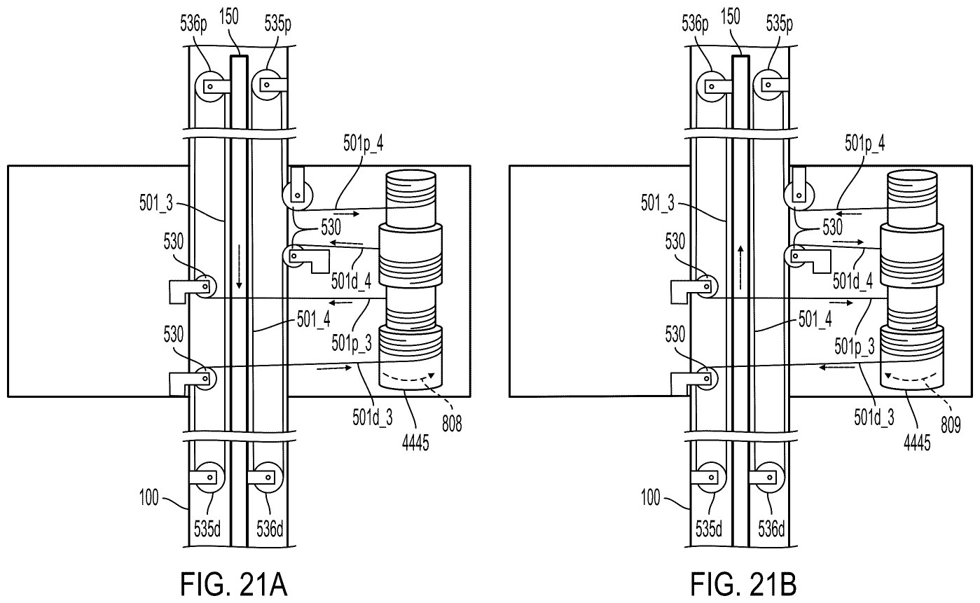

Turning now to FIGS. 20-21B, the force transmission assembly 4300 will be described. The force transmission assembly 4300 is similar to the force transmission assembly 300 except that in the force transmission assembly 4300 a single drive member 4400 replaces both of the drive members 400_3 and 400_4. The drive member 4400 comprises a stepped capstan 4445 that has all four end portions of the actuation elements 501_3 and 501_4 coupled thereto. Actuation element handling portions of the capstan 4445 include at least two portions having different radii, with the end portions of the actuation elements 501_3 and 501_4 coupled thereto being coupled to at least these to portions. For example, in FIG. 20, there are four actuation element handling portions 4466, 4446′, 4447, and 4447′, with the portions 4446 and 4446′ having a first radius and the portions 4446 and 4446′ having a second radius, smaller than the first radius. In some embodiments, rather than having four actuation element handling portions 4466, 4446′, 4447, and 4447′, the portions 4466 and 4446′ may be combined into a single portion and similar the portions 4447 and 4447′ may be combined into another portion. The end portions 501d_3 and 501p_4 are wrapped around the capstan 4445 in the same direction as one another, and similarly the end portions 501p_3 and 501d_4 are wrapped around the capstan 4445 in the same direction as one another. The end portions 501d_3 and 501p_4 are wrapped around the capstan 4445 in an opposite direction than the end portions 501p_3 and 501d_4. Thus, as shown in FIGS. 21A and 21B, when the capstan 4445 is rotated in a first direction indicated by the arrow 808, this causes the end portions 501d_3 and 501p_4 to be drawn in while the end portions 501p_3 and 501d_4 are paid out. Moreover, because of the differences in radii between the various actuation element handling portions 4446, 4446′, 4447, and 4447′, the end portions 501d_3 and 501p_4 are drawn in at a faster rate than the end portions 501p_3 and 501d_4 are paid out. Thus, the rotation capstan 4445 in the first direction indicated by the arrow 808 results in the actuation element 501_3 as a whole being drawn in (its overall paid out length decreases) and the actuation element 501_4 as a whole being paid out (its overall paid out length increases), and therefore the actuation element 150 is driven to translate distally as shown in FIG. 21B. Conversely, as shown in FIG. 21B, when the capstan 4445 is rotated in a second direction indicated by the arrow 809, the end portions 501d_3, 501p_3, 501d_4, and 501p_4 are actuated in the opposite manner as that described above, resulting in the actuation element 501_3 being paid out and the actuation element 501_4 being drawn in, thus driving the actuation element 150 to translate proximally.

Turning now to FIGS. 22A and 22B, the force transmission assembly 5300 will be described. The force transmission assembly 5300 is similar to the force transmission assembly 2300 except that in the force transmission assembly 5300 a single drive member with a single capstan 5445 replaces both of the drive members 2400_3 and 2400_4 and their respective capstans 2445. In this arrangement, all end portions of both of the actuation element 501_3 and 501_4 are coupled to the capstan 5445, with the end portions of the actuation element 501_3 being wrapped in a first direction and the end portions of the actuation element 501_4 being wrapped in a second direction opposite to the first direction. Thus, when the capstan 5445 is rotated in a first direction indicated by the arrow 810, the actuation element 501_3 is drawn in while the actuation element 501_4 is paid out, resulting in distal translation of the actuation element 150. Conversely, when the capstan 5445 is rotated in a second direction indicated by the arrow 811, the actuation element 501_3 is paid out while the actuation element 501_4 is drawn in, resulting in proximal translation of the actuation element 150. In some embodiments, gear mechanisms may be used in conjunction with the capstan 5445 to provide increased mechanical advantage, similar to the embodiment of FIGS. 17 and 18.

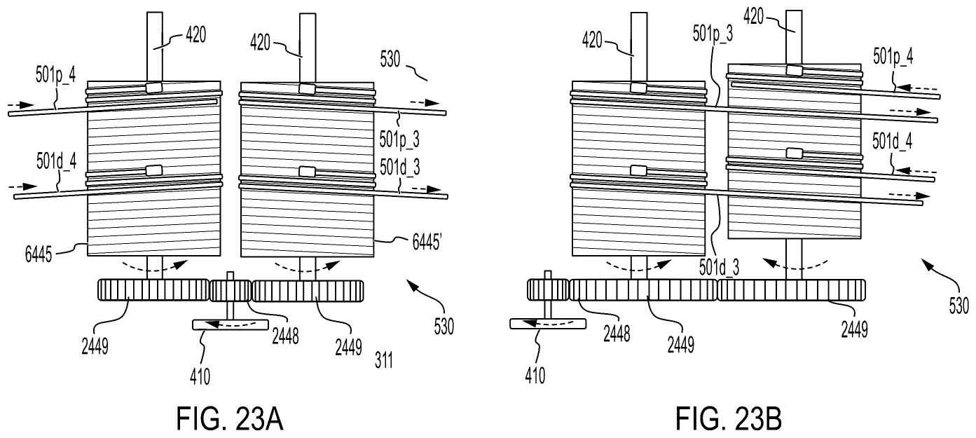

In various embodiments described above, multiple end portions of actuation elements 501 are described as being coupled to the same capstan (whether stepped or non-stepped). However, it should be understood that the same operational effect can be achieved by coupling the end portions to two separate capstans that are mechanically linked to one another such that they are driven to rotate together by the same drive input. For example, two discrete capstans coupled to the same drive shaft and drive input as one another will function as if they are a single capstan, and thus such an arrangement is regarded herein as the equivalent as a single capstan. As another example, as shown in the embodiment of FIGS. 23A and 23B, two discrete capstans 6445 may be coupled to the different drive shafts 420 but may be mechanically linked by gears 2448 and 2449 such that they are driven by same drive input 410. The two capstans 6445 may be coupled such that they rotate in the same direction as one another, as shown in FIG. 23A, or they may be coupled such that they rotate in opposite directions as one another, as shown in FIG. 23B. The directions that actuation elements 510 are wrapped around the capstans 6445 may vary based on whether the capstans 6445 rotate in the same direction or in difference directions, as shown in FIGS. 23A and 23B. Although FIGS. 23A and 23B illustrate non-stepped capstans that each have two end portions coupled thereto, the same principle applies to stepped capstans and/or to capstans having a single end portion coupled thereto. Moreover, although only two capstans 6445 are illustrated in FIGS. 23A and 23B as being coupled to the same drive input 410, any number of capstans could be coupled to the same drive input.

In some of the embodiments described above, stepped capstans have been described as having multiple actuation element handling portions with different radii. Although these stepped capstans are illustrated as a single unitary body with different portions, it should be understood that the stepped capstans could also be formed as an assembly of multiple individual capstans that are all mechanically linked to one another such that they are driven to rotate together by the same drive input. For example, the individual capstans could be coupled to the same drive shaft as another. As another example, the individual capstans could be coupled to different drive shafts that are mechanically linked to rotate together under the control of the same drive input. Such an assembly is regarded herein as equivalent to a single stepped capstan, with each discrete capstan making up the assembly serving as one of the steps (actuation element handling portions) of the overall stepped capstan assembly.

Furthermore, in some embodiments, stepped capstans described above may be replaced by multiple discrete capstans (e.g., one for each step of the stepped capstan), wherein the multiple capstans are not necessarily mechanically linked together so as to be driven by the same drive input as one another. Instead, in such embodiments the same operational effect that is achieved by the stepped capstans may be obtained by driving the multiple capstans in a coordinated fashion that mimics the behavior of single stepped capstan.

In the embodiments described above, specific examples of how the actuation elements 501 are coupled to and wrapped around the various capstans are described, but it should be understood that these are non-limiting examples and the actuation elements 501 could be routed and wrapped differently to achieve the same operational effect, as would be understood by one of ordinary skill in the art. As one example, FIG. 4 illustrates the end portion 501p_3 as being wrapped counterclockwise around the capstan 445_3 (from a perspective proximal thereof) and the end portion 501d_3 as being wrapped clockwise around the capstan 445_3, but in other embodiments the same operational effect may be achieved by wrapping the end portion 501p_3 clockwise and the end portion 501d_3 counterclockwise and reversing the rotational directions of the capstan 445_3. As another example, in FIG. 18 the end portions 501p_3 and 501d_4 are wrapped in opposite directions around the capstan 2445, but in other embodiments the same operational effect may be achieved by wrapping the end portions 501p_3 and 501d_4 in the same direction around two different capstans that are driven to rotate in opposite directions as one another. Similar alterations could be made to other embodiments as well, as would be understood by those of ordinary skill in the art.

In addition, although not shown FIGS. 14-23B, the embodiments illustrated in FIGS. 14-23B may also be provided with a traveling capstan that can translate along its drive shaft, similar to how the capstan 1445 of FIG. 13 can translate relative to the drive shaft 1420. For example, the guide element 570 of FIG. 13 could be used in any of the embodiments described above, and any of the capstans and/or drive shafts described above could be configured with a spline mechanism that allows the capstans to translate along the drive shaft while still being constrained to rotate with the drive shaft.

Moreover, although gear mechanisms are illustrated in FIGS. 15 and 18 in relation to the transmission assemblies 2300 and 3300s, it should be understood that in some embodiments of the transmission assemblies 300, 1300, 4300, and 5300, gear mechanisms may also be provided, which may be similar to those of the transmission assemblies 2300 and 3300. Furthermore, although the gear mechanism is illustrated in FIGS. 15 and 18 as two gears 2448 and 2449, it should be understood that any number gears, of any type, and in any arrangement may be used. Moreover, although FIGS. 15 and 18 show the transmission assemblies 2300 and 3300 as having a gear mechanism, in some embodiments of the transmission assemblies 2300 and 3300 the gear mechanism is omitted.

### Manipulator System

FIG. 24 is a schematic block diagram of an embodiment of a computer-assisted instrument control system 1000 for remote control of instruments in accordance with various embodiments, also referred to herein as a teleoperable instrument system. Such a system can be a medical system that employs robotic technology, as those having ordinary skill in the art are familiar with. The system 1000 comprises a manipulator assembly 1001, a control system 1006, and a user input and feedback system 1004. The system 1000 may also include an auxiliary system 1008 to provide various supporting functionality to the instruments or the overall system. These components of the system 1000 are described in greater detail blow.

The manipulator assembly 1001 comprises one or more manipulators 1014. FIG. 24 illustrates three manipulators 1014, but any number of manipulators 1014 may be included. While a manipulator may comprise a single mechanical link, in the embodiment of FIG. 24, each manipulator 1014 comprises a kinematic structure of two or more links 1015 coupled together by one or more joints 1016. The joints 1016 may impart various degrees of freedom of movement to the manipulator 1014, allowing the manipulator 1014 to be moved around a workspace 1009. For example, some joints 1016 may provide for rotation of links 1015 relative to one another, other joints 1016 may provide for translation of links 1015 relative to one another, and some may provide for both rotation and translation. Some or all of the joints 1016 may be powered joints, meaning a powered drive element may control movement of the joint 1016 through the supply of motive power. Such powered drive elements may comprise, for example, electric motors, pneumatic or hydraulic actuators, etc. Additional joints 1016 may be unpowered joints. FIG. 24 illustrates each manipulator 1014 as having two links 1015 and one joint 1016, but in practice a manipulator may include more links 1015 and more joints 1016, depending on the needs of the system 1000. The more links 1015 and joints 1016 are included, the greater the degrees of freedom of movement of the manipulator 1014.

Each manipulator 1014 may be configured to support and operate one or more instruments 1010. The instruments 1010 may include various types of instruments, including for example industrial instruments and medical instruments (e.g., surgical instruments, imaging instruments, diagnostic instruments, therapeutic instruments, etc.). For example, the instruments 1 and 10 described above may be used as any of the instruments 1010. A manipulator 1014 may comprise an instrument manipulator interface to which an instrument 1010 can be removably coupled. The instrument manipulator interface may be located, for example, at a distal end portion of the manipulator 1014. The instrument manipulator interface may include drive outputs to provide driving forces to drive inputs of the instrument 1010 to control operations of the instrument 1010, such as moving an end-effector of the instrument, opening/closing jaws, driving translating and/or rotating components, etc. The drive outputs may be driven by actuators (e.g., electrical motors, hydraulic actuators, pneumatic actuators, etc.) and may interface with and mechanically transfer driving forces to corresponding drive inputs of the instrument 1010 (directly, or via intermediate drive outputs, which may be part of a sterile instrument adaptor (ISA) (not illustrated)). The ISA may be placed between the instrument 1010 and the instrument interface to maintain sterile separation between the instrument 1010 and the manipulator 1014. The instrument interface may also comprise other interface components (not illustrated), such as electrical interfaces to provide and/or receive electrical signals to/from the instrument 1010. In some embodiments, the manipulator assembly can include flux delivery transmission capability as well, such as, for example, to supply electricity, fluid, vacuum pressure, light, electromagnetic radiation, etc. to the end effector. In other embodiments, such flux delivery transmission may be provided to an instrument through another auxiliary system, described further below.

An example embodiment of an instrument manipulating portion 60 of a manipulator 1014, is illustrated in FIG. 25. The instrument manipulating portion 60 is configured to support and operate an instrument 10 mounted thereon. As shown in FIG. 23, the instrument manipulating portion 60 comprises a base 77 and an instrument holder assembly 70. The base is coupled with a link 1015 of a manipulator 1014, for example by a joint 1016. The instrument holder assembly 70 comprises an outer housing 72 coupled to the base 77 and an instrument holder 71 coupled to the outer housing 72. The instrument holder 71 comprises an interface 73 and an inner portion (not illustrated) that is received within the outer housing 72. The instrument holder 71 may be rotatable relative to the outer housing 72, thus imparting a roll degree of freedom of motion to an instrument 10 mounted thereon.

As shown in FIG. 25, the instrument holder 71 comprises an interface 73 configured to receive and interface with the chassis 311 of the instrument 10 in a mounted state. The interface 73 comprises drive outputs 75 configured to interface with the drive inputs of the instrument, such as drive inputs 410 of the instrument 10, in a mounted state of the instrument to the manipulator. The drive outputs 75 may interface directly with the drive inputs 410, or the drive outputs 75 may interface indirectly with the drive inputs via intermediate drive outputs of an ISA (i.e., the drive outputs 75 engage corresponding intermediate drive outputs of the ISA, and the intermediate drive outputs of the ISA engage corresponding drive inputs 410). The drive outputs 75 may have a shape that is complementary to a shape of the drive inputs 410 and/or a shape of the intermediate drive outputs to allow for mating engagement such that rotation of the drive outputs 75 drives rotation drive inputs 410 and/or intermediate drive outputs. The inner portion of the instrument holder 71 may comprises actuators (not illustrated) coupled to the drive outputs 75 to supply torque to the drive outputs 75. Motive power, such as electricity or pressurized hydraulic or pneumatic fluid, may be provided to the actuators via power supply lines, which may be routed through the base 77 and into the housing 72.

The instrument holder assembly 70 of the embodiment of FIG. 25 comprises a passage 76 extending through the instrument interface 73 and the outer housing 72. In FIG. 25, the passage 76 comprises a generally cylindrical bore, but in other embodiments the passage 76 may have other shapes, such as a slot with a U-shaped cross-section that is open along a lateral side of the instrument holder assembly 70 in addition to being open at opposite axial ends of the instrument holder assembly 70. A portion of the shaft 100 and the sleeve 312 are received in the passage 76 in the mounted state. As described above, the sleeve 312 comprises alignment features 316a and 316b, and the inner surface of the passage 76 may comprise complementary alignment features (not illustrated) to engage with the alignment features 316a and 316b so as to progressively align the instrument 10 as the sleeve 312 is inserted farther into the passage 76.

As shown in FIG. 25, a cannula 74 may be coupled to (or may be an integral part of) the instrument manipulating portion 60. The cannula 74 may be configured for insertion into a patient through an incision or natural orifice so as to allow the shaft 100 of the instrument to be inserted and advanced therethrough. As noted above, a distal end portion of the sleeve 312 of an instrument 10 may extend partially into the cannula 74, and a seal may be provided between the cannula 74 and the exterior of the sleeve 312.

The system 1000 can also include a user input and feedback system 1004 operably coupled to the control system 1006. The user input and feedback system 1004 comprises one or more input devices to receive input control commands to control operations of the manipulator assembly 1001. Such input devices may include but are not limited to, for example, telepresence input devices, triggers, grip input devices, buttons, switches, pedals, joysticks, trackballs, data gloves, trigger-guns, gaze detection devices, voice recognition devices, body motion or presence sensors, touchscreen technology, or any other type of device for registering user input. In some cases, an input device may be provided with the same degrees of freedom as the associated instrument that they control, and as the input device is actuated, the instrument, through drive inputs from the manipulator assembly, is controlled to follow or mimic the movement of the input device, which may provide the user a sense of directly controlling the instrument. Telepresence input devices may provide the operator with telepresence, meaning the perception that the input devices are integral with the instrument. The user input and feedback system 1004 may also include feedback devices, such as a display device (not shown) to display images (e.g., images of the workspace 1009 as captured by one of the instruments 1010), haptic feedback devices, audio feedback devices, other graphical user interface forms of feedback, etc.

The control system 1006 may control operations of the system 1000. In particular, the control system 1006 may send control signals (e.g., electrical signals) to the manipulator assembly 1001 to control movement of the joints 1016 and to control operations of the instruments 1010 (e.g., through drive interfaces at the manipulators 1014). In some embodiments, the control system 1006 may also control some or all operations of the user input and feedback system 1004, the auxiliary system 1008, or other parts of the system 1000. The control system 1006 may include an electronic controller to control and/or assist a user in controlling operations of the manipulator assembly 1001. The electronic controller comprises processing circuitry configured with logic for performing the various operations. The logic of the processing circuitry may comprise dedicated hardware to perform various operations, software (machine readable and/or processor executable instructions) to perform various operations, or any combination thereof. In examples in which the logic comprises software, the processing circuitry may include a processor to execute the software instructions and a memory device that stores the software. The processor may comprise one or more processing devices capable of executing machine readable instructions, such as, for example, a processor, a processor core, a central processing unit (CPU), a controller, a microcontroller, a system-on-chip (SoC), a digital signal processor (DSP), a graphics processing unit (GPU), etc. In examples in which the processing circuitry includes dedicated hardware, in addition to or in lieu of the processor, the dedicated hardware may include any electronic device that is configured to perform specific operations, such as an Application Specific Integrated Circuit (ASIC), a Field Programmable Gate Array (FPGA), a Complex Programmable Logic Device (CPLD), discrete logic circuits, a hardware accelerator, a hardware encoder, etc. The processing circuitry may also include any combination of dedicated hardware and processor plus software.

As noted above, differing degrees of user control versus autonomous control may be utilized in the system 1000, and embodiments disclosed herein may encompass fully user-controlled systems, fully autonomously-controlled systems, and systems having any combination of user and autonomous control. For operations that are user-controlled, the control system 1006 generates control signals in response to receiving a corresponding user input command via the user input and feedback system 1004. For operations that are autonomously controlled, the control system 1006 may execute pre-programmed logic (e.g., a software program) and may determine and send control commands based on the programming (e.g., in response to a detected state or stimulus specified in the programming). In some systems, some operations may be user controlled and others autonomously controlled. Moreover, some operations may be partially user controlled and partially autonomously controlled-for example, a user input command may initiate performance of a sequence of events, and then the control system 1006 may perform various operations associated with that sequence without needing further user input.

While the control system 1006 is illustrated as a separate element in FIG. 23, those having ordinary skill in the art would understand that the control system 1006 may be a distributed system, or portions thereof may be, among one or more of the auxiliary system 1008, the user input and feedback system 1004, and the manipulator system 1001.

The auxiliary system 1008 may comprise various auxiliary devices that may be used in operation of the system 1000. For example, the auxiliary system 1008 may include power supply units, auxiliary function units (e.g., functions such as irrigation, evacuation, energy supply, illumination, sensors, imaging, etc.). As one example, in a system 1000 for use in a medical procedure context, the auxiliary system 1008 may comprise a display device for use by medical staff assisting a procedure, while the user operating the input devices may utilize a separate display device that is part of the user input and feedback system 1004. As another example, in a system 1000 for use in a medical context, the auxiliary system 1008 may comprise flux supply units that provide surgical flux (e.g., electrical power) to instruments 1010. An auxiliary system 1008 as used herein may thus encompass a variety of components and does not need to be provided as an integral unit.

The embodiments described herein (including the instrument 10 and system 1000 described above) may be well suited for use in medical applications. In particular, some embodiments are suitable for use in, for example, surgical, teleoperated surgical, diagnostic, therapeutic, and/or biopsy procedures. Such procedures could be performed, for example, on human patients, animal patients, human cadavers, animal cadavers, and portions or human or animal anatomy. Some embodiments may also be suitable for use in, for example, for non-surgical diagnosis, cosmetic procedures, imaging of human or animal anatomy, gathering data from human or animal anatomy, training medical or non-medical personnel, and procedures on tissue removed from human or animal anatomies (without return to the human or animal anatomy). Even if suitable for use in such medical procedures, the embodiments may also be used for benchtop procedures on non-living material and forms that are not part of a human or animal anatomy. Moreover, some embodiments are also suitable for use in non-medical applications, such as industrial robotic uses, and sensing, inspecting, and/or manipulating non-tissue work pieces. In non-limiting embodiments, the techniques, methods, and devices described herein may be used in, or may be part of, a computer-assisted medical system employing robotic technology such as the da Vinci® Surgical Systems commercialized by Intuitive Surgical, Inc., of Sunnyvale, California. Those skilled in the art will understand, however, that aspects disclosed herein may be embodied and implemented in various ways and systems, including manually operated instruments and computer-assisted, teleoperated systems, in both medical and non-medical applications. Reference to the daVinci® Surgical Systems are illustrative and not to be considered as limiting the scope of the disclosure herein.

It is to be understood that both the general description and the detailed description provide example embodiments that are explanatory in nature and are intended to provide an understanding of the present disclosure without limiting the scope of the present disclosure. Various mechanical, compositional, structural, electrical, and operational changes may be made without departing from the spirit and scope of this description and the claims. In some instances, well-known circuits, structures, and techniques have not been shown or described in detail in order not to obscure the embodiments. Like numbers in two or more figures represent the same or similar elements.

In the description above, reference is made to “paying out” or “drawing in” actuation elements. As noted above, paying out or drawing in an actuation element refers to increasing or decreasing the length of a segment of the actuation element that extends between a take-off point on the drive member to which the cable is coupled and a coupling point at which the actuation element is operably coupled to the structure whose motion the actuation element drives. The take-off point on a drive member refers to the point at which the actuation element ceases to contact the drive member (or begins to contact the drive member, depending on the point of view). The take-off point of the drive member may move as the drive member is operated-for example, as a capstan is rotated and a actuation element is wound onto or wound off from the capstan, the take-off point on the capstan may move axially along the capstan. As noted above, the coupling point is a point at which the actuation element is operably coupled to the structure that the actuation element is configured to drive, such as a point at which the actuation element is coupled to a movable component (if the actuation element is to drive motion of the movable component), the point at which the actuation element is coupled to a shaft (if the actuation element is to drive translation of the shaft), the point at which the cable is coupled to another actuation element (if the actuation element is to drive motion of the other actuation element), and so on. For example, with reference to FIG. 6 to explain principles of operation, the coupling points 565_2 to 565_5 are illustrated, which are fixed to the articulable structures 230. In other embodiments, the coupling locations may be different. A coupling location can constitute an end point of a given segment of the actuation element, which may be, but does not necessarily have to be, an end point of the entire actuation element. For example, in the embodiment of FIG. 5, the coupling locations 565_2 to 565_5 constitute end points of the entire actuation elements 500_2 to 500_5. In other embodiments, one or more coupling locations are not the end point of their respective actuation element—for example, the coupling location may be a location where a portion of the actuation element meets a pulley attached to the component driven by the actuation element. Moreover, the operable coupling of the actuation element to the structure it drives at the coupling location does not necessarily entail the actuation element being fixedly attached to the structure. Instead, the actuation element may be movably coupled to the structure it drives, for example via a pulley attached to the structure as previously mentioned.

In some circumstances, multiple portions of an actuation element may be coupled to the same drive member or to separate drive members. For example, one end portion of the actuation element may be coupled to a drive member, extend from the drive member to the structure driven by the actuation element, and then loop back such that an opposite end portion of the actuation element is coupled to a drive member (the same or different drive member). In such cases, there are two segments of the actuation element that extend between the drive member(s) and the coupling locations. In such circumstances, references to “paying out” or “drawing in” the actuation element should be understood as referring to paying in or drawing out one of these segments of the actuation element. The particular segment that is being referenced may be understood from the context if not explicitly identified.

Further, the terminology used herein to describe aspects of the invention, such as spatial and relational terms, is chosen to aid the reader in understanding example embodiments of the invention but is not intended to limit the invention. For example, spatially terms-such as “beneath”, “below”, “lower”, “above”, “upper”, “proximal”, “distal”, “up”, “down”, and the like-may be used herein to describe directions or one element's or feature's spatial relationship to another element or feature as illustrated in the figures. These spatial terms are used relative to the figures and are not limited to a particular reference frame in the real world. Thus, for example, the direction “up” in the figures does not necessarily have to correspond to an “up” in a world reference frame (e.g., away from the Earth's surface). Furthermore, if a different reference frame is considered than the one illustrated in the figures, then the spatial terms used herein may need to be interpreted differently in that different reference frame. For example, the direction referred to as “up” in relation to one of the figures may correspond to a direction that is called “down” in relation to a different reference frame that is rotated 180 degrees from the figure's reference frame. As another example, if a device is turned over 180 degrees in a world reference frame as compared to how it was illustrated in the figures, then an item described herein as being “above” or “over” a second item in relation to the Figures would be “below” or “beneath” the second item in relation to the world reference frame. Thus, the same spatial relationship or direction can be described using different spatial terms depending on which reference frame is being considered. Moreover, the poses of items illustrated in the figure are chosen for convenience of illustration and description, but in an implementation in practice the items may be posed differently.

In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components, unless specifically noted otherwise. Mathematical and geometric terms are not necessarily intended to be used in accordance with their strict definitions unless the context of the description indicates otherwise, because a person having ordinary skill in the art would understand that, for example, a substantially similar element that functions in a substantially similar way could easily fall within the scope of a descriptive term even though the term also has a strict definition.

Elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment.

Unless otherwise noted herein or implied by the context, when terms of approximation such as “substantially,” “approximately,” “about,” “around,” “roughly,” and the like, are used in conjunction with a stated numerical value, property, or relationship, such as an end-point of a range or geometric properties/relationships (e.g., parallel, perpendicular, straight, etc.), this should be understood as meaning that mathematical exactitude is not required for the value, property, or relationship, and that instead a range of variation is being referred to that includes but is not strictly limited to the stated value, property, or relationship. In particular, the range of variation around the stated value, property, or relationship includes at least any inconsequential variations from the value, property, or relationship, such as variations that are equivalents to the stated value, property, or relationship. The range of variation around the stated value, property, or relationship also includes at least those variations that are typical in the relevant art for the type of item in question due to manufacturing or other tolerances.

As used herein, “transverse” refers to a positional relationship of two items in which one item is oriented crosswise at an angle relative to the other item, such as being substantially or generally perpendicular to the other item. As used herein, “transverse” includes, but does not require, an exactly perpendicular relationship. For example, unless otherwise noted herein or implied by the context, “transverse” may include at least positional relationships in which one item is oriented at nonparallel angle to the other item, such as for example, an angle ranging from 45° to 135° relative to the other item.

Further modifications and alternative embodiments will be apparent to those of ordinary skill in the art in view of the disclosure herein. For example, the devices and methods may include additional components or steps that were omitted from the diagrams and description for clarity of operation. Accordingly, this description is to be construed as illustrative only and is for the purpose of teaching those skilled in the art the general manner of carrying out the present teachings. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

It is to be understood that the particular examples and embodiments set forth herein are non-limiting, and modifications to structure, dimensions, materials, and methodologies may be made without departing from the scope of the present teachings.

Other embodiments in accordance with the present disclosure will be apparent to those skilled in the art from consideration of the specification and practice of the invention disclosed herein. It is intended that the specification and examples be considered as exemplary only, with the following claims being entitled to their fullest breadth, including equivalents, under the applicable law.

1. A medical instrument comprising:
an instrument shaft;
a movable component coupled to the instrument shaft;
a pushable actuation element coupled to the moveable component and translatable relative to the instrument shaft, wherein the pushable actuation element is configured to drive a degree of freedom of motion of the movable component in response to translation of the pushable actuation element relative to the instrument shaft;
a first set of pullable actuation elements coupled to the instrument shaft;
a second set of pullable actuation elements coupled to the pushable actuation element; and
a transmission assembly movably coupled to the instrument shaft and comprising:
a first rotatable drive member configured to actuate the first set of pullable actuation elements to drive translation of the instrument shaft relative to the transmission assembly; and
one or more capstans configured to actuate the second set of pullable actuation elements to drive translation of the pushable actuation element relative to the instrument shaft.

2. The medical instrument of claim 1, wherein the pushable actuation element is configured to remain stationary relative to the instrument shaft while the instrument shaft is driven to translate relative to the transmission assembly.

3. The medical instrument of claim 1, further comprising:
a first pair of pulleys coupled to the pushable actuation element; and
a second pair of pulleys coupled to the instrument shaft;
wherein the second set of pullable actuation elements are routed through the first and second pairs of pulleys.

4. The medical instrument of claim 3, wherein the second set of pullable actuation elements are actuatable to cause movement of the first pair of pulleys relative to the second pair of pulleys to drive translation of the pushable actuation element relative to the instrument shaft.

5. The medical instrument of claim 3, wherein:
the second set of pullable actuation elements comprises a first pullable actuation element and a second pullable actuation element; and
simultaneous actuation of the first and second pullable actuation elements in opposite directions drives translation of the pushable actuation element.

6. The medical instrument of claim 5, wherein the one or more capstans comprise:
a first capstan coupled to a first drive input and the first pullable actuation element; and
a second capstan coupled to a second drive input and the second pullable actuation element.

7. The medical instrument of claim 6, wherein:
a first end portion and a second end portion of the first pullable actuation element are coupled to the first capstan; and
a third end portion and a fourth end portion of the second pullable actuation element are coupled to the second capstan.

8. The medical instrument of claim 6, wherein the first and second capstans each comprise stepped capstans.

9. The medical instrument of claim 8, wherein:
a first end portion and a second end portion of the first pullable actuation element are respectively coupled to steps of the first capstan having mutually different radii and are wrapped in opposite directions as one another; and
a third end portion and a fourth end portion of the second pullable actuation element are respectively coupled to steps of the second capstan having mutually different radii and are wrapped in opposite directions as one another.

10. The medical instrument of claim 6, wherein the first and second capstans each comprise non-stepped capstans.

11. The medical instrument of claim 10, wherein:
a first end portion and a second end portion of the first pullable actuation element are coupled to the first capstan and wrapped in a same direction as one another; and
a third end portion and a fourth end portion of the second pullable actuation element are coupled to the second capstan and wrapped in a same direction as one another.

12. (canceled)

13. The medical instrument of claim 6, wherein:
the first capstan is coupled to the first drive input via a first drive shaft and configured to translate along the first drive shaft in response to rotation of the first capstan; and
the second capstan is coupled to the second drive input via second drive shaft and configured to translate along the second drive shaft in response to rotation of the second capstan.

14. The medical instrument of claim 1, wherein the one or more capstans comprises a first capstan coupled to a first drive input and coupled to the second set of pullable actuation elements.

15. The medical instrument of claim 14, wherein:
the second set of pullable actuation elements comprises a first pullable actuation element and a second pullable actuation element;
a first end portion of the first pullable actuation element is coupled to the first capstan and a second end portion of the first pullable actuation element is coupled to an anchor point on the transmission assembly; and
a third end portion of the second pullable actuation element is coupled to the first capstan and a fourth end portion of the second pullable actuation element is coupled to an anchor point on the transmission assembly.

16. The medical instrument of claim 15, wherein:
wherein the first end portion of the first pullable actuation element and the second end portion of the second pullable actuation element are wrapped around the first capstan in opposite directions as one another.

17. (canceled)

18. The medical instrument of claim 14, wherein:
the first capstan comprises a stepped capstan;
the second set of pullable actuation elements comprises a first pullable actuation element and a second pullable actuation element;
a first end portion and a second end portion of the first pullable actuation element are respectively coupled to steps of the first capstan having mutually different radii and are wrapped in opposite directions as one another; and
a third end portion and a fourth end portion of the second pullable actuation element are respectively coupled to steps of the first capstan having mutually different radii and are wrapped in opposite directions as one another.

19.-20. (canceled)

21. The medical instrument claim 3, wherein:
the second set of pullable actuation elements comprises a first pullable actuation element and a second pullable actuation element;
the first pullable actuation element is coupled to
a proximal pulley of the first pair of pulleys, which is coupled to a proximal portion of the pushable actuation element, and
a distal pulley of the second pair of pulleys, which is coupled to a distal portion of the instrument shaft; and
the second pullable actuation element is coupled to
a distal pulley of the first pair of pulleys, which is coupled to a distal portion of the pushable actuation element, and
a proximal pulley of the second pair of pulleys, which is coupled to a proximal portion of the instrument shaft.

22.-41. (canceled)

42. A method of operating a medical instrument comprising an instrument shaft, a force transmission assembly coupled to a proximal end portion of the instrument shaft and an end effector coupled to a distal end portion of the instrument shaft, the method comprising:
translating the instrument shaft relative of the force transmission assembly; and
translating a pushable actuation element relative to the instrument shaft to drive a degree of freedom of motion of a movable component of the end effector;
wherein, while translating the instrument shaft relative to the force transmission assembly, the pushable actuation element remains stationary relative to the instrument shaft.

43. The method of claim 42, wherein translating the instrument shaft relative of the force transmission assembly comprises rotating an insertion drive member of the force transmission assembly to actuate a first set of pullable actuation elements coupled to the instrument shaft.

44. The method of claim 43, wherein translating the pushable actuation element relative to the instrument shaft comprises rotating one or more additional drive members of the force transmission assembly to actuate first and second pullable actuation elements coupled to the pushable actuation element.

45. (canceled)
